SEQUENCE LISTING (463 sequences)

SEQ ID NO: 1 - protein, 1059 aa, synthetic construct
MEANPAGSGAGGGGSSGIGGEDGVHFQSYPFDFLEFLNHQRFEPMELYGEHAKAVAALPCAPGPPPQPPPQPPPPQYDYPPQSTFKPKAEVPSSSSSSSSSSSSSSSSSSSSSSSSSQAKKPDPPLPPAFGAPPPPLFDAAFPTPQWGIVDLSGHQHLFGNLKRGGPASGPGVTPGLGAPAGAPGPLPAPSQTPPGPPAAAACDPTKDDKGYFRRLKYLMERRFPCGVCQKSFKQSSHLVQHMLVHSGERPYECGVCGRTYNHVSSLIRHRRCHKDVPPAAGGPPQPGPHLPPLGLPAPAASAATAAAPSTVSSGPPATPVAPAPSADGSAAPAGVGVPPPATGGGDGPFACPLCWKVFKKPSHLHQHQIIHTGEKPFSCSVCSKSFNRRESLKRHVKTHSADLLRLPCGICGKAFRDASYLLKHQAAHAGAGAGGPRPVYPCDLCGKSYSAPQSLLRHKAAHAPPAAAAEAPKDGAASAPQPPPTFPPGPYLLPPDPPTTDSEKAAAAAAAVVYGAVPVPLLGAHPLLLGGAGTSGAGGSGASVPGKTFCCGICGRGFGRRETLKRHERIHTGEKPHQCPVCGKRFRESFHLSKHHVVHTRERPYKCELCGKVFGYPQSLTRHRQVHRLQLPCALAGAAGLPSTQGTPGACGPGASGTSAGPTDGLSYACSDCGEHFPDLFHVMSHKEVHMAEKPYGCDACGKTFGFIENLMWHKLVHQAAPERLLPPAPGGLQPPDGSSGTDAASVLDNGLAGEVGAAVAALAGVSGGEDAGGAAVAGAGGGASSGPERFSCATCGQSFKHFLGLVTHKYVHLVRRTLGCGLCGQSFAGAYDLLLHRRSHRQKRGFRCPVCGKRFWEAALLMRHQRCHTEQRPYRCGVCGRGFLRSWYLRQHRVVHTGERAFKCGVCAKRFAQSSSLAEHRRLHAVARPQRCSACGKTFRYRSNLLEHQRLHLGERAYRCEHCGKGFFYLSSVLRHQRAHEPPRPELRCPACLKAFKDPGYFRKHLAAHQGGRPFRCSSCGEGFANTYGLKKHRLAHKAENLGGPGAGAGTLAGKDA

SEQ ID NO: 2 - protein, 769 aa, synthetic construct
MEANPAGSGAGGGGSSGIGGEDGVHFQSYPFDFLEFLNHQRFEPMELYGEHAKAVAALPCAPGPPPQPPPQPPPPQYDYPPQSTFKPKAEVPSSSSSSSSSSSSSSSSSSSSSSSSSQAKKPDPPLPPAFGAPPPPLFDAAFPTPQWGIVDLSGHQHLFGNLKRGGPASGPGVTPGLGAPAGAPGPLPAPSQTPPGPPAAAACDPTKDDKGYFRRLKYLMERRFPCGVCQKSFKQSSHLVQHMLVHSGERPYECGVCGRTYNHVSSLIRHRRCHKDVPPAAGGPPQPGPHLPPLGLPAPAASAATAAAPSTVSSGPPATPVAPAPSADGSAAPAGVGVPPPATGGGDGPFACPLCWKVFKKPSHLHQHQIIHTGEKPFSCSVCSKSFNRRESLKRHVKTHSADLLRLPCGICGKAFRDASYLLKHQAAHAGAGAGGPRPVYPCDLCGKSYSAPQSLLRHKAAHAPPAAAAEAPKDGAASAPQPPPTFPPGPYLLPPDPPTTDSEKAAAAAAAVVYGAVPVPLLGAHPLLLGGAGTSGAGGSGASVPGKTFCCGICGRGFGRRETLKRHERIHTGEKPHQCPVCGKRFRESFHLSKHHVVHTRERPYKCELCGKVFGYPQSLTRHRQVHRLQLPCALAGAAGLPSTQGTPGACGPGASGTSAGPTDGLSYACSDCGEHFPDLFHVMSHKEVHMAEKPYGCDACGKTFGFIENLMWHKLVHQAAPERLLPPAPGGLQPPDGSSGTDAASVLDNGLAGEVGAAVAALAGVSG

SEQ ID NO: 3 - protein, 290 aa, synthetic construct
GEDAGGAAVAGAGGGASSGPERFSCATCGQSFKHFLGLVTHKYVHLVRRTLGCGLCGQSFAGAYDLLLHRRSHRQKRGFRCPVCGKRFWEAALLMRHQRCHTEQRPYRCGVCGRGFLRSWYLRQHRVVHTGERAFKCGVCAKRFAQSSSLAEHRRLHAVARPQRCSACGKTFRYRSNLLEHQRLHLGERAYRCEHCGKGFFYLSSVLRHQRAHEPPRPELRCPACLKAFKDPGYFRKHLAAHQGGRPFRCSSCGEGFANTYGLKKHRLAHKAENLGGPGAGAGTLAGKDA

SEQ ID NO: 4 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 5 - protein, 12 aa, synthetic construct
SGGGSGGSGSGS

SEQ ID NO: 6 - protein, 7 aa, synthetic construct
PKKKRKV

SEQ ID NO: 7 - protein, 20 aa, synthetic construct
AVKRPAATKKAGQAKKKKLD

SEQ ID NO: 8 - protein, 25 aa, synthetic construct
MSRRRKANPTKLSENAKKLAKEVEN

SEQ ID NO: 9 - protein, 9 aa, synthetic construct
PAAKRVKLD

SEQ ID NO: 10 - protein, 9 aa, synthetic construct
KLKIKRPVK

SEQ ID NO: 11 - protein, 16 aa, synthetic construct
KRPAATKKAGQAKKKK

SEQ ID NO: 12 - protein, 2470 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKSGGGSGGSGSGSMEANPAGSGAGGGGSSGIGGEDGVHFQSYPFDFLEFLNHQRFEPMELYGEHAKAVAALPCAPGPPPQPPPQPPPPQYDYPPQSTFKPKAEVPSSSSSSSSSSSSSSSSSSSSSSSSSQAKKPDPPLPPAFGAPPPPLFDAAFPTPQWGIVDLSGHQHLFGNLKRGGPASGPGVTPGLGAPAGAPGPLPAPSQTPPGPPAAAACDPTKDDKGYFRRLKYLMERRFPCGVCQKSFKQSSHLVQHMLVHSGERPYECGVCGRTYNHVSSLIRHRRCHKDVPPAAGGPPQPGPHLPPLGLPAPAASAATAAAPSTVSSGPPATPVAPAPSADGSAAPAGVGVPPPATGGGDGPFACPLCWKVFKKPSHLHQHQIIHTGEKPFSCSVCSKSFNRRESLKRHVKTHSADLLRLPCGICGKAFRDASYLLKHQAAHAGAGAGGPRPVYPCDLCGKSYSAPQSLLRHKAAHAPPAAAAEAPKDGAASAPQPPPTFPPGPYLLPPDPPTTDSEKAAAAAAAVVYGAVPVPLLGAHPLLLGGAGTSGAGGSGASVPGKTFCCGICGRGFGRRETLKRHERIHTGEKPHQCPVCGKRFRESFHLSKHHVVHTRERPYKCELCGKVFGYPQSLTRHRQVHRLQLPCALAGAAGLPSTQGTPGACGPGASGTSAGPTDGLSYACSDCGEHFPDLFHVMSHKEVHMAEKPYGCDACGKTFGFIENLMWHKLVHQAAPERLLPPAPGGLQPPDGSSGTDAASVLDNGLAGEVGAAVAALAGVSGGEDAGGAAVAGAGGGASSGPERFSCATCGQSFKHFLGLVTHKYVHLVRRTLGCGLCGQSFAGAYDLLLHRRSHRQKRGFRCPVCGKRFWEAALLMRHQRCHTEQRPYRCGVCGRGFLRSWYLRQHRVVHTGERAFKCGVCAKRFAQSSSLAEHRRLHAVARPQRCSACGKTFRYRSNLLEHQRLHLGERAYRCEHCGKGFFYLSSVLRHQRAHEPPRPELRCPACLKAFKDPGYFRKHLAAHQGGRPFRCSSCGEGFANTYGLKKHRLAHKAENLGGPGAGAGTLAGKDAPKKKRKVDYKDDDDK

SEQ ID NO: 13 - protein, 2180 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKSGGGSGGSGSGSMEANPAGSGAGGGGSSGIGGEDGVHFQSYPFDFLEFLNHQRFEPMELYGEHAKAVAALPCAPGPPPQPPPQPPPPQYDYPPQSTFKPKAEVPSSSSSSSSSSSSSSSSSSSSSSSSSQAKKPDPPLPPAFGAPPPPLFDAAFPTPQWGIVDLSGHQHLFGNLKRGGPASGPGVTPGLGAPAGAPGPLPAPSQTPPGPPAAAACDPTKDDKGYFRRLKYLMERRFPCGVCQKSFKQSSHLVQHMLVHSGERPYECGVCGRTYNHVSSLIRHRRCHKDVPPAAGGPPQPGPHLPPLGLPAPAASAATAAAPSTVSSGPPATPVAPAPSADGSAAPAGVGVPPPATGGGDGPFACPLCWKVFKKPSHLHQHQIIHTGEKPFSCSVCSKSFNRRESLKRHVKTHSADLLRLPCGICGKAFRDASYLLKHQAAHAGAGAGGPRPVYPCDLCGKSYSAPQSLLRHKAAHAPPAAAAEAPKDGAASAPQPPPTFPPGPYLLPPDPPTTDSEKAAAAAAAVVYGAVPVPLLGAHPLLLGGAGTSGAGGSGASVPGKTFCCGICGRGFGRRETLKRHERIHTGEKPHQCPVCGKRFRESFHLSKHHVVHTRERPYKCELCGKVFGYPQSLTRHRQVHRLQLPCALAGAAGLPSTQGTPGACGPGASGTSAGPTDGLSYACSDCGEHFPDLFHVMSHKEVHMAEKPYGCDACGKTFGFIENLMWHKLVHQAAPERLLPPAPGGLQPPDGSSGTDAASVLDNGLAGEVGAAVAALAGVSGPKKKRKVDYKDDDDK

SEQ ID NO: 14 - protein, 1701 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKSGGGSGGSGSGSGEDAGGAAVAGAGGGASSGPERFSCATCGQSFKHFLGLVTHKYVHLVRRTLGCGLCGQSFAGAYDLLLHRRSHRQKRGFRCPVCGKRFWEAALLMRHQRCHTEQRPYRCGVCGRGFLRSWYLRQHRVVHTGERAFKCGVCAKRFAQSSSLAEHRRLHAVARPQRCSACGKTFRYRSNLLEHQRLHLGERAYRCEHCGKGFFYLSSVLRHQRAHEPPRPELRCPACLKAFKDPGYFRKHLAAHQGGRPFRCSSCGEGFANTYGLKKHRLAHKAENLGGPGAGAGTLAGKDAPKKKRKVDYKDDDDK

SEQ ID NO: 15 - DNA, 20 bp, synthetic construct
  source 1..20: mol_type = other DNA
gtcaggaccccagaaaagat

SEQ ID NO: 16 - DNA, 20 bp, synthetic construct
  source 1..20: mol_type = other DNA
gcgcacaaggatggatgagt

SEQ ID NO: 17 - DNA, 20 bp, synthetic construct
  source 1..20: mol_type = other DNA
ggggactggaagcctaaatc

SEQ ID NO: 18 - DNA, 20 bp, synthetic construct
  source 1..20: mol_type = other DNA
gggggtgatccgcacaagga

SEQ ID NO: 19 - DNA, 20 bp, synthetic construct
  source 1..20: mol_type = other DNA
gtctgcctgtccaccccaaa

SEQ ID NO: 20 - protein, 20 aa, synthetic construct
CCGCACAAGGATGGATGAGT

SEQ ID NO: 21 - DNA, 20 bp, synthetic construct
  source 1..20: mol_type = other DNA
gacgcccagagcgtgtcgcg

SEQ ID NO: 22 - DNA, 20 bp, synthetic construct
  source 1..20: mol_type = other DNA
gaggcgggcattcaaagcgc

SEQ ID NO: 23 - DNA, 20 bp, synthetic construct
  source 1..20: mol_type = other DNA
tcgcccaccggaatcggccc

SEQ ID NO: 24 - DNA, 20 bp, synthetic construct
  source 1..20: mol_type = other DNA
atcctccacgccggcgcctc

SEQ ID NO: 25 - DNA, 20 bp, synthetic construct
  source 1..20: mol_type = other DNA
acttccgcttccgggcgggc

SEQ ID NO: 26 - DNA, 20 bp, synthetic construct
  source 1..20: mol_type = other DNA
gcacttccggtcgggccctc

SEQ ID NO: 27 - DNA, 23 bp, synthetic construct
  source 1..23: mol_type = other DNA
gacgcccagagcgtgtcgcgggg

SEQ ID NO: 28 - DNA, 23 bp, synthetic construct
  source 1..23: mol_type = other DNA
tcgcggggttcccgtagaagagg

SEQ ID NO: 29 - DNA, 23 bp, synthetic construct
  source 1..23: mol_type = other DNA
gaggcgggcattcaaagcgcagg

SEQ ID NO: 30 - DNA, 23 bp, synthetic construct
  source 1..23: mol_type = other DNA
tcgcccaccggaatcggcccggg

SEQ ID NO: 31 - DNA, 23 bp, synthetic construct
  source 1..23: mol_type = other DNA
ttcgcccaccggaatcggcccgg

SEQ ID NO: 32 - DNA, 23 bp, synthetic construct
  source 1..23: mol_type = other DNA
atgggggcgcacgactgctatgg

SEQ ID NO: 33 - DNA, 23 bp, synthetic construct
  source 1..23: mol_type = other DNA
cgcgacacgctctgggcgtccgg

SEQ ID NO: 34 - DNA, 23 bp, synthetic construct
  source 1..23: mol_type = other DNA
agccggaggtgtggcttaaccgg

SEQ ID NO: 35 - DNA, 23 bp, synthetic construct
  source 1..23: mol_type = other DNA
cgactgctatggaggcgatgggg

SEQ ID NO: 36 - DNA, 23 bp, synthetic construct
  source 1..23: mol_type = other DNA
aagcccgggccgattccggtggg

SEQ ID NO: 37 - DNA, 23 bp, synthetic construct
  source 1..23: mol_type = other DNA
ggaagtcgcgcccgttcccgagg

SEQ ID NO: 38 - DNA, 23 bp, synthetic construct
  source 1..23: mol_type = other DNA
acgactgctatggaggcgatggg

SEQ ID NO: 39 - DNA, 23 bp, synthetic construct
  source 1..23: mol_type = other DNA
acttccgcttccgggcgggcggg

SEQ ID NO: 40 - DNA, 23 bp, synthetic construct
  source 1..23: mol_type = other DNA
gttaagccacacctccggctcgg

SEQ ID NO: 41 - DNA, 23 bp, synthetic construct
  source 1..23: mol_type = other DNA
ctcagttcgcccaccggaatcgg

SEQ ID NO: 42 - DNA, 23 bp, synthetic construct
  source 1..23: mol_type = other DNA
ggaaccccgcgacacgctctggg

SEQ ID NO: 43 - DNA, 23 bp, synthetic construct
  source 1..23: mol_type = other DNA
ggggcgcacgactgctatggagg

SEQ ID NO: 44 - DNA, 23 bp, synthetic construct
  source 1..23: mol_type = other DNA
cggacgcccagagcgtgtcgcgg

SEQ ID NO: 45 - DNA, 23 bp, synthetic construct
  source 1..23: mol_type = other DNA
tgcgcttgcgcactgactcgggg

SEQ ID NO: 46 - DNA, 23 bp, synthetic construct
  source 1..23: mol_type = other DNA
gtccggttaagccacacctccgg

SEQ ID NO: 47 - DNA, 23 bp, synthetic construct
  source 1..23: mol_type = other DNA
ggacgcgagcagtgaaagcccgg

SEQ ID NO: 48 - DNA, 23 bp, synthetic construct
  source 1..23: mol_type = other DNA
cttaatctaagggaagttcctgg

SEQ ID NO: 49 - DNA, 23 bp, synthetic construct
  source 1..23: mol_type = other DNA
tccggtgggcgaactgagccagg

SEQ ID NO: 50 - DNA, 23 bp, synthetic construct
  source 1..23: mol_type = other DNA
ggacgcccagagcgtgtcgcggg

SEQ ID NO: 51 - DNA, 23 bp, synthetic construct
  source 1..23: mol_type = other DNA
aatgcgcttgcgcactgactcgg

SEQ ID NO: 52 - DNA, 23 bp, synthetic construct
  source 1..23: mol_type = other DNA
ttgaatgcccgcctcttctacgg

SEQ ID NO: 53 - DNA, 23 bp, synthetic construct
  source 1..23: mol_type = other DNA
atgcgcttgcgcactgactcggg

SEQ ID NO: 54 - DNA, 23 bp, synthetic construct
  source 1..23: mol_type = other DNA
gattatagcgattatatgagcgg

SEQ ID NO: 55 - DNA, 23 bp, synthetic construct
  source 1..23: mol_type = other DNA
cggcgtggaggatcggaaagagg

SEQ ID NO: 56 - DNA, 23 bp, synthetic construct
  source 1..23: mol_type = other DNA
attatagcgattatatgagcggg

SEQ ID NO: 57 - DNA, 23 bp, synthetic construct
  source 1..23: mol_type = other DNA
ctctttccgatcctccacgccgg

SEQ ID NO: 58 - DNA, 23 bp, synthetic construct
  source 1..23: mol_type = other DNA
aaagcccgggccgattccggtgg

SEQ ID NO: 59 - DNA, 23 bp, synthetic construct
  source 1..23: mol_type = other DNA
gcacttccggtcgggccctcggg

SEQ ID NO: 60 - DNA, 23 bp, synthetic construct
  source 1..23: mol_type = other DNA
attcaaagcgcaggcgcacctgg

SEQ ID NO: 61 - DNA, 23 bp, synthetic construct
  source 1..23: mol_type = other DNA
atcctccacgccggcgcctcggg

SEQ ID NO: 62 - DNA, 23 bp, synthetic construct
  source 1..23: mol_type = other DNA
gacttccgcttccgggcgggcgg

SEQ ID NO: 63 - DNA, 23 bp, synthetic construct
  source 1..23: mol_type = other DNA
ggcacttccggtcgggccctcgg

SEQ ID NO: 64 - DNA, 23 bp, synthetic construct
  source 1..23: mol_type = other DNA
agacgggagacccggcgccgggg

SEQ ID NO: 65 - DNA, 23 bp, synthetic construct
  source 1..23: mol_type = other DNA
gacgcgagcagtgaaagcccggg

SEQ ID NO: 66 - DNA, 23 bp, synthetic construct
  source 1..23: mol_type = other DNA
tcatataatcgctataatcccgg

SEQ ID NO: 67 - DNA, 23 bp, synthetic construct
  source 1..23: mol_type = other DNA
gcgcgacttccgcttccgggcgg

SEQ ID NO: 68 - DNA, 23 bp, synthetic construct
  source 1..23: mol_type = other DNA
atcacgctgtgagatgggcgtgg

SEQ ID NO: 69 - DNA, 23 bp, synthetic construct
  source 1..23: mol_type = other DNA
ctattccgcatggtctgagcagg

SEQ ID NO: 70 - DNA, 23 bp, synthetic construct
  source 1..23: mol_type = other DNA
accatgcggaatagaagccctgg

SEQ ID NO: 71 - DNA, 23 bp, synthetic construct
  source 1..23: mol_type = other DNA
caagcgcatttcccccctcctgg

SEQ ID NO: 72 - DNA, 23 bp, synthetic construct
  source 1..23: mol_type = other DNA
gtgaaagcccgggccgattccgg

SEQ ID NO: 73 - DNA, 23 bp, synthetic construct
  source 1..23: mol_type = other DNA
acgggcgcgacttccgcttccgg

SEQ ID NO: 74 - DNA, 23 bp, synthetic construct
  source 1..23: mol_type = other DNA
cagtgtgagacccgagccggagg

SEQ ID NO: 75 - DNA, 23 bp, synthetic construct
  source 1..23: mol_type = other DNA
agggggcggcacttccggtcggg

SEQ ID NO: 76 - DNA, 23 bp, synthetic construct
  source 1..23: mol_type = other DNA
acctggctcagttcgcccaccgg

SEQ ID NO: 77 - DNA, 23 bp, synthetic construct
  source 1..23: mol_type = other DNA
ttaagccacacctccggctcggg

SEQ ID NO: 78 - DNA, 23 bp, synthetic construct
  source 1..23: mol_type = other DNA
tgaatgcccgcctcttctacggg

SEQ ID NO: 79 - DNA, 23 bp, synthetic construct
  source 1..23: mol_type = other DNA
gccgctccggggagacccgaggg

SEQ ID NO: 80 - DNA, 23 bp, synthetic construct
  source 1..23: mol_type = other DNA
ggtgcaacttaaatgctatgggg

SEQ ID NO: 81 - DNA, 23 bp, synthetic construct
  source 1..23: mol_type = other DNA
aggtgcaacttaaatgctatggg

SEQ ID NO: 82 - DNA, 23 bp, synthetic construct
  source 1..23: mol_type = other DNA
cgggcgcgacttccgcttccggg

SEQ ID NO: 83 - DNA, 23 bp, synthetic construct
  source 1..23: mol_type = other DNA
gggaaccccgcgacacgctctgg

SEQ ID NO: 84 - DNA, 23 bp, synthetic construct
  source 1..23: mol_type = other DNA
gatcctccacgccggcgcctcgg

SEQ ID NO: 85 - DNA, 23 bp, synthetic construct
  source 1..23: mol_type = other DNA
cgcgacttccgcttccgggcggg

SEQ ID NO: 86 - DNA, 23 bp, synthetic construct
  source 1..23: mol_type = other DNA
catgcggaatagaagccctgggg

SEQ ID NO: 87 - DNA, 23 bp, synthetic construct
  source 1..23: mol_type = other DNA
acttaaatgctatggggtgaagg

SEQ ID NO: 88 - DNA, 23 bp, synthetic construct
  source 1..23: mol_type = other DNA
cggtggttgtcaaggatagttgg

SEQ ID NO: 89 - DNA, 23 bp, synthetic construct
  source 1..23: mol_type = other DNA
gagggggcggcacttccggtcgg

SEQ ID NO: 90 - DNA, 23 bp, synthetic construct
  source 1..23: mol_type = other DNA
gaggcgccggcgtggaggatcgg

SEQ ID NO: 91 - DNA, 23 bp, synthetic construct
  source 1..23: mol_type = other DNA
gggagacccgagggcccgaccgg

SEQ ID NO: 92 - DNA, 23 bp, synthetic construct
  source 1..23: mol_type = other DNA
actctgattctgctgcatgctgg

SEQ ID NO: 93 - DNA, 23 bp, synthetic construct
  source 1..23: mol_type = other DNA
gtcgggccctcgggtctccccgg

SEQ ID NO: 94 - DNA, 23 bp, synthetic construct
  source 1..23: mol_type = other DNA
cggaatagaagccctggggttgg

SEQ ID NO: 95 - DNA, 23 bp, synthetic construct
  source 1..23: mol_type = other DNA
cttataatcacgctgtgagatgg

SEQ ID NO: 96 - DNA, 23 bp, synthetic construct
  source 1..23: mol_type = other DNA
gagaaactgcatgagggcgaggg

SEQ ID NO: 97 - DNA, 23 bp, synthetic construct
  source 1..23: mol_type = other DNA
aaagcattcttttaaatcggtgg

SEQ ID NO: 98 - DNA, 23 bp, synthetic construct
  source 1..23: mol_type = other DNA
ctcgggtctccccggagcggcgg

SEQ ID NO: 99 - DNA, 23 bp, synthetic construct
  source 1..23: mol_type = other DNA
ggggacgaagacagactttgagg

SEQ ID NO: 100 - DNA, 23 bp, synthetic construct
  source 1..23: mol_type = other DNA
tgagacccgagccggaggtgtgg

SEQ ID NO: 101 - DNA, 23 bp, synthetic construct
  source 1..23: mol_type = other DNA
ggacttagaaggtgctgatgggg

SEQ ID NO: 102 - DNA, 23 bp, synthetic construct
  source 1..23: mol_type = other DNA
cgcgcccgttcccgaggcgccgg

SEQ ID NO: 103 - DNA, 23 bp, synthetic construct
  source 1..23: mol_type = other DNA
gagacgggagacccggcgccggg

SEQ ID NO: 104 - DNA, 23 bp, synthetic construct
  source 1..23: mol_type = other DNA
cgtggaggatcggaaagaggagg

SEQ ID NO: 105 - DNA, 23 bp, synthetic construct
  source 1..23: mol_type = other DNA
ttaatctaagggaagttcctggg

SEQ ID NO: 106 - DNA, 23 bp, synthetic construct
  source 1..23: mol_type = other DNA
cttagcaggaggggctgaatggg

SEQ ID NO: 107 - DNA, 23 bp, synthetic construct
  source 1..23: mol_type = other DNA
gaaggaagtacttaatctaaggg

SEQ ID NO: 108 - DNA, 23 bp, synthetic construct
  source 1..23: mol_type = other DNA
tgtcaagtgtggtaaatccatgg

SEQ ID NO: 109 - DNA, 23 bp, synthetic construct
  source 1..23: mol_type = other DNA
ttcccgaggcgccggcgtggagg

SEQ ID NO: 110 - DNA, 23 bp, synthetic construct
  source 1..23: mol_type = other DNA
gcttagcaggaggggctgaatgg

SEQ ID NO: 111 - DNA, 23 bp, synthetic construct
  source 1..23: mol_type = other DNA
tgctatggggtgaaggggcgggg

SEQ ID NO: 112 - DNA, 23 bp, synthetic construct
  source 1..23: mol_type = other DNA
ggggtgaagttccagagagtggg

SEQ ID NO: 113 - DNA, 23 bp, synthetic construct
  source 1..23: mol_type = other DNA
cgtggttcagggccgagggccgg

SEQ ID NO: 114 - DNA, 23 bp, synthetic construct
  source 1..23: mol_type = other DNA
cggggttcccgtagaagaggcgg

SEQ ID NO: 115 - DNA, 23 bp, synthetic construct
  source 1..23: mol_type = other DNA
gaggaggcgccgccgctccgggg

SEQ ID NO: 116 - DNA, 23 bp, synthetic construct
  source 1..23: mol_type = other DNA
actctgagggaacagggtattgg

SEQ ID NO: 117 - DNA, 23 bp, synthetic construct
  source 1..23: mol_type = other DNA
cttaaatgctatggggtgaaggg

SEQ ID NO: 118 - DNA, 23 bp, synthetic construct
  source 1..23: mol_type = other DNA
ttaaatgctatggggtgaagggg

SEQ ID NO: 119 - DNA, 23 bp, synthetic construct
  source 1..23: mol_type = other DNA
ctcctttaagccccggcccccgg

SEQ ID NO: 120 - DNA, 23 bp, synthetic construct
  source 1..23: mol_type = other DNA
ggccgggggccggggcttaaagg

SEQ ID NO: 121 - DNA, 23 bp, synthetic construct
  source 1..23: mol_type = other DNA
tgggcgtggttcagggccgaggg

SEQ ID NO: 122 - DNA, 23 bp, synthetic construct
  source 1..23: mol_type = other DNA
ggaggcactcaaaagtcacgagg

SEQ ID NO: 123 - DNA, 23 bp, synthetic construct
  source 1..23: mol_type = other DNA
gaggtgcaacttaaatgctatgg

SEQ ID NO: 124 - DNA, 23 bp, synthetic construct
  source 1..23: mol_type = other DNA
gggacttagaaggtgctgatggg

SEQ ID NO: 125 - DNA, 23 bp, synthetic construct
  source 1..23: mol_type = other DNA
cttcagtgtgagacccgagccgg

SEQ ID NO: 126 - DNA, 23 bp, synthetic construct
  source 1..23: mol_type = other DNA
ggagaaactgcatgagggcgagg

SEQ ID NO: 127 - DNA, 23 bp, synthetic construct
  source 1..23: mol_type = other DNA
tgggagagtggaagcttagcagg

SEQ ID NO: 128 - DNA, 23 bp, synthetic construct
  source 1..23: mol_type = other DNA
ggagacgggagacccggcgccgg

SEQ ID NO: 129 - DNA, 23 bp, synthetic construct
  source 1..23: mol_type = other DNA
gggctgaatgggtctgggtttgg

SEQ ID NO: 130 - DNA, 23 bp, synthetic construct
  source 1..23: mol_type = other DNA
agacagactttgaggcagtaggg

SEQ ID NO: 131 - DNA, 23 bp, synthetic construct
  source 1..23: mol_type = other DNA
tggttcagggccgagggccgggg

SEQ ID NO: 132 - DNA, 23 bp, synthetic construct
  source 1..23: mol_type = other DNA
gagagtggaagcttagcaggagg

SEQ ID NO: 133 - DNA, 23 bp, synthetic construct
  source 1..23: mol_type = other DNA
cgccgctccggggagacccgagg

SEQ ID NO: 134 - DNA, 23 bp, synthetic construct
  source 1..23: mol_type = other DNA
agaaggaagtacttaatctaagg

SEQ ID NO: 135 - DNA, 23 bp, synthetic construct
  source 1..23: mol_type = other DNA
atgctatggggtgaaggggcggg

SEQ ID NO: 136 - DNA, 23 bp, synthetic construct
  source 1..23: mol_type = other DNA
cacgactgctatggaggcgatgg

SEQ ID NO: 137 - DNA, 23 bp, synthetic construct
  source 1..23: mol_type = other DNA
ggcttaaaggagaaactctgagg

SEQ ID NO: 138 - DNA, 23 bp, synthetic construct
  source 1..23: mol_type = other DNA
ctgagggaacagggtattggagg

SEQ ID NO: 139 - DNA, 23 bp, synthetic construct
  source 1..23: mol_type = other DNA
atgggcgtggttcagggccaggg

SEQ ID NO: 140 - DNA, 23 bp, synthetic construct
  source 1..23: mol_type = other DNA
tgctaagcttccactctcccagg

SEQ ID NO: 141 - DNA, 23 bp, synthetic construct
  source 1..23: mol_type = other DNA
tctccgccgggaggaggccgagg

SEQ ID NO: 142 - DNA, 23 bp, synthetic construct
  source 1..23: mol_type = other DNA
gacttagaaggtgctgatggggg

SEQ ID NO: 143 - DNA, 23 bp, synthetic construct
  source 1..23: mol_type = other DNA
aagacagactttgaggcagtagg

SEQ ID NO: 144 - DNA, 23 bp, synthetic construct
  source 1..23: mol_type = other DNA
ttttaaatcggtggttgtcaagg

SEQ ID NO: 145 - DNA, 23 bp, synthetic construct
  source 1..23: mol_type = other DNA
aagaatgctttagaggtgggagg

SEQ ID NO: 146 - DNA, 23 bp, synthetic construct
  source 1..23: mol_type = other DNA
gatgggcgtggttcagggccagg

SEQ ID NO: 147 - DNA, 23 bp, synthetic construct
  source 1..23: mol_type = other DNA
ccggcggagaccccggcgccggg

SEQ ID NO: 148 - DNA, 23 bp, synthetic construct
  source 1..23: mol_type = other DNA
cctcctcccggcggagaccccgg

SEQ ID NO: 149 - DNA, 23 bp, synthetic construct
  source 1..23: mol_type = other DNA
ggggttcccgtagaagaggcggg

SEQ ID NO: 150 - DNA, 23 bp, synthetic construct
  source 1..23: mol_type = other DNA
gttgggaggccagcagcatccgg

SEQ ID NO: 151 - DNA, 23 bp, synthetic construct
  source 1..23: mol_type = other DNA
tgggacttagaaggtgctgatgg

SEQ ID NO: 152 - DNA, 23 bp, synthetic construct
  source 1..23: mol_type = other DNA
aatgctatggggtgaaggggcgg

SEQ ID NO: 153 - DNA, 23 bp, synthetic construct
  source 1..23: mol_type = other DNA
cgggtgggagacgggagacccgg

SEQ ID NO: 154 - DNA, 23 bp, synthetic construct
  source 1..23: mol_type = other DNA
tgggcgtggttcagggccagggg

SEQ ID NO: 155 - DNA, 23 bp, synthetic construct
  source 1..23: mol_type = other DNA
gtggttcagggccgagggccggg

SEQ ID NO: 156 - DNA, 23 bp, synthetic construct
  source 1..23: mol_type = other DNA
agagtggaagcttagcaggaggg

SEQ ID NO: 157 - DNA, 23 bp, synthetic construct
  source 1..23: mol_type = other DNA
ggttcagggccgagggccggggg

SEQ ID NO: 158 - DNA, 23 bp, synthetic construct
  source 1..23: mol_type = other DNA
aggagaaactctgagggaacagg

SEQ ID NO: 159 - DNA, 23 bp, synthetic construct
  source 1..23: mol_type = other DNA
gaacaggagaaactgcatgaggg

SEQ ID NO: 160 - DNA, 23 bp, synthetic construct
  source 1..23: mol_type = other DNA
tgggtttggctgtttgggatggg

SEQ ID NO: 161 - DNA, 23 bp, synthetic construct
  source 1..23: mol_type = other DNA
acagactttgaggcagtaggggg

SEQ ID NO: 162 - DNA, 23 bp, synthetic construct
  source 1..23: mol_type = other DNA
tgagcgggcagtgtcaagtgtgg

SEQ ID NO: 163 - DNA, 23 bp, synthetic construct
  source 1..23: mol_type = other DNA
ggtggttgtcaaggatagttggg

SEQ ID NO: 164 - DNA, 23 bp, synthetic construct
  source 1..23: mol_type = other DNA
cccggcggagaccccggcgccgg

SEQ ID NO: 165 - DNA, 23 bp, synthetic construct
  source 1..23: mol_type = other DNA
ctgggtttggctgtttgggatgg

SEQ ID NO: 166 - DNA, 23 bp, synthetic construct
  source 1..23: mol_type = other DNA
gtctcccgtctcccacccgccgg

SEQ ID NO: 167 - DNA, 23 bp, synthetic construct
  source 1..23: mol_type = other DNA
ctgtgagatgggcgtggttcagg

SEQ ID NO: 168 - DNA, 23 bp, synthetic construct
  source 1..23: mol_type = other DNA
gcgccggggtctccgccgggagg

SEQ ID NO: 169 - DNA, 23 bp, synthetic construct
  source 1..23: mol_type = other DNA
ggagaaactctgagggaacaggg

SEQ ID NO: 170 - DNA, 23 bp, synthetic construct
  source 1..23: mol_type = other DNA
cacgccggcgcctcgggaacggg

SEQ ID NO: 171 - DNA, 23 bp, synthetic construct
  source 1..23: mol_type = other DNA
agagtttctcctttaagccccgg

SEQ ID NO: 172 - DNA, 23 bp, synthetic construct
  source 1..23: mol_type = other DNA
cgtggttcagggccagggggcgg

SEQ ID NO: 173 - DNA, 23 bp, synthetic construct
  source 1..23: mol_type = other DNA
gtgggcgtggttcagggccgagg

SEQ ID NO: 174 - DNA, 23 bp, synthetic construct
  source 1..23: mol_type = other DNA
ctggaacttcacccctccctagg

SEQ ID NO: 175 - DNA, 23 bp, synthetic construct
  source 1..23: mol_type = other DNA
aggaggggctgaatgggtctggg

SEQ ID NO: 176 - DNA, 23 bp, synthetic construct
  source 1..23: mol_type = other DNA
ggaatagaagccctggggttggg

SEQ ID NO: 177 - DNA, 23 bp, synthetic construct
  source 1..23: mol_type = other DNA
tgcatgctggtcaagagccatgg

SEQ ID NO: 178 - DNA, 23 bp, synthetic construct
  source 1..23: mol_type = other DNA
gacagactttgaggcagtagggg

SEQ ID NO: 179 - DNA, 23 bp, synthetic construct
  source 1..23: mol_type = other DNA
ttataatcacgctgtgagatggg

SEQ ID NO: 180 - DNA, 23 bp, synthetic construct
  source 1..23: mol_type = other DNA
ccacgccggcgcctcgggaacgg

SEQ ID NO: 181 - DNA, 23 bp, synthetic construct
  source 1..23: mol_type = other DNA
cccagggcttctattccgcatgg

SEQ ID NO: 182 - DNA, 23 bp, synthetic construct
  source 1..23: mol_type = other DNA
tgggtctgggtttggctgtttgg

SEQ ID NO: 183 - DNA, 23 bp, synthetic construct
  source 1..23: mol_type = other DNA
gggcgtggttcagggccaggggg

SEQ ID NO: 184 - DNA, 23 bp, synthetic construct
  source 1..23: mol_type = other DNA
ggctgcctgctcagaccatgcgg

SEQ ID NO: 185 - DNA, 23 bp, synthetic construct
  source 1..23: mol_type = other DNA
agaaactgcatgagggcgagggg

SEQ ID NO: 186 - DNA, 23 bp, synthetic construct
  source 1..23: mol_type = other DNA
gagtggaagcttagcaggagggg

SEQ ID NO: 187 - DNA, 23 bp, synthetic construct
  source 1..23: mol_type = other DNA
taaaagaatgctttagaggtggg

SEQ ID NO: 188 - DNA, 23 bp, synthetic construct
  source 1..23: mol_type = other DNA
cggaaagaggaggcggtggcggg

SEQ ID NO: 189 - DNA, 23 bp, synthetic construct
  source 1..23: mol_type = other DNA
gggtctgggtttggctgtttggg

SEQ ID NO: 190 - DNA, 23 bp, synthetic construct
  source 1..23: mol_type = other DNA
cggaggaggcgccgccgctccgg

SEQ ID NO: 191 - DNA, 23 bp, synthetic construct
  source 1..23: mol_type = other DNA
gatttaaaagaatgctttagagg

SEQ ID NO: 192 - DNA, 23 bp, synthetic construct
  source 1..23: mol_type = other DNA
ccgcttccgggcgggcgggcagg

SEQ ID NO: 193 - DNA, 23 bp, synthetic construct
  source 1..23: mol_type = other DNA
tggttcagggccagggggcgggg

SEQ ID NO: 194 - DNA, 23 bp, synthetic construct
  source 1..23: mol_type = other DNA
aagggaagttcctgggagagtgg

SEQ ID NO: 195 - DNA, 23 bp, synthetic construct
  source 1..23: mol_type = other DNA
agttccagagagtgggagcttgg

SEQ ID NO: 196 - DNA, 23 bp, synthetic construct
  source 1..23: mol_type = other DNA
aatagaagccctggggttggggg

SEQ ID NO: 197 - DNA, 23 bp, synthetic construct
  source 1..23: mol_type = other DNA
aggggtgaagttccagagagtgg

SEQ ID NO: 198 - DNA, 23 bp, synthetic construct
  source 1..23: mol_type = other DNA
gggtttggctgtttgggatgggg

SEQ ID NO: 199 - DNA, 23 bp, synthetic construct
  source 1..23: mol_type = other DNA
gccctcgggtctccccggagcgg

SEQ ID NO: 200 - DNA, 23 bp, synthetic construct
  source 1..23: mol_type = other DNA
aatcagagtatttcaaaacacgg

SEQ ID NO: 201 - DNA, 23 bp, synthetic construct
  source 1..23: mol_type = other DNA
tctaaagcattcttttaaatcgg

SEQ ID NO: 202 - DNA, 23 bp, synthetic construct
  source 1..23: mol_type = other DNA
gcttaaaggagaaactctgaggg

SEQ ID NO: 203 - DNA, 23 bp, synthetic construct
  source 1..23: mol_type = other DNA
gggggagggggcggcacttccgg

SEQ ID NO: 204 - DNA, 23 bp, synthetic construct
  source 1..23: mol_type = other DNA
gaatagaagccctggggttgggg

SEQ ID NO: 205 - DNA, 23 bp, synthetic construct
  source 1..23: mol_type = other DNA
agggaacagggtattggaggtgg

SEQ ID NO: 206 - DNA, 23 bp, synthetic construct
  source 1..23: mol_type = other DNA
agaacaggagaaactgcatgagg

SEQ ID NO: 207 - DNA, 23 bp, synthetic construct
  source 1..23: mol_type = other DNA
gggaacagggtattggaggtggg

SEQ ID NO: 208 - DNA, 23 bp, synthetic construct
  source 1..23: mol_type = other DNA
gcatggtctgagcaggcagccgg

SEQ ID NO: 209 - DNA, 23 bp, synthetic construct
  source 1..23: mol_type = other DNA
ggatcggaaagaggaggcggtgg

SEQ ID NO: 210 - DNA, 23 bp, synthetic construct
  source 1..23: mol_type = other DNA
agcaggcagccggatgctgctgg

SEQ ID NO: 211 - DNA, 23 bp, synthetic construct
  source 1..23: mol_type = other DNA
caggaggggctgaatgggtctgg

SEQ ID NO: 212 - DNA, 23 bp, synthetic construct
  source 1..23: mol_type = other DNA
atccctagacccccaaccccagg

SEQ ID NO: 213 - DNA, 23 bp, synthetic construct
  source 1..23: mol_type = other DNA
gatggagggggcggggcctaggg

SEQ ID NO: 214 - DNA, 23 bp, synthetic construct
  source 1..23: mol_type = other DNA
ttaaaagaatgctttagaggtgg

SEQ ID NO: 215 - DNA, 23 bp, synthetic construct
  source 1..23: mol_type = other DNA
cacactgaagccccgccccctgg

SEQ ID NO: 216 - DNA, 23 bp, synthetic construct
  source 1..23: mol_type = other DNA
tcggaaagaggaggcggtggcgg

SEQ ID NO: 217 - DNA, 23 bp, synthetic construct
  source 1..23: mol_type = other DNA
tgtgagatgggcgtggttcaggg

SEQ ID NO: 218 - DNA, 23 bp, synthetic construct
  source 1..23: mol_type = other DNA
gccctggggttgggggtctaggg

SEQ ID NO: 219 - DNA, 23 bp, synthetic construct
  source 1..23: mol_type = other DNA
gcttggagagggggaagaacagg

SEQ ID NO: 220 - DNA, 23 bp, synthetic construct
  source 1..23: mol_type = other DNA
gcggtggcggggaccaggagggg

SEQ ID NO: 221 - DNA, 23 bp, synthetic construct
  source 1..23: mol_type = other DNA
cggtggcggggaccaggaggggg

SEQ ID NO: 222 - DNA, 23 bp, synthetic construct
  source 1..23: mol_type = other DNA
ggaggaggcgccgccgctccggg

SEQ ID NO: 223 - DNA, 23 bp, synthetic construct
  source 1..23: mol_type = other DNA
tgtttgggatggggctgagttgg

SEQ ID NO: 224 - DNA, 23 bp, synthetic construct
  source 1..23: mol_type = other DNA
aaggggcggggcttggatggagg

SEQ ID NO: 225 - DNA, 23 bp, synthetic construct
  source 1..23: mol_type = other DNA
gtttgggatggggctgagttggg

SEQ ID NO: 226 - DNA, 23 bp, synthetic construct
  source 1..23: mol_type = other DNA
gtgaaggggcggggcttggatgg

SEQ ID NO: 227 - DNA, 23 bp, synthetic construct
  source 1..23: mol_type = other DNA
aagccccggcccccggccctcgg

SEQ ID NO: 228 - DNA, 23 bp, synthetic construct
  source 1..23: mol_type = other DNA
agagagtgggagcttggagaggg

SEQ ID NO: 229 - DNA, 23 bp, synthetic construct
  source 1..23: mol_type = other DNA
gtggttcagggccagggggcggg

SEQ ID NO: 230 - DNA, 23 bp, synthetic construct
  source 1..23: mol_type = other DNA
cttccgggcgggcgggcaggcgg

SEQ ID NO: 231 - DNA, 23 bp, synthetic construct
  source 1..23: mol_type = other DNA
cctcctcggcctcctcccggcgg

SEQ ID NO: 232 - DNA, 23 bp, synthetic construct
  source 1..23: mol_type = other DNA
atggggctgagttgggagccggg

SEQ ID NO: 233 - DNA, 23 bp, synthetic construct
  source 1..23: mol_type = other DNA
aggcggtggcggggaccaggagg

SEQ ID NO: 234 - DNA, 23 bp, synthetic construct
  source 1..23: mol_type = other DNA
ggaaagaggaggcggtggcgggg

SEQ ID NO: 235 - DNA, 23 bp, synthetic construct
  source 1..23: mol_type = other DNA
cgggcggtgcggggctgccgggg

SEQ ID NO: 236 - DNA, 23 bp, synthetic construct
  source 1..23: mol_type = other DNA
ggcggtggcggggaccaggaggg

SEQ ID NO: 237 - DNA, 23 bp, synthetic construct
  source 1..23: mol_type = other DNA
agccctggggttgggggtctagg

SEQ ID NO: 238 - DNA, 23 bp, synthetic construct
  source 1..23: mol_type = other DNA
atggaggcgatggggagagaagg

SEQ ID NO: 239 - DNA, 23 bp, synthetic construct
  source 1..23: mol_type = other DNA
tccctagacccccaaccccaggg

SEQ ID NO: 240 - DNA, 23 bp, synthetic construct
  source 1..23: mol_type = other DNA
cagagagtgggagcttggagagg

SEQ ID NO: 241 - DNA, 23 bp, synthetic construct
  source 1..23: mol_type = other DNA
aggggcggggcttggatggaggg

SEQ ID NO: 242 - DNA, 23 bp, synthetic construct
  source 1..23: mol_type = other DNA
cggtgcggggctgccggggaggg

SEQ ID NO: 243 - DNA, 23 bp, synthetic construct
  source 1..23: mol_type = other DNA
cggggcttggatggagggggcgg

SEQ ID NO: 244 - DNA, 23 bp, synthetic construct
  source 1..23: mol_type = other DNA
gcggtgcggggctgccggggagg

SEQ ID NO: 245 - DNA, 23 bp, synthetic construct
  source 1..23: mol_type = other DNA
gatggggctgagttgggagccgg

SEQ ID NO: 246 - DNA, 23 bp, synthetic construct
  source 1..23: mol_type = other DNA
ctctccaagctcccactctctgg

SEQ ID NO: 247 - DNA, 23 bp, synthetic construct
  source 1..23: mol_type = other DNA
gcgggcggtgcggggctgccggg

SEQ ID NO: 248 - DNA, 23 bp, synthetic construct
  source 1..23: mol_type = other DNA
gagagtgggagcttggagagggg

SEQ ID NO: 249 - DNA, 23 bp, synthetic construct
  source 1..23: mol_type = other DNA
tgcggggctgccggggagggggg

SEQ ID NO: 250 - DNA, 23 bp, synthetic construct
  source 1..23: mol_type = other DNA
gggcttggatggagggggcgggg

SEQ ID NO: 251 - DNA, 23 bp, synthetic construct
  source 1..23: mol_type = other DNA
ggtggcggggaccaggagggggg

SEQ ID NO: 252 - DNA, 23 bp, synthetic construct
  source 1..23: mol_type = other DNA
agagtgggagcttggagaggggg

SEQ ID NO: 253 - DNA, 23 bp, synthetic construct
  source 1..23: mol_type = other DNA
ttccgggcgggcgggcaggcggg

SEQ ID NO: 254 - DNA, 23 bp, synthetic construct
  source 1..23: mol_type = other DNA
gggcggggcttggatggaggggg

SEQ ID NO: 255 - DNA, 23 bp, synthetic construct
  source 1..23: mol_type = other DNA
aggaggcggtggcggggaccagg

SEQ ID NO: 256 - DNA, 23 bp, synthetic construct
  source 1..23: mol_type = other DNA
ggggcggggcttggatggagggg

SEQ ID NO: 257 - DNA, 23 bp, synthetic construct
  source 1..23: mol_type = other DNA
tggggtgaaggggcggggcttgg

SEQ ID NO: 258 - DNA, 23 bp, synthetic construct
  source 1..23: mol_type = other DNA
ggcgggcggtgcggggctgccgg

SEQ ID NO: 259 - DNA, 23 bp, synthetic construct
  source 1..23: mol_type = other DNA
ggttgtcaaggatagttgggagg

SEQ ID NO: 260 - DNA, 23 bp, synthetic construct
  source 1..23: mol_type = other DNA
ggggcttggatggagggggcggg

SEQ ID NO: 261 - DNA, 23 bp, synthetic construct
  source 1..23: mol_type = other DNA
gcgggcaggcgggcggtgcgggg

SEQ ID NO: 262 - DNA, 23 bp, synthetic construct
  source 1..23: mol_type = other DNA
gtgcggggctgccggggaggggg

SEQ ID NO: 263 - DNA, 23 bp, synthetic construct
  source 1..23: mol_type = other DNA
ggtgcggggctgccggggagggg

SEQ ID NO: 264 - DNA, 23 bp, synthetic construct
  source 1..23: mol_type = other DNA
ccgcctcctcggcctcctcccgg

SEQ ID NO: 265 - DNA, 23 bp, synthetic construct
  source 1..23: mol_type = other DNA
cggcgcctcctccgcctcctcgg

SEQ ID NO: 266 - DNA, 23 bp, synthetic construct
  source 1..23: mol_type = other DNA
ggatggagggggcggggcctagg

SEQ ID NO: 267 - DNA, 23 bp, synthetic construct
  source 1..23: mol_type = other DNA
tattggaggtgggacttagaagg

SEQ ID NO: 268 - DNA, 23 bp, synthetic construct
  source 1..23: mol_type = other DNA
agggggcggggcctagggagggg

SEQ ID NO: 269 - DNA, 23 bp, synthetic construct
  source 1..23: mol_type = other DNA
gagggggcggggcctagggaggg

SEQ ID NO: 270 - DNA, 23 bp, synthetic construct
  source 1..23: mol_type = other DNA
agggccgagggccgggggccggg

SEQ ID NO: 271 - DNA, 23 bp, synthetic construct
  source 1..23: mol_type = other DNA
ggcgggcaggcgggcggtgcggg

SEQ ID NO: 272 - DNA, 23 bp, synthetic construct
  source 1..23: mol_type = other DNA
cagggccgagggccgggggccgg

SEQ ID NO: 273 - DNA, 23 bp, synthetic construct
  source 1..23: mol_type = other DNA
ggctgccggggaggggggagggg

SEQ ID NO: 274 - DNA, 23 bp, synthetic construct
  source 1..23: mol_type = other DNA
gggcgggcaggcgggcggtgcgg

SEQ ID NO: 275 - DNA, 23 bp, synthetic construct
  source 1..23: mol_type = other DNA
gccgccccctcccccctccccgg

SEQ ID NO: 276 - DNA, 23 bp, synthetic construct
  source 1..23: mol_type = other DNA
ggagggggcggggcctagggagg

SEQ ID NO: 277 - DNA, 23 bp, synthetic construct
  source 1..23: mol_type = other DNA
gctgccggggaggggggaggggg

SEQ ID NO: 278 - DNA, 23 bp, synthetic construct
  source 1..23: mol_type = other DNA
gggctgccggggaggggggaggg

SEQ ID NO: 279 - DNA, 23 bp, synthetic construct
  source 1..23: mol_type = other DNA
ggaggatcggaaagaggaggcgg

SEQ ID NO: 280 - DNA, 23 bp, synthetic construct
  source 1..23: mol_type = other DNA
ggggctgccggggaggggggagg

SEQ ID NO: 281 - DNA, 23 bp, synthetic construct
  source 1..23: mol_type = other DNA
ggaggaggccgaggaggcggagg

SEQ ID NO: 282 - DNA, 23 bp, synthetic construct
  source 1..23: mol_type = other DNA
gccggggaggggggagggggcgg

SEQ ID NO: 283 - DNA, 23 bp, synthetic construct
  source 1..23: mol_type = other DNA
ggaggccgaggaggcggaggagg

SEQ ID NO: 284 - DNA, 23 bp, synthetic construct
  source 1..23: mol_type = other DNA
cactccgtacatagaaggtatgg

SEQ ID NO: 285 - DNA, 23 bp, synthetic construct
  source 1..23: mol_type = other DNA
ccgcacaaggatggatgagtggg

SEQ ID NO: 286 - DNA, 23 bp, synthetic construct
  source 1..23: mol_type = other DNA
tatcagcgtaattaggagggagg

SEQ ID NO: 287 - DNA, 23 bp, synthetic construct
  source 1..23: mol_type = other DNA
tttctatcagcgtaattaggagg

SEQ ID NO: 288 - DNA, 23 bp, synthetic construct
  source 1..23: mol_type = other DNA
cgtacatagaaggtatggactgg

SEQ ID NO: 289 - DNA, 23 bp, synthetic construct
  source 1..23: mol_type = other DNA
ggctcttagggggcggttcctgg

SEQ ID NO: 290 - DNA, 23 bp, synthetic construct
  source 1..23: mol_type = other DNA
tccgcacaaggatggatgagtgg

SEQ ID NO: 291 - DNA, 23 bp, synthetic construct
  source 1..23: mol_type = other DNA
acaaataatagagggcctgcagg

SEQ ID NO: 292 - DNA, 23 bp, synthetic construct
  source 1..23: mol_type = other DNA
cagtccataccttctatgtacgg

SEQ ID NO: 293 - DNA, 23 bp, synthetic construct
  source 1..23: mol_type = other DNA
cccctctacaaagctgtgtcagg

SEQ ID NO: 294 - DNA, 23 bp, synthetic construct
  source 1..23: mol_type = other DNA
ttctatcagcgtaattaggaggg

SEQ ID NO: 295 - DNA, 23 bp, synthetic construct
  source 1..23: mol_type = other DNA
gtaagcactccgtacatagaagg

SEQ ID NO: 296 - DNA, 23 bp, synthetic construct
  source 1..23: mol_type = other DNA
cagtttctatcagcgtaattagg

SEQ ID NO: 297 - DNA, 23 bp, synthetic construct
  source 1..23: mol_type = other DNA
caattgtccaagcatcctggggg

SEQ ID NO: 298 - DNA, 23 bp, synthetic construct
  source 1..23: mol_type = other DNA
gtcctgacacagctttgtagagg

SEQ ID NO: 299 - DNA, 23 bp, synthetic construct
  source 1..23: mol_type = other DNA
atatgtgcaaggctcttaggggg

SEQ ID NO: 300 - DNA, 23 bp, synthetic construct
  source 1..23: mol_type = other DNA
acatatgtgcaaggctcttaggg

SEQ ID NO: 301 - DNA, 23 bp, synthetic construct
  source 1..23: mol_type = other DNA
ctccaattgtccaagcatcctgg

SEQ ID NO: 302 - DNA, 23 bp, synthetic construct
  source 1..23: mol_type = other DNA
cacatatgtgcaaggctcttagg

SEQ ID NO: 303 - DNA, 23 bp, synthetic construct
  source 1..23: mol_type = other DNA
gcctaaatctggaaggctagggg

SEQ ID NO: 304 - DNA, 23 bp, synthetic construct
  source 1..23: mol_type = other DNA
gcagggcttacatttgattctgg

SEQ ID NO: 305 - DNA, 23 bp, synthetic construct
  source 1..23: mol_type = other DNA
aataatagagggcctgcaggtgg

SEQ ID NO: 306 - DNA, 23 bp, synthetic construct
  source 1..23: mol_type = other DNA
catatgtgcaaggctcttagggg

SEQ ID NO: 307 - DNA, 23 bp, synthetic construct
  source 1..23: mol_type = other DNA
gctagggggtgatccgcacaagg

SEQ ID NO: 308 - DNA, 23 bp, synthetic construct
  source 1..23: mol_type = other DNA
agcctaaatctggaaggctaggg

SEQ ID NO: 309 - DNA, 23 bp, synthetic construct
  source 1..23: mol_type = other DNA
tgaggacacggccgcggcgctgg

SEQ ID NO: 310 - DNA, 23 bp, synthetic construct
  source 1..23: mol_type = other DNA
tgcggggagacccagcgccgcgg

SEQ ID NO: 311 - DNA, 23 bp, synthetic construct
  source 1..23: mol_type = other DNA
gtcaggaccccagaaaagatggg

SEQ ID NO: 312 - DNA, 23 bp, synthetic construct
  source 1..23: mol_type = other DNA
taattaggagggaggactctggg

SEQ ID NO: 313 - DNA, 23 bp, synthetic construct
  source 1..23: mol_type = other DNA
cctaaatctggaaggctaggggg

SEQ ID NO: 314 - DNA, 23 bp, synthetic construct
  source 1..23: mol_type = other DNA
actggaagcctaaatctggaagg

SEQ ID NO: 315 - DNA, 23 bp, synthetic construct
  source 1..23: mol_type = other DNA
tgggtctccccgcaggtgaatgg

SEQ ID NO: 316 - DNA, 23 bp, synthetic construct
  source 1..23: mol_type = other DNA
agggactggaagcctaaatctgg

SEQ ID NO: 317 - skipped

SEQ ID NO: 318 - DNA, 23 bp, synthetic construct
  source 1..23: mol_type = other DNA
gggggtgatccgcacaaggatgg

SEQ ID NO: 319 - DNA, 23 bp, synthetic construct
  source 1..23: mol_type = other DNA
aagcctaaatctggaaggctagg

SEQ ID NO: 320 - DNA, 23 bp, synthetic construct
  source 1..23: mol_type = other DNA
tccaattgtccaagcatcctggg

SEQ ID NO: 321 - DNA, 23 bp, synthetic construct
  source 1..23: mol_type = other DNA
gaggacacggccgcggcgctggg

SEQ ID NO: 322 - DNA, 23 bp, synthetic construct
  source 1..23: mol_type = other DNA
gtacatagaaggtatggactggg

SEQ ID NO: 323 - DNA, 23 bp, synthetic construct
  source 1..23: mol_type = other DNA
tgtgcaaggctcttagggggcgg

SEQ ID NO: 324 - DNA, 23 bp, synthetic construct
  source 1..23: mol_type = other DNA
tcctgacacagctttgtagaggg

SEQ ID NO: 325 - DNA, 23 bp, synthetic construct
  source 1..23: mol_type = other DNA
agagaaagtagagacggcccagg

SEQ ID NO: 326 - DNA, 23 bp, synthetic construct
  source 1..23: mol_type = other DNA
tggacaattggagtctgaaaagg

SEQ ID NO: 327 - DNA, 23 bp, synthetic construct
  source 1..23: mol_type = other DNA
ggctgccctttggggtggacagg

SEQ ID NO: 328 - DNA, 23 bp, synthetic construct
  source 1..23: mol_type = other DNA
ccccaggatgcttggacaattgg

SEQ ID NO: 329 - DNA, 23 bp, synthetic construct
  source 1..23: mol_type = other DNA
aaggtgagtaggagagtcctggg

SEQ ID NO: 330 - DNA, 23 bp, synthetic construct
  source 1..23: mol_type = other DNA
cacagctccattcacctgcgggg

SEQ ID NO: 331 - DNA, 23 bp, synthetic construct
  source 1..23: mol_type = other DNA
gaaggtgagtaggagagtcctgg

SEQ ID NO: 332 - DNA, 23 bp, synthetic construct
  source 1..23: mol_type = other DNA
aaagcacatgcctacctcacagg

SEQ ID NO: 333 - DNA, 23 bp, synthetic construct
  source 1..23: mol_type = other DNA
aattaggagggaggactctgggg

SEQ ID NO: 334 - DNA, 23 bp, synthetic construct
  source 1..23: mol_type = other DNA
cgggagaccctgtgaagaggagg

SEQ ID NO: 335 - DNA, 23 bp, synthetic construct
  source 1..23: mol_type = other DNA
gtggtccccaggtgtagatgagg

SEQ ID NO: 336 - DNA, 23 bp, synthetic construct
  source 1..23: mol_type = other DNA
cattttaggataaaacctgaagg

SEQ ID NO: 337 - DNA, 23 bp, synthetic construct
  source 1..23: mol_type = other DNA
aggtgagtaggagagtcctgggg

SEQ ID NO: 338 - DNA, 23 bp, synthetic construct
  source 1..23: mol_type = other DNA
gccccgtcctcctcttcacaggg

SEQ ID NO: 339 - DNA, 23 bp, synthetic construct
  source 1..23: mol_type = other DNA
gcacagctccattcacctgcggg

SEQ ID NO: 340 - DNA, 23 bp, synthetic construct
  source 1..23: mol_type = other DNA
gtaattaggagggaggactctgg

SEQ ID NO: 341 - DNA, 23 bp, synthetic construct
  source 1..23: mol_type = other DNA
aagaggaagggctgccctttggg

SEQ ID NO: 342 - DNA, 23 bp, synthetic construct
  source 1..23: mol_type = other DNA
acctgaagggatgaggacggagg

SEQ ID NO: 343 - DNA, 23 bp, synthetic construct
  source 1..23: mol_type = other DNA
agacgggagaccctgtgaagagg

SEQ ID NO: 344 - DNA, 23 bp, synthetic construct
  source 1..23: mol_type = other DNA
aactgagtccagaggcccccagg

SEQ ID NO: 345 - DNA, 23 bp, synthetic construct
  source 1..23: mol_type = other DNA
cggggctgaggacacggccgcgg

SEQ ID NO: 346 - DNA, 23 bp, synthetic construct
  source 1..23: mol_type = other DNA
gtctcccgtctcccacccgccgg

SEQ ID NO: 347 - DNA, 23 bp, synthetic construct
  source 1..23: mol_type = other DNA
agccccgtcctcctcttcacagg

SEQ ID NO: 348 - DNA, 23 bp, synthetic construct
  source 1..23: mol_type = other DNA
accctgtgaagaggaggacgggg

SEQ ID NO: 349 - DNA, 23 bp, synthetic construct
  source 1..23: mol_type = other DNA
tgaggtaggcatgtgctttgtgg

SEQ ID NO: 350 - DNA, 23 bp, synthetic construct
  source 1..23: mol_type = other DNA
tgtcaggaccccagaaaagatgg

SEQ ID NO: 351 - DNA, 23 bp, synthetic construct
  source 1..23: mol_type = other DNA
cccgtttctacaaataatagagg

SEQ ID NO: 352 - DNA, 23 bp, synthetic construct
  source 1..23: mol_type = other DNA
tcaggaccccagaaaagatgggg

SEQ ID NO: 353 - DNA, 23 bp, synthetic construct
  source 1..23: mol_type = other DNA
ggatgaggacggagggctgtggg

SEQ ID NO: 354 - DNA, 23 bp, synthetic construct
  source 1..23: mol_type = other DNA
caagaggaagggctgccctttgg

SEQ ID NO: 355 - DNA, 23 bp, synthetic construct
  source 1..23: mol_type = other DNA
gcatgtgctttgtggtccccagg

SEQ ID NO: 356 - DNA, 23 bp, synthetic construct
  source 1..23: mol_type = other DNA
gtaggagagtcctggggccaggg

SEQ ID NO: 357 - DNA, 23 bp, synthetic construct
  source 1..23: mol_type = other DNA
gaccctgtgaagaggaggacggg

SEQ ID NO: 358 - DNA, 23 bp, synthetic construct
  source 1..23: mol_type = other DNA
cctgaagggatgaggacggaggg

SEQ ID NO: 359 - DNA, 23 bp, synthetic construct
  source 1..23: mol_type = other DNA
tagagggcctgcaggtggtgtgg

SEQ ID NO: 360 - DNA, 23 bp, synthetic construct
  source 1..23: mol_type = other DNA
cttatcattatgctcattttagg

SEQ ID NO: 361 - DNA, 23 bp, synthetic construct
  source 1..23: mol_type = other DNA
ccgtttctacaaataatagaggg

SEQ ID NO: 362 - DNA, 23 bp, synthetic construct
  source 1..23: mol_type = other DNA
tcaacacacacatatgtgcaagg

SEQ ID NO: 363 - DNA, 23 bp, synthetic construct
  source 1..23: mol_type = other DNA
agaggaagggctgccctttgggg

SEQ ID NO: 364 - DNA, 23 bp, synthetic construct
  source 1..23: mol_type = other DNA
atctctccccatcttttctgggg

SEQ ID NO: 365 - DNA, 23 bp, synthetic construct
  source 1..23: mol_type = other DNA
catctctccccatcttttctggg

SEQ ID NO: 366 - DNA, 23 bp, synthetic construct
  source 1..23: mol_type = other DNA
catttcctcatctacacctgggg

SEQ ID NO: 367 - DNA, 23 bp, synthetic construct
  source 1..23: mol_type = other DNA
cgtggccacacagctagaagtgg

SEQ ID NO: 368 - DNA, 23 bp, synthetic construct
  source 1..23: mol_type = other DNA
cctttggggtggacaggcagagg

SEQ ID NO: 369 - DNA, 23 bp, synthetic construct
  source 1..23: mol_type = other DNA
ccagaggcccccaggatgcttgg

SEQ ID NO: 370 - DNA, 23 bp, synthetic construct
  source 1..23: mol_type = other DNA
ctttggggtggacaggcagaggg

SEQ ID NO: 371 - DNA, 23 bp, synthetic construct
  source 1..23: mol_type = other DNA
ctctcctccacaccacctgcagg

SEQ ID NO: 372 - DNA, 23 bp, synthetic construct
  source 1..23: mol_type = other DNA
aaaatgagcataatgataagggg

SEQ ID NO: 373 - DNA, 23 bp, synthetic construct
  source 1..23: mol_type = other DNA
cggagtgcttacagagtgccagg

SEQ ID NO: 374 - DNA, 23 bp, synthetic construct
  source 1..23: mol_type = other DNA
aaaacctgaagggatgaggacgg

SEQ ID NO: 375 - DNA, 23 bp, synthetic construct
  source 1..23: mol_type = other DNA
gcagagggtctgagagggtttgg

SEQ ID NO: 376 - DNA, 23 bp, synthetic construct
  source 1..23: mol_type = other DNA
gggagttgagaacacagagaggg

SEQ ID NO: 377 - DNA, 23 bp, synthetic construct
  source 1..23: mol_type = other DNA
ggaagggctgccctttggggtgg

SEQ ID NO: 378 - DNA, 23 bp, synthetic construct
  source 1..23: mol_type = other DNA
caaagggcagcccttcctcttgg

SEQ ID NO: 379 - DNA, 23 bp, synthetic construct
  source 1..23: mol_type = other DNA
ccacacagctagaagtggcaggg

SEQ ID NO: 380 - DNA, 23 bp, synthetic construct
  source 1..23: mol_type = other DNA
tgggagttgagaacacagagagg

SEQ ID NO: 381 - DNA, 23 bp, synthetic construct
  source 1..23: mol_type = other DNA
agtaggagagtcctggggccagg

SEQ ID NO: 382 - DNA, 23 bp, synthetic construct
  source 1..23: mol_type = other DNA
cccaggtgtagatgaggaaatgg

SEQ ID NO: 383 - DNA, 23 bp, synthetic construct
  source 1..23: mol_type = other DNA
gttgagaacacagagagggaagg

SEQ ID NO: 384 - DNA, 23 bp, synthetic construct
  source 1..23: mol_type = other DNA
tgaatcctcacagtcctgtgagg

SEQ ID NO: 385 - DNA, 23 bp, synthetic construct
  source 1..23: mol_type = other DNA
agaccctgtgaagaggaggacgg

SEQ ID NO: 386 - DNA, 23 bp, synthetic construct
  source 1..23: mol_type = other DNA
gagtatggagagacagcagaggg

SEQ ID NO: 387 - DNA, 23 bp, synthetic construct
  source 1..23: mol_type = other DNA
tccatttcctcatctacacctgg

SEQ ID NO: 388 - DNA, 23 bp, synthetic construct
  source 1..23: mol_type = other DNA
gccacacagctagaagtggcagg

SEQ ID NO: 389 - DNA, 23 bp, synthetic construct
  source 1..23: mol_type = other DNA
agcccttcctcttggctccctgg

SEQ ID NO: 390 - DNA, 23 bp, synthetic construct
  source 1..23: mol_type = other DNA
tggggtagggtgcaggagtatgg

SEQ ID NO: 391 - DNA, 23 bp, synthetic construct
  source 1..23: mol_type = other DNA
gaggacggagggctgtgggtggg

SEQ ID NO: 392 - DNA, 23 bp, synthetic construct
  source 1..23: mol_type = other DNA
gagacagcagagggtctgagagg

SEQ ID NO: 393 - DNA, 23 bp, synthetic construct
  source 1..23: mol_type = other DNA
ggccagggagccaagaggaaggg

SEQ ID NO: 394 - DNA, 23 bp, synthetic construct
  source 1..23: mol_type = other DNA
ggagtatggagagacagcagagg

SEQ ID NO: 395 - DNA, 23 bp, synthetic construct
  source 1..23: mol_type = other DNA
tgaggacggagggctgtgggtgg

SEQ ID NO: 396 - DNA, 23 bp, synthetic construct
  source 1..23: mol_type = other DNA
ctctgcctgtccaccccaaaggg

SEQ ID NO: 397 - DNA, 23 bp, synthetic construct
  source 1..23: mol_type = other DNA
gggatgaggacggagggctgtgg

SEQ ID NO: 398 - DNA, 23 bp, synthetic construct
  source 1..23: mol_type = other DNA
gcctacctcacaggactgtgagg

SEQ ID NO: 399 - DNA, 23 bp, synthetic construct
  source 1..23: mol_type = other DNA
ggaaagaaaactgagtccagagg

SEQ ID NO: 400 - DNA, 23 bp, synthetic construct
  source 1..23: mol_type = other DNA
gggccagggagccaagaggaagg

SEQ ID NO: 401 - DNA, 23 bp, synthetic construct
  source 1..23: mol_type = other DNA
ctgtgggtggggtagggtgcagg

SEQ ID NO: 402 - DNA, 23 bp, synthetic construct
  source 1..23: mol_type = other DNA
gggtggacaggcagagggactgg

SEQ ID NO: 403 - DNA, 23 bp, synthetic construct
  source 1..23: mol_type = other DNA
ctaaaatgagcataatgataagg

SEQ ID NO: 404 - DNA, 23 bp, synthetic construct
  source 1..23: mol_type = other DNA
ggaggacggggctgaggacacgg

SEQ ID NO: 405 - DNA, 23 bp, synthetic construct
  source 1..23: mol_type = other DNA
agacagcagagggtctgagaggg

SEQ ID NO: 406 - DNA, 23 bp, synthetic construct
  source 1..23: mol_type = other DNA
taaaatgagcataatgataaggg

SEQ ID NO: 407 - DNA, 23 bp, synthetic construct
  source 1..23: mol_type = other DNA
attttaggataaaacctgaaggg

SEQ ID NO: 408 - DNA, 23 bp, synthetic construct
  source 1..23: mol_type = other DNA
acttctagctgtgtggccacggg

SEQ ID NO: 409 - DNA, 23 bp, synthetic construct
  source 1..23: mol_type = other DNA
attagagaaggaaaagaggtggg

SEQ ID NO: 410 - DNA, 23 bp, synthetic construct
  source 1..23: mol_type = other DNA
ctgcaggtggtgtggaggagagg

SEQ ID NO: 411 - DNA, 23 bp, synthetic construct
  source 1..23: mol_type = other DNA
aggacggagggctgtgggtgggg

SEQ ID NO: 412 - DNA, 23 bp, synthetic construct
  source 1..23: mol_type = other DNA
agggcctgcaggtggtgtggagg

SEQ ID NO: 413 - DNA, 23 bp, synthetic construct
  source 1..23: mol_type = other DNA
tgcaggtggtgtggaggagaggg

SEQ ID NO: 414 - DNA, 23 bp, synthetic construct
  source 1..23: mol_type = other DNA
ccagaaaagatggggagagatgg

SEQ ID NO: 415 - DNA, 23 bp, synthetic construct
  source 1..23: mol_type = other DNA
aggtgtagatgaggaaatggagg

SEQ ID NO: 416 - DNA, 23 bp, synthetic construct
  source 1..23: mol_type = other DNA
aacacagagagggaaggtgaagg

SEQ ID NO: 417 - DNA, 23 bp, synthetic construct
  source 1..23: mol_type = other DNA
gagggaaggtgaaggtgagtagg

SEQ ID NO: 418 - DNA, 23 bp, synthetic construct
  source 1..23: mol_type = other DNA
gtgagccagcgcgctggcctggg

SEQ ID NO: 419 - DNA, 23 bp, synthetic construct
  source 1..23: mol_type = other DNA
aattagagaaggaaaagaggtgg

SEQ ID NO: 420 - DNA, 23 bp, synthetic construct
  source 1..23: mol_type = other DNA
aaaagaggtggggagtgggtggg

SEQ ID NO: 421 - DNA, 23 bp, synthetic construct
  source 1..23: mol_type = other DNA
tgtgagccagcgcgctggcctgg

SEQ ID NO: 422 - DNA, 23 bp, synthetic construct
  source 1..23: mol_type = other DNA
tgaagaggaggacggggctgagg

SEQ ID NO: 423 - DNA, 23 bp, synthetic construct
  source 1..23: mol_type = other DNA
cggagggctgtgggtggggtagg

SEQ ID NO: 424 - DNA, 23 bp, synthetic construct
  source 1..23: mol_type = other DNA
cacttctagctgtgtggccacgg

SEQ ID NO: 425 - DNA, 23 bp, synthetic construct
  source 1..23: mol_type = other DNA
aaggaaaagaggtggggagtggg

SEQ ID NO: 426 - DNA, 23 bp, synthetic construct
  source 1..23: mol_type = other DNA
ttagagaaggaaaagaggtgggg

SEQ ID NO: 427 - DNA, 23 bp, synthetic construct
  source 1..23: mol_type = other DNA
ggagggctgtgggtggggtaggg

SEQ ID NO: 428 - DNA, 23 bp, synthetic construct
  source 1..23: mol_type = other DNA
aaaagatggggagagatggaagg

SEQ ID NO: 429 - DNA, 23 bp, synthetic construct
  source 1..23: mol_type = other DNA
gagatggaaggaattagagaagg

SEQ ID NO: 430 - DNA, 23 bp, synthetic construct
  source 1..23: mol_type = other DNA
aggaattagagaaggaaaagagg

SEQ ID NO: 431 - DNA, 23 bp, synthetic construct
  source 1..23: mol_type = other DNA
ggataaaacctgaagggatgagg

SEQ ID NO: 432 - DNA, 23 bp, synthetic construct
  source 1..23: mol_type = other DNA
gaaaagaggtggggagtgggtgg

SEQ ID NO: 433 - DNA, 23 bp, synthetic construct
  source 1..23: mol_type = other DNA
tcctcacagtcctgtgaggtagg

SEQ ID NO: 434 - DNA, 23 bp, synthetic construct
  source 1..23: mol_type = other DNA
tctgtaatcacccagcactttgg

SEQ ID NO: 435 - DNA, 23 bp, synthetic construct
  source 1..23: mol_type = other DNA
aaggtatggactgggcacagtgg

SEQ ID NO: 436 - DNA, 23 bp, synthetic construct
  source 1..23: mol_type = other DNA
gaaggaaaagaggtggggagtgg

SEQ ID NO: 437 - DNA, 23 bp, synthetic construct
  source 1..23: mol_type = other DNA
aaaaggagagaaagtagagacgg

SEQ ID NO: 438 - DNA, 23 bp, synthetic construct
  source 1..23: mol_type = other DNA
acagatgtgagccagcgcgctgg

SEQ ID NO: 439 - DNA, 23 bp, synthetic construct
  source 1..23: mol_type = other DNA
ctgtaatcacccagcactttggg

SEQ ID NO: 440 - DNA, 23 bp, synthetic construct
  source 1..23: mol_type = other DNA
cctggggccagggagccaagagg

SEQ ID NO: 441 - DNA, 23 bp, synthetic construct
  source 1..23: mol_type = other DNA
taatcacccagcactttgggagg

SEQ ID NO: 442 - DNA, 23 bp, synthetic construct
  source 1..23: mol_type = other DNA
ctctattatttgtagaaacgggg

SEQ ID NO: 443 - DNA, 23 bp, synthetic construct
  source 1..23: mol_type = other DNA
gaggttcagtcacttgcccgtgg

SEQ ID NO: 444 - DNA, 23 bp, synthetic construct
  source 1..23: mol_type = other DNA
gggaggtggaggctggaggatgg

SEQ ID NO: 445 - DNA, 23 bp, synthetic construct
  source 1..23: mol_type = other DNA
tcacccagcactttgggaggtgg

SEQ ID NO: 446 - DNA, 23 bp, synthetic construct
  source 1..23: mol_type = other DNA
cagctgtcatcccagcactttgg

SEQ ID NO: 447 - DNA, 23 bp, synthetic construct
  source 1..23: mol_type = other DNA
gaggaaatggaggcacagagagg

SEQ ID NO: 448 - DNA, 23 bp, synthetic construct
  source 1..23: mol_type = other DNA
gcctccacctcccaaagtgctgg

SEQ ID NO: 449 - DNA, 23 bp, synthetic construct
  source 1..23: mol_type = other DNA
ggggttttgctttgttgcccagg

SEQ ID NO: 450 - DNA, 23 bp, synthetic construct
  source 1..23: mol_type = other DNA
ctggaggatggcgtgagtccagg

SEQ ID NO: 451 - DNA, 23 bp, synthetic construct
  source 1..23: mol_type = other DNA
cccagcactttgggaggtggagg

SEQ ID NO: 452 - DNA, 23 bp, synthetic construct
  source 1..23: mol_type = other DNA
agctgtcatcccagcactttggg

SEQ ID NO: 453 - DNA, 23 bp, synthetic construct
  source 1..23: mol_type = other DNA
ttttgctttgttgcccaggctgg

SEQ ID NO: 454 - DNA, 23 bp, synthetic construct
  source 1..23: mol_type = other DNA
caggagtttcagaccagcctggg

SEQ ID NO: 455 - DNA, 23 bp, synthetic construct
  source 1..23: mol_type = other DNA
gcaggagagctgcttgagcccgg

SEQ ID NO: 456 - DNA, 23 bp, synthetic construct
  source 1..23: mol_type = other DNA
ccaggagtttcagaccagcctgg

SEQ ID NO: 457 - DNA, 23 bp, synthetic construct
  source 1..23: mol_type = other DNA
tgtcatcccagcactttgggagg

SEQ ID NO: 458 - DNA, 23 bp, synthetic construct
  source 1..23: mol_type = other DNA
gcactttgggaggtggaggctgg

SEQ ID NO: 459 - DNA, 23 bp, synthetic construct
  source 1..23: mol_type = other DNA
ctttgggaggtggaggctggagg

SEQ ID NO: 460 - DNA, 23 bp, synthetic construct
  source 1..23: mol_type = other DNA
gcctcagcctcccaaagtgctgg

SEQ ID NO: 461 - DNA, 23 bp, synthetic construct
  source 1..23: mol_type = other DNA
cctcagcctcccaaagtgctggg

SEQ ID NO: 462 - DNA, 23 bp, synthetic construct
  source 1..23: mol_type = other DNA
gcactttgggaggctgaggcagg

SEQ ID NO: 463 - DNA, 11623 bp, synthetic construct
  source 1..11623: mol_type = other DNA
acttccggtcgggccctcgggtctccccggagcggcggcgcctcctccgcctcctcggcctcctcccggcggagaccccggcgccggtgagtgacggggtgcgtggcccgggggcccgggtgcagcgggggcggggggccccgccctggcccgctgtggtccccagaacgtcccccaacccctagcaagtcagtgcagctcccgccgggcccttgccaagggacccccaatcactgctccccggtaggatcactgcagatagctcccccatgaacgcccccagctaggatggctgccaaagcgctttcattcgtggcgatcccctaaacggctgccagggcccccaaatgatacctcctcgtaggatcccctccacatagcgctccccgtcattgcttcctccaggtcagcctccagatgccatcagcattgcccctccccatgtagggcagctgctcgtgaaccctcttattagtatcctctatataattctccccagagtgtttccttagtgccttgcagcagaactgtcccccgttatctctgtccatacagcaacagcccccaatggccctgaccacctccctccccagcagaacgccccttcgtgggtgtgaaaatactttctattctggtcagcaccaagaatgcctttttcccttctgcaggtcctccagtgattccccttaagaatgcccctttcaaagccacccccccatcgcagcggcacagctccctctagagttccttcacactcacatcctctcccgcctcaggtagaaatatccgcctgcttagctccaggctcccatgacatactcccgtacctcctctcaccccaccctcatcgcggtcagcccgtcttcattacttctgccacagaacagtgtcccgcagtgaggcggtgaagccttccttcccagaatgtgcctcatcctcttcctatggcgtgaacaactgttgccctgacctgcagcttctcacccagctctcaggctatcgtcctggactccctagggaagaccctggacttcactagggtgtgacttcttttctcgtaggcattccttctgcgttgaacgcatattcactattctagctgaagggtataatatacagccacgaagggggtcgatacacacagtgttctcactgtgcggggtctcacagtctagttgatcagacacgagtcgacaaagatacacggggttttgtgggttctctgagagcctgatgaactactttagaggtgaaagctgaattacaagtggcagcttagccatgcaaagatctgggtgagcaggcagcaagaacaacaagtgcaaaggccctgaagtggcggtgagcacggtgagtggaggagcagcaggggaggccggtgagggtggagtggaagatttgaaggcaagaacaagaggaaggaagctttggtctcaaaaggctttggaggtgagggcactgggataaattatctagcctttgtcagctctaccgagcccagaatgttcttcattcattcatacgttcattcgtaaacactggttgaacaactgtttaatgagcacctactttacattgggggtacaacagagaacaaaatagacaagaatccctgtcctcatggagtggatatgctagttgagggggcagatgattaaaagacaaaatacaggccaggcgaggtggctcatgcctgtaatcccagcagttgggaaggccaaagtgggaggatcgctggaggccaggagttcaagaccaacctcggcaacatagtgagaccctgtctttacaggaaaaaaaaaaaaaaaaaaaaaaagctgggtgtggttggtgggcacctgtagtccccagctattcaggaggctgaggtgggagggctgcttgagcccaggagttcgaggctgcagtgaaaaaaaataaaaacaaaaaccaagagtatatgtgcgtgttcagaatatcacgggataaagagatagacctggggggctgtggttcaggatggcctcccccaggattttcctatgtcttgtcatcttgtccctcccaaaagccacacaccaaaaagttatacaagagtaaaaattccccttctcctggacaactgtggcccacttgggggctgctcttaacagcgccctaaccccaggagcgaagtgattgggaggatgagccctggattccactagggttcacagctttgagcaaatcactccctcctttcctagcctcattcattcacttcttcaaacaaaaatgtatcaagccaggaactgttgtaggtcctggggatggaacagtgagcaagagataaaaatccttgccttcaggaagccgatcatctggatggggagaccaacaaaatcaaggcaaagaagtagattatgtgaggttataaatgatgctgagcagtaaggagagaaatgaagcagggttagggagatgaggatgtgggcctgggtgacaggtcattggttactgagaggctcattgagaagacagttgaccaaagatggggaggaggtgaaggggtgagcgagatggttgcctgagagtcagaatattacaggcaacagcaacagccagcgcaaaggccctgcagcaggaatagagtgggcacatgggaagagcagccaggagactgtggaggtcattgtaaggactgtggctttttttttttttttttttgaaaaggagtttcgctcttgttgcccaggctggagtgcagtggtgtgatcttagctcgctgcaacctccgcctcccgggttcaagtgattctcctgcctcagcctcccgagtagctgggattacaggcatgtgccaccacgcccagctaattttgtatttttagtagagatggggtttctccatgttggtcaggctggtctcaaactcccgacctcaggtgatccgcctgccttggcctccaaaaatgctgggattacaggcgtgagccaccgtgcccagtcaggactgtggcttttgaatgagatggggttactgtttggtttgggaccgaggatagacatgctttggttatagtcctcctgtcagtaaaatgggaataataataacacatttgttgtgaagtttgaatgaaacaagccacataaggcacctaagtacaatgcttgttagaggtcaacaaatgttggtgacttgttctggtcttgggtcaagccaagagacttgggatttgttggcagggagatgggtggagcaatgcatgtttccagtatgaagtgggattgggggcgggggtcctccttcattttggagggagaaataccgaaagacagcatgtgatgggactctggagccttgctgcctttcttcaaccactggccctgccactagttaacaatgtgactttagaagtttgtttgtttgtttttgagaaacagagtctcactctgtcacccaggctggagtgcagtggcgtgagctcactgcaacctctgtgtcctgggttcaagcaattatcctgcctcagcttccccactagctggaactacaggtgtgcaccaccacacccggctaatttttttatttttagtagagacggggtttcactatatattggccaggctagtctcgaactcctgactatagttgatccacccacctcggcctcccaaagtgctggcattacaggcatgagccaccatgcctggcctgactttggaagtttaatttatctctctggccctcagtctcttcctttgtatttcagagtcaatgcttgtaaccatcttctagggttactgggaggattcgttgagctcacacacatcagagttacaagagtgctgggctggtagaaagcacacatccagtgttatatgcgcacgttactctttattaacaaaccagaatttcatcacaaggcagcgttgttgcagcagaacggcccacagtctggcatcagaaagacctggaatcagttacctgctttcctctttattggccacatgaccttggaggaatgccttccccatgctgctcctcagtttccccacttataagatgggcccagtcatggtacaggactgtgctgcctccaggcaataacaaatatcaaactggtcaaacaataaatgtgaaccgttaatacacttgctgtgggactcctgtcccttggtggtggtggtggaggggtgggtccctccccatcatgcttctctctgcataaaaccctcctatgacatcccctcttaaaaagataccagtttaggcttgagttgtatgaaagagggaatgtttctggtttcctagtggagattatcataagcctgatcaattctgtccaccactgatgtatccccttcagcatctccacaccctgccctcagtgccctcctcataacatcactgtccccagaattgcttctcccaggaattcctgggatcatttagctccatttctgctacatgtccccctccgttcctctgggagtttgatcagcttagtctcatcgctttgttcattccacaaataccaagatagacattcacagctgtgcatttccctctgagcattgctttggctgcacctcctcagctttggtatgttgtgttttcattttcattaattgctaagcagtttttttgctttttgtttgtttgtgtttttgagatggagtcgcgctcttgttgcccaggctggagtgcagtggcacgatctcagctcactgcaacctccacctcccgagttcaagcgattctcctgcctcagcctcccaagtagctgggattacaggcgcccaccaccaagcctggctaatttttgtatttttagtagagacggggtttcgccatgtaggccagactggtgtcgaactcctaacctcaggtgatcctcctgccttggcctcccaaagtgccgggattacaggtgtgggccaccgcacctggccagcattttctaatgtatgatttctttgtggacccattggtaatttaggaaggtggtgtttccttttcacataccagcagatatttgtcaagcacccacactgggccagtgctacgctaagtgctggggtcacaggggcgagcaagacaggccaagtccttgctttcctagagaggggccaaacagtaaacacacagaaaaggaatgacatgagttggtggcaataactataatgaaacacctgcggctggatgagggactggagagtgatggggtggggagtgctgagtagagacccaaggaggctccagagggagtcttgttgaaatctggcgaagaacactgtaggcagaagagaaagtaaccaccaccctctcccaccaggccccttgtatagacacccctcataacccagaagacatccctaaataaagctcttatcctgatagttctcccctcacctttgttcactcggggaacagcccagtgaaacactgctgtgggggcaagggtgggaatggggttcaagcccattgatccagactcctactccgtgaccatggcaactcactttgcctctccatgccttagttttctaatttatccaacggcgggaacagctgcccctatttagagttgtggggagtcagtgaattaatatgtgtaacgtgcttagaaccgtggcgggcctctaggagatgtccagtcagtgacagctacgattaacctgtgctctagtggagacaagctagtcaggggcttctttccccaaacactcttgccctccctccccatagcgccctaaatcaccccctattccctgcatccttatggcctgtgtttgcctccctggccagtcagccccctccagagctgtctccccaacttaggttccacctacagccttccaattaccaccccagccaagcctgtcctctttccggagatcagattaaacggagggggtgtgaagggcatgggatgttgagtcttgtccaaggacaaggacacccggagctggagactaaaaatatctcactccaggatggcgtttggggtgggggtggaagagacactcacccctcacctgtgggaggagagagcagcccgatggatagaggataaagggtccaggccttggtaggaacccccttgacccccatagcgggctgggcttagagcctggcccccatcgggtagggatgatggacaggaagcgggtcagacctcatgggcccttcaccgactcaccctccgcccacttggtggaggtgcttccccaggccagcaagtagaccaagaactcggagatcagttacgggtgctgagggtctggtgtggtgtggggtacacacatggatacctgccatctggaatgaaaagtcgtgtaaaggaggtgccagagaatgatgccgggggtggagggtcaggagaagccccactaagggcaaggacatgggggggtcaggagaagccccactaagggcaaggatggaactcaagctccctgcttctgttgctaggcttagcccaggtgtgtgatgtcatggatggtacaaatagctcagggggttgaatgcattctgctgatgcatgtgtttcattatgttctgttccaaccctgttgaatctccctgcggtataggggttcccccattttaaacatggagaaagtgtggatcccagtggagatgggatttgaacccaggtctgtccaagcccagggtctgtgttttgattgtatcagaccaccttctgactatatgccagcccccttgctggaatttgtttctcccaggcaagcaaattctcctttctactgtctattaatattaaaagagtgcactccccatactgacttttttcagttctctaggcttgtcctgacttccccatttctactactagtagtaacagtgataacagtatcgtatcttcaattttttaattttagtttttagagccaaggtcttgctctgtcacccaggctggagtacagtgatgtgatcacagctcattgtagccttcacctcccgggctcaagcagctctcctgcctcagcctcccaaagtgctgggatgacagctgtcagccactgtgcccagtccataccttctatgtacggagtgcttacagagtgccaggaaccgccccctaagagccttgcacatatgtgtgtgttgaatcctcacagtcctgtgaggtaggcatgtgctttgtggtccccaggtgtagatgaggaaatggaggcacagagaggttcagtcacttgcccgtggccacacagctagaagtggcagggcttacatttgattctggccccagagtcctccctcctaattacgctgatagaaactgtgattccccttatcattatgctcattttaggataaaacctgaagggatgaggacggagggctgtgggtggggtagggtgcaggagtatggagagacagcagagggtctgagagggtttggaaagaaaactgagtccagaggcccccaggatgcttggacaattggagtctgaaaaggagagaaagtagagacggcccaggccagcgcgctggctcacatctgtaatcacccagcactttgggaggtggaggctggaggatggcgtgagtccaggagtttcagaccagcctgggcaacaaagcaaaaccccgtttctacaaataatagagggcctgcaggtggtgtggaggagagggtcccctctacaaagctgtgtcaggaccccagaaaagatggggagagatggaaggaattagagaaggaaaagaggtggggagtgggtgggagttgagaacacagagagggaaggtgaaggtgagtaggagagtcctggggccagggagccaagaggaagggctgccctttggggtggacaggcagagggactggaagcctaaatctggaaggctagggggtgatccgcacaaggatggatgagtgggcgcacagctccattcacctgcggggagacccagcgccgcggccgtgtcctcagccccgtcctcctcttcacagggtctcccgtctcccacccgccggagatggaggcgaacccagcgggcagcggcgccgggggtggcgggagcagcggcatcgggggcgaggacggggtgcacttccagagctaccccttcgacttcctggaattcctcaaccaccagcgcttcgagcccatggaactgtatggggaacacgccaaggcggtggcggccctgccctgcgcccccggccccccgccgcagcccccgccgcagccccctcccccgcagtatgactacccgccccagtccaccttcaagcccaaggcggaggtgccctcctcgtcctcgtcctcgtcctcctcctcctcctcttcgtcctcctcgtcgtcatcttcgtcctcttcctcttcccaagccaagaagcccgatccgcccctgccgcccgccttcggggcgccccctcctcccctctttgacgctgctttccccactccgcagtggggcatcgtggacctctcggggcaccagcacttgtttgggaacctgaagcgaggagggcccgcgtccgggccgggggtgacgcctgggctgggcgctcccgcgggggccccagggccgcttcctgccccctcgcagaccccgccaggaccccccgcggcggcggcctgcgaccccaccaaggacgacaagggctacttccggagactgaagtacctgatggagcggcgcttcccctgcggcgtgtgccagaagtccttcaagcagtcctcgcacctggtccagcacatgctggtgcactcgggggagaggccctacgaatgcggcgtctgcggccgcacctacaaccacgtgtccagcctcatccgccaccgccgctgccacaaggacgtgccaccggccgcggggggcccgccccagcccggcccccacctcccgccgctgggcctcccagcacccgctgccagcgccgccaccgccgccgccccctccacggtgtcctcgggccctccagccacgcccgtggcgcctgccccctccgcagacgggagcgccgcccctgctggtgttggggtgccccctcctgccaccgggggtggcgatggcccgttcgcctgcccactctgctggaaggttttcaagaagcccagtcacctccaccagcaccagatcatccacacgggcgagaagcccttctcctgctccgtgtgcagcaaaagcttcaaccgcagggagagtctgaagcgccacgtgaagacgcactcggccgacctcctgcgcctgccctgcggcatctgcgggaaggccttccgcgacgcctcctacctcctcaagcaccaggcggcccacgcgggggcgggcgccggggggcctcggcccgtgtacccctgcgacctgtgcggcaagtcctactcggctccgcagagcctgctccgccacaaggccgcccacgccccgcccgctgccgctgcggaggcgcccaaggacggggcggcctcggccccgcagcccccgcccaccttccccccgggcccgtacctcctgccccccgaccctcccaccacagacagcgagaaggcggcggcggccgcggcggcggtggtgtacggcgctgtgcccgtcccgctcctgggcgcccacccgctgctgctcggcggcgcggggaccagcggggcgggaggctcgggcgccagcgtcccaggaaagacgttctgctgcggcatctgcgggcgcggcttcgggcgccgcgagaccctgaagcgccatgagcgcatccacacgggcgagaagccccaccagtgccccgtgtgtgggaagcgcttccgcgaatccttccacttgagcaagcatcacgtggtgcacacgcgcgagcggccctacaagtgcgagctctgcggcaaggtcttcggctacccgcagagcctcacccgccaccgccaggtgcaccggctccagctgccctgcgccctggccggggcagccggcctcccctccacccaaggcacaccgggggcctgtgggcccggggcctcgggcacgtctgcagggcccaccgatgggctgagctacgcctgctcggactgcggcgagcacttcccggatctctttcacgtcatgagtcacaaggaggtccacatggcagagaagccatacggctgcgacgcctgcggcaagaccttcggcttcatcgagaacctcatgtggcacaagctggtccaccaggccgcccccgagcgcctgctcccgcccgcacccggcggcctgcagcccccggacggctccagcggcacggatgcggccagcgtgctggacaacgggctggcgggggaggtgggggcggccgtggcggcactggcaggggtgtctgggggtgaggacgcaggcggggcggcggtggcaggtgctggcgggggtgccagttccggccccgagcgcttcagctgtgccacgtgcggccagagtttcaagcacttcctgggcctcgtgactcacaagtacgtgcacctggtgcgacggaccctgggctgcggcctctgcggccagagcttcgcgggcgcctacgacttgctcctacaccgccgcagccatcggcagaagcggggtttccgctgcccggtgtgcgggaagcgcttctgggaggcggccctgctgatgcgccaccagcgctgccacacggaacagcggccgtaccgatgtggcgtgtgcggccgaggcttcctgcgctcctggtacctgcggcagcaccgcgtggtgcacactggcgagcgggccttcaagtgcggcgtgtgcgccaagcgcttcgcgcagtcgtccagcctggcagagcaccggcggctgcacgctgtggcccggccccagcgctgcagcgcctgtggcaagaccttccgctaccgctccaacctgctggagcaccagcggctgcacctgggcgagcgcgcctaccgctgtgagcactgcggcaagggcttcttctacctgagctccgtgctgcgccaccagcgcgcccatgagccgccgcggcccgagctccgctgccccgcctgcctcaaggccttcaaggatcccggctacttccgtaagcacctggctgcccaccagggcggccggcccttccgctgctcctcctgcggcgagggcttcgccaacacctacggcctcaagaaacaccgcctggcgcacaaggccgagaacctcggggggcctggagcaggggcgggcaccttggccgggaaggatgcctgaccgaggggttcccatcccactcccatcaaaagcccccttctggactcccacctcccaggactgatcagactcttcccccctcctcgctgttgccccatccttcagaacttcacacggactggcgaccttcagggcgcacgcccgacaggctcaagactgaatcactcccatcctcgacctctctgccctcccctcatcccatcagacactgaaccctatcctccgtccaaccctcgtttgtgacccgcatcagcccccgccccagcagcactctgcccccagtaagttttggcggagatgggtctgaaccgcccctccccctcctttggaatctggctggacagtggagtatgagcagagttgggagggcacaagggagtgctgggtgctttttggggtgggggggtggggggcggggtggcagacgcggcttgtacagagcggagaataataaatcttaccatgagggcc

(19) United States
(12) Patent Application Publication
(10) Pub. No.: US 2025/0099613 A1
(43) Pub. Date: Mar. 27, 2025

(54) Compositions And Methods For Regulating ZNF865

(71) Applicant: University of Utah Research Foundation, Salt Lake City, UT (US)

(72) Inventors: Robert Bowles, Salt Lake City, UT (US); Hunter Levis, Salt Lake City, UT (US); Jacob Weston, Salt Lake City, UT (US)

(21) Appl. No.: 18/785,710

(22) Filed: Jul. 26, 2024

Related U.S. Application Data
(60) Provisional application No. 63/515,733, filed on Jul. 26, 2023.

(51) Int. Cl. A61K 48/00; A61K 38/17; C12N 9/22; C12N 15/11; C12N 15/86

(52) U.S. Cl. CPC A61K 48/005; A61K 38/1709; C12N 9/22; C12N 15/111; C12N 15/86; C07K 2319/71; C12N 2310/20; C12N 2740/15043

(57) ABSTRACT

Disclosed are polypeptides comprising two heterologous polypeptide domains, wherein the first polypeptide domain comprises a Cas protein and wherein the second polypeptide domain comprises a zinc finger protein 865 (ZNF865) or fragment thereof. Disclosed are polynucleotides capable of encoding a polypeptide comprising two heterologous polypeptide domains, wherein the first polypeptide domain comprises a Cas protein and wherein the second polypeptide domain comprises ZNF865 or fragment thereof. Disclosed are vectors comprising one or more of the polynucleotides. Disclosed are methods of using the disclosed polypeptides, polynucleotides and vectors.

### CROSS-REFERENCE TO RELATED APPLICATIONS

This application claims the benefit of U.S. Provisional Patent Application No. 63/515,733, filed Jul. 26, 2023, which is incorporated by reference herein in its entirety.

### STATEMENT REGARDING FEDERALLY SPONSORED RESEARCH

This invention was made with government support under Grant Number R01 AR074998 awarded by the National Institutes of Health. The government has certain rights in this invention.

### REFERENCE TO SEQUENCE LISTING

The Sequence Listing submitted Dec. 9, 2024 as a text file named “21101.0444U2.xml,” created on Dec. 9, 2024, and having a size of 430,614 bytes is hereby incorporated by reference pursuant to 37 C.F.R. § 1.52(e)(5).

### BACKGROUND

Degenerative disc disease (DDD) is the leading cause of disability worldwide, characterized by the breakdown of intervertebral discs (IVD). Stem cell therapies using adipose-derived stem cells (ASCs) are promising options to treat DDD because of stem cells' ability to regenerate and restore functional tissue to the IVD. Recently, CRISPR-guided activation (CRISPRa) genome-wide perturbation screens have been used to profile gene function.

Currently, CRISPR-activation systems are used to upregulate target gene expression. The dCas9-VPR, dCas9-VP64, Synergistic Activation Mediator (SAM), and SunTag are the four primary systems used for targeted gene upregulation. The dCas9-VP64 is generation 1 CRISPRa which showed modest degrees, −2-fold increases in activation levels by using VP64 which recruits transcriptional machinery to the targeted gene. The dCas9-VPR system uses a tripartite complex of VP64-p65-Rta, where p65 and Rta are transcriptional activators that help recruit additional transcription factors to the target gene. SAM uses an engineered gRNA that increases transcription. The gRNA is engineered specific for the system and creates a dCas9-VP64 fusion protein that recruits activation domains for gene upregulation. SunTag uses a repeating peptide array that contains multiple copies of VP64, which allows for the recruitment of transcriptional machinery for targeted gene upregulation. There are two newer methods for targeted gene upregulation using the CRISPR system, dCas9-CBP and SPH. Both supposedly increase target gene expression 2 to 3-fold higher compared to SAM, SunTag, and VPR. These two new methods have yet to be rigorously compared, but initial data shows improvements. Additionally, dCas9 has been fused to the P300 protein for targeted gene activation. This system showed a method to fuse a protein for targeted gene upregulation. This system has had inconsistent results, but a similar method could be used with the ZNF865 gene/protein described herein.

### BRIEF SUMMARY

Disclosed are polypeptides comprising two heterologous polypeptide domains, wherein the first polypeptide domain comprises a Cas protein and wherein the second polypeptide domain comprises a zinc finger protein 865 (ZNF865) or fragment thereof.

Disclosed are polynucleotides capable of encoding a polypeptide comprising two heterologous polypeptide domains, wherein the first polypeptide domain comprises a Cas protein and wherein the second polypeptide domain comprises ZNF865 or fragment thereof. In some aspects, the polynucleotides can be referred to as nucleic acid constructs.

Disclosed are vectors comprising a nucleic acid sequence that encodes ZNF865.

Disclosed are vectors comprising a polynucleotide capable of encoding a polypeptide comprising two heterologous polypeptide domains, wherein the first polypeptide domain comprises a Cas protein and wherein the second polypeptide domain comprises ZNF865 or fragment thereof.

Disclosed are vectors comprising a nucleic acid sequence of a ZNF865-specific gRNA.

Disclosed are compositions comprising the disclosed fusion proteins, nucleic acid constructs or vectors.

Disclosed are recombinant cells comprising one or more of the disclosed nucleic acid constructs or vectors.

Disclosed are CRISPR-Cas systems comprising one or more vectors comprising one or more nucleotide sequences encoding a CRISPR-Cas system guide RNA (gRNA), wherein the gRNA hybridizes with a target sequence of ZNF865 in a cell; and a nucleotide sequence encoding a deactivated Cas (dCas) protein fused to a transcriptional activator, wherein the nucleotide sequences for the gRNA and the deactivated Cas protein fused to a transcriptional activator are located on the same or different vectors of the same system, wherein the gRNA targets and hybridizes with the target sequence and directs the deactivated Cas protein fused to a transcriptional activator to the ZNF865.

Disclosed are CRISPR-Cas systems comprising one or more vectors comprising one or more nucleotide sequences encoding a CRISPR-Cas system guide RNA (gRNA), wherein the gRNA hybridizes with a target sequence of a DNA locus in a cell; and a nucleotide sequence encoding a deactivated Cas protein fused to ZNF865, wherein the nucleotides encoding the gRNA and the dCas protein fused to ZNF865 are located on the same or different vectors of the same system, wherein the gRNA targets and hybridizes with the target sequence and directs the deactivated Cas protein fused to ZNF865 to the DNA locus.

Disclosed are methods of increasing ZNF865 in a cell comprising contacting a cell with a vector comprising a nucleic acid sequence encoding a ZNF865 protein (gene therapy); a recombinant ZNF865 protein or fragment thereof (protein therapy); or one or more vectors comprising one or more nucleic acid sequences encoding a ZNF865-specific guide RNA (gRNA) and a dCas9 fused to a transcriptional activator, wherein the transcriptional activator upregulates ZNF865 (CRISPR therapy).

Disclosed are methods of upregulating expression of a target gene comprising contacting a cell with a therapeutically effective amount of a vector comprising a nucleic acid sequence encoding a ZNF865 protein; a therapeutically effective amount of a ZNF865 protein; or one or more vectors comprising one or more nucleic acid sequences encoding a target gene-specific guide RNA (gRNA) and a dCas9 fused to ZNF865, wherein ZNF865 upregulates the target gene.

Disclosed are methods of increasing deposition of aggrecan and/or collagen II in the extracellular matrix of a cell comprising administering to a cell a therapeutically effective amount of a vector comprising a nucleic acid sequence encoding a ZNF865 protein; a therapeutically effective amount of a ZNF865 protein; one or more vectors comprising one or more nucleic acid sequences encoding a ZNF865 specific guide RNA (gRNA) and a dCas9 fused to a transcriptional activator, wherein the transcriptional activator upregulates ZNF865; or one or more vectors comprising one or more nucleic acid sequences encoding a target gene-specific guide RNA (gRNA) and a dCas9 fused to ZNF865, wherein the target gene is aggrecan and/or collagen II, wherein ZNF865 increases deposition of aggrecan and/or collagen II in the extracellular matrix of the cell.

Disclosed are methods of treating degenerative disc disease comprising administering to a subject a therapeutically effective amount of a vector comprising a nucleic acid sequence encoding a ZNF865 protein; a therapeutically effective amount of a ZNF865 protein; a ZNF865 specific gRNA and a dCas9 fused to a transcriptional activator, wherein the transcriptional activator upregulates ZNF865; or one or more vectors comprising one or more nucleic acid sequences encoding a target gene-specific guide RNA (gRNA) and a dCas9 fused to ZNF865, wherein the target gene is aggrecan and/or collagen II.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A-1F show an example of pellet culture analysis showing ZNF865 amplifies ECM deposition of VPR-ACAN/Col2 upregulated ASCs. (FIG. 1A) Gross pellet morphology shows increased tissue deposition in ZNF865-edited cells compared to the NTC (n=10, scale bar is 1 mm) and (FIG. 1B) representative alcian blue staining shows increased sGAG deposition in ZNF865-edited cells compared to the NTC (n=5, scale bar is 100 μm). (FIG. 1C) ZNF865-edited cells show increased collagen deposition per pellet and (FIG. 1D) increased retention of collagen within the pellet compared to the NTC (*=p<0.05, n=10). (FIG. 1E) ZNF865-edited cells show increase sGAG deposition per pellet and (F) increased retention of sGAGs within the pellet compared to the NTC (*=p<0.05, n=10). NTC is nontarget control.

FIGS. 2A-2D show additional analysis of ACAN/Col2-ZNF865 edited pellets (FIG. 2A) verifies ZNF865 upregulation with qRT-PCR (n=5), FIG. 2(B) shows significant increases in pellet volume compared to the NTC and shows significant increases in (FIG. 2C) total collagen produced and (FIG. 2D) total sGAG produced by ZNF865-edited cells compared to ACAN/Col2-NTC edited cells (*=p<0.05, n=10). NTC is nontarget control.

FIGS. 3A-3C show an example RNA sequencing analysis showing significant changes in differential gene expression for three different cell types. (FIG. 3A) Naïve HEK293T cells upregulated with ZNF865 show 3,482 significantly differentially expressed genes compared to a NTC, and (FIG. 3B) naïve ASCs upregulated with ZNF865 show 7,409 significantly differentially expressed genes compared to a NTC. (FIG. 3C) VPR-ACAN/Col2 ASCs upregulated with ZNF865 show 8,937 genes that are significantly differentially expressed genes compared to ACAN/Col2-NTC cell line. P<0.05, NTC is nontarget control.

FIGS. 4A-4E show an example flow cytometry cell cycle analysis showing a shift in cell cycle in ZNF865-edited cells. The first peak represents cells in the G0/G1 phase, the flat section between the two peaks represents the S-phase, and the second peak corresponds to the G2/M phase of the cell cycle. Representative flow cytometry histograms for (FIG. 4A) HEK293T-NTC, (FIG. 4B) HEK293T-ZNF865, (FIG. 4C) ASCs-NTC, and (FIG. 4D) ASCs-ZNF865 shows a shift in number of cells in each stage of the cell cycle. (FIG. 4E) Quantified percentages of cells within each stage of the cell cycle. NTC is nontarget control.

FIGS. 5A-5D show CRISPR-guided gene modulation of ZNF865 affects proliferation of rate of HEK293T cells and ASCs. Upregulation of ZNF865 in (FIG. 5A) HEK293T cells and (FIG. 5B) ASCs shows increased proliferation rates compared to the NTC over 5 days of culture (*=p<0.05, n=4). Downregulation of ZNF865 using the KRAB repressor domain in (FIG. 5C) HEK293T cells and (FIG. 5D) ASCs shows cell death within days of ZNF865 suppression (*=p<0.05, #=p<0.001, n=4). NTC is nontarget control.

FIGS. 6A-6H show RNA-seq on CRISPRa ZNF865 upregulated cells shows thousands of differentially expressed genes. Representative plots showing the thousands of significantly differentially expressed genes due to upregulation of ZNF865 in (FIG. 6A) Naïve HEK cells, (FIG. 6B) Naïve ASCs, and (FIG. 6C) ACAN/Col2 ASCs. Gene Ontology analysis shows the top molecular processes affected by ZNF865 upregulation in (FIG. 6D) Naïve HEK 293 cells, (FIG. 6E) Naïve ASCs, and (FIG. 6F) ACAN/Col2 ASCs. The top 8 overlapping molecular processes affected by ZNF865 upregulation displays Cell Cycle, Cellular Senescence, DNA Replication, Protein Processing in the ER, RNA Transport, Mismatch Repair, Base Excision Repair, and Autophagy. (FIG. 6G) Network analysis shows the top transcription factor (TF)-Gene Interactions occurring due to upregulation of ZNF865 [4,5]. (FIG. 6H) Overall, thousands of interactions are occurring with the top TF's being listed in alphabetical order. NTC is nontarget control.

FIGS. 7A-7G show targeted upregulation of ZNF865 drives entry into the cell cycle in HEK 293 cells and ASCs. (FIG. 7A) The CRISPRa system utilizing the VPR effector molecule acts like a synthetic transcription factor, recruiting RNA polymerase II and effectively upregulating target gene expression. (FIG. 7B) The dCas9-VPR expression cassette and targeted gRNA expression cassette for ZNF865 or NTC upregulation. (FIG. 7C) Representative plots showing the gating strategy to select for cell cycle analysis. (FIG. 7D) qRT-PCR verifies upregulation of ZNF865 expression by almost 8-fold compared to baseline expression. (FIG. 7E) Increased ZNF865 expression shifts the percentage of cells in each stage of the cell cycle, with cells entering the cell cycle at a higher rate in the ZNF865-edited cells compared to the -NTC cells. Additionally, there is an increase in average doubling rates of both cell types edited with ZNF865 compared to the NTC. Representative histograms showing the shift in cell cycle between the -NTC and -ZNF865 edited as well as increased proliferation rates in (FIG. 7F) HEK 293 cells and (FIG. 7G) ASCs. (*=p<0.05, n=4). NTC is nontarget control.

FIGS. 8A-8F show targeted downregulation of ZNF865 using CRISPRi induces cell death in HEK 293 cells and ASCs. (FIG. 8A) The CRISPRi system utilizes the effector molecule KRAB which tri-methylates the histone, preventing transcription and suppressing targeted gene expression. (FIG. 8B) qRT-PCR verifies downregulation of ZNF865, with guides 1, 4, 2, and 3 significantly suppressing ZNF865 expression, percent expression is displayed on the plot. (FIG. 8C) Downregulation of ZNF865 leads to prevention of cells entering the cell cycle with a buildup of cells in the G0/G1 phase. Downregulation of ZNF865 in (FIG. 8D) HEK 293 cells and (FIG. 8E) ASCs induces to cell death within 3 or 4 days, respectively (*=p<0.05, #=p<0.001, n=4). (FIG. 8F) Representative images showing ASC morphology on Day 2 (scale bar=200 μm). NTC is nontarget.

FIGS. 9A-9F show downregulation of ZNF865 in primary hNPCs indicates ZNF865 is a regulator of cellular senescence. (FIG. 9A) Cell proliferation of hNPCs after transduction with KRAB-ZNF865 Guide 2 or Guide 3 compared to the KRAB-NTC shows no cell proliferation for Guide 2 or 3. (FIG. 9B) Representative β-gal staining shows increased staining for Guide 3 after 1 and 3-weeks of culture, showing nearly 80% of cells stained positively for β-gal staining at 3-weeks. (FIG. 9C) Quantitative β-gal staining shows significant increases in staining at 1 and 3-weeks of culture for Guide 2 and Guide 3 compared to the control, with Guide 3 showing nearly 80% positively stained cells (*=p<0.05, n=4). (FIG. 9D) Representative immunofluorescence images showing increased p16 staining for KRAB-ZNF865 Guide 3 3-weeks after transduction. Quantified (FIG. 9E) p16 staining and (FIG. 9F) p21 shows a significant increase in positively stained cells 3-weeks after transduction (*=p<0.05, n=4).

FIGS. 10A-10L show targeted ZNF865 upregulation amplifies cell phenotype in ACAN/Col2 ASCs and Jurkat Cells. (FIG. 10A) The CRISPRa multiplex upregulation expression cassettes for the upregulation system and targeted genes Col2A1, ACAN, and ZNF865. ZNF865 upregulation qualitatively shows increased (FIG. 10B) tissue deposition (scale bar=1 mm) and (FIG. 10C) sGAG deposition (scale bar=100 μm). When expanding ACAN/Col2-edited cells in monolayer, (FIG. 10D) ZNF865-edited cells proliferate significantly faster compared to the ACAN/Col2-NTC. Biochemical analysis shows significant increases (FIG. 10E) concentration of sGAG per pellet and FIG. 10(F) retention of sGAG within the ZNF865-edited pellets. There are also significant increases in (FIG. 10G) collagen per pellet and (FIG. 10H) retention of collagen per pellet in ZNF865-edited pellets. There is no significant difference in (FIG. 10I) DNA content per pellet between the two groups. In Jurkat-edited cells we see a similar significant increase in (FIG. 10J) cell proliferation, in addition significant increases in (FIG. 10K) IL-2 and (FIG. 10L) IFN-y secretion was seen after stimulation with Con A due to ZNF865 upregulation indicating enhanced protein processing and cytokine production due to -ZNF865 upregulation compared to the NTC (*=p<0.05, n=4-10). NTC is nontarget control.

FIGS. 11A-11I show multiplex upregulation of ZNF865 with ACAN/Col2 shows dramatic increases in cartilage deposition in engineered disc without the use of growth factors. (FIG. 11A) Representative schematic of the CRISPRa system and expression cassettes showing increased expression and targeted upregulation of ACAN, Col2A1, and ZNF865. (FIG. 11B) Workflow schematic of cell seeding and culture of DAPS over 5-weeks of total culture. (FIG. 11C) Representative Alcian blue/picrosirius red dual stains showing dramatic increases in ECM deposition between the naïve ASCs, ACAN/Col2 upregulation, and ACAN/Col2-ZNF865 upregulation. (FIG. 11D). Over the course of 5-weeks only 1 successful DAPS was formed with the naïve ASCs, 3 with the ACAN/Col2 ASCs, and 9 with the ACAN/Col2-ZNF865 ASCs. (FIG. 11E) Representative picrosirius red staining and (FIG. 11F) Alcian blue staining showing the NP and AF regions of the engineered disc showing dramatically more cartilage and sGAG deposition in the ACAN/Col2-ZNF865 ASCs compared to the Naïve and ACAN/Col2 ASCs. (FIG. 11G) Representative counts for ACAN, Col2A1, and Col10A1 in our ZNF865-edited cells verifying the overall cell phenotype is maintained. (FIG. 11H) ZNF865 upregulation significantly increases expression of IL1R1a and TNFR2 and decreases TNFR1, IL1R1, and IL6 expression. (FIG. 11I) Upregulation of ZNF865 decreases expression of key SASP genes (*=p<0.05). NTC is Nontarget Control.

FIGS. 12A-12C show a ZNF865 gene layout (FIG. 12A), with one intron and two exons. (FIG. 11B) The bioinformatically predicted structure of ZNF865 showing the disordered regions in grey, 20 different zinc finger (ZNF) domains in pink, 2 transactivation domains (TADs) in blue, and 2 TGEKP cross-linking domains in orange. (FIG. 11C) ZNF865 is broadly expressed across all cell types.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Headings are provided for convenience only and are not to be construed to limit the invention in any manner. Embodiments illustrated under any heading or in any portion of the disclosure may be combined with embodiments illustrated under the same or any other heading or other portion of the disclosure.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a polypeptide” includes a plurality of such polypeptides, reference to “the vector” is a reference to one or more vectors and equivalents thereof known to those skilled in the art, and so forth.

The expression “operationally linked” or “operably linked” means that the promoter sequence is positioned relative to the coding sequence of the gene of interest such that transcription is able to start. This means that the promoter is positioned upstream of the coding sequence, at a distance enabling the expression of the coding sequence.

The term “percent (%) homology” is used interchangeably herein with the term “percent (%) identity” and refers to the level of nucleic acid or amino acid sequence identity when aligned with a wild type sequence using a sequence alignment program. For example, as used herein, 80% homology means the same thing as 80% sequence identity determined by a defined algorithm, and accordingly a homologue of a given sequence has greater than 80% sequence identity over a length of the given sequence. Exemplary levels of sequence identity include, but are not limited to, 80, 85, 90, 95, 98% or more sequence identity to a given sequence, e.g., the coding sequence for anyone of the inventive polypeptides, as described herein. Exemplary computer programs which can be used to determine identity between two sequences include, but are not limited to, the suite of BLAST programs, e.g., BLASTN, BLASTX, and TBLASTX, BLASTP and TBLASTN, publicly available on the Internet. See also, Altschul, et al., 1990 and Altschul, et al., 1997. Sequence searches are typically carried out using the BLASTN program when evaluating a given nucleic acid sequence relative to nucleic acid sequences in the GenBank DNA Sequences and other public databases. The BLASTX program is preferred for searching nucleic acid sequences that have been translated in all reading frames against amino acid sequences in the GenBank Protein Sequences and other public databases. Both BLASTN and BLASTX are run using default parameters of an open gap penalty of 11.0, and an extended gap penalty of 1.0, and utilize the BLOSUM-62matrix. (See, e.g., Altschul, S. F., et al., Nucleic Acids Res. 25:3389-3402, 1997.) A preferred alignment of selected sequences in order to determine “% identity” between two or more sequences, is performed using for example, the CLUSTAL-W program in Mac Vector version 13.0.7, operated with default parameters, including an open gap penalty of 10.0, an extended gap penalty of 0.1, and a BLOSUM 30 similarity matrix.

As used herein, the term “wild-type” refers to a gene or gene product which has the characteristics of that gene or gene product when isolated from a naturally-occurring source.

The terms “variant” and “mutant” are used interchangeably herein. As used herein, the term “mutant” refers to a modified nucleic acid or protein which displays the same characteristics when compared to a reference nucleic acid or protein sequence. A variant can be at least 65, 70, 75, 80, 85, 90, 95, or 99 percent homologues to a reference sequence. In some aspects, a reference sequence can be SEQ ID NO: 1 or SEQ ID NO: 421. Variants can also include nucleotide sequences that are substantially similar to sequences of miRNA disclosed herein. A “variant” can mean a difference in some way from the reference sequence other than just a simple deletion of an N- and/or C-terminal nucleotide. Variants can also or alternatively include at least one substitution and/or at least one addition, there may also be at least one deletion. Alternatively or in addition, variants can comprise modifications, such as non-natural residues at one or more positions with respect to a reference nucleic acid or protein.

Substitutions, deletions, insertions or any combination thereof may be used to arrive at a final derivative or variant. Generally, these changes are done on a few nucleotides to minimize the alteration of the molecule. However, larger changes may be tolerated in certain circumstances.

Generally, the nucleotide identity between individual variant sequences can be at least 90%, 91%, 92%, 93%, 94%, 95%, 96%, 97%, 98%, 99%, or 100%. Thus, a “variant sequence” can be one with the specified identity to the parent or reference sequence (e.g. wild-type sequence) of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence. For example, a “variant sequence” can be a sequence that contains 1, 2, or 3 4 nucleotide base changes as compared to the parent or reference sequence of the invention, and shares or improves biological function, specificity and/or activity of the parent sequence.

Thus, a “variant sequence” can be one with the specified identity to the parent sequence of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence. The variant sequence can also share at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of a reference sequence (e.g. wild-type sequence, SEQ ID NO:1 or SEQ ID NO: 421).

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof

By an “effective amount” of a composition as provided herein is meant as a sufficient amount of the composition to provide the desired effect. The exact amount required will vary from subject to subject, depending on the species, age, and general condition of the subject, the severity of disease (or underlying genetic defect) that is being treated, the particular composition used, its mode of administration, and the like. Thus, it is not possible to specify an exact “effective amount.” However, an appropriate “effective amount” may be determined by one of ordinary skill in the art using only routine experimentation.

By “treat” is meant to administer a peptide, nucleic acid, vector, or composition of the invention to a subject, such as a human or other mammal (for example, an animal model), that has an increased susceptibility for developing degenerative disc disease, or that has degenerative disc disease, in order to prevent or delay a worsening of the effects of the disease or condition, or to partially or fully reverse the effects of the disease.

By “prevent” is meant to minimize the chance that a subject who has an increased susceptibility for developing degenerative disc disease will end up with degenerative disc disease.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Cas9 and ZNF865 Fusion Protein

In some aspects, ZNF865 can act to boost transcriptional activity of a target gene in CRISPR activation methods. Thus, disclosed herein are Cas-ZNF865 fusion proteins.

Disclosed are polypeptides comprising two heterologous polypeptide domains, wherein the first polypeptide domain comprises a Cas protein and wherein the second polypeptide domain comprises a zinc finger protein 865 (ZNF865) or fragment thereof.

In some aspects, ZNF865 can be a full length ZNF865 or can be a fragment thereof. In some aspects, the full length ZNF865 comprises the amino acid sequence MEANPAGSGAGGGGSSGIGGEDGVHFQSYPFDFLEFLNHQRFEPMELYGEHAKAVAAL PCAPGPPPQPPPQPPPPQYDYPPQSTFKPKAEVPSSSSSSSSSSSSSSSSSSSSSSSSSQAKKP DPPLPPAFGAPPPPLFDAAFPTPQWGIVDLSGHQHLFGNLKRGGPASGPGVTPGLGAPA GAPGPLPAPSQTPPGPPAAAACDPTKDDKGYFRRLKYLMERRFPCGVCQKSFKQSSHLV QHMLVHSGERPYECGVCGRTYNHVSSLIRHRRCHKDVPPAAGGPPQPGPHLPPLGLPAP AASAATAAAPSTVSSGPPATPVAPAPSADGSAAPAGVGVPPPATGGGDGPFACPLCWK VFKKPSHLHQHQIIHTGEKPFSCSVCSKSFNRRESLKRHVKTHSADLLRLPCGICGKAFR DASYLLKHQAAHAGAGAGGPRPVYPCDLCGKSYSAPQSLLRHKAAHAPPAAAAEAPK DGAASAPQPPPTFPPGPYLLPPDPPTTDSEKAAAAAAAVVYGAVPVPLLGAHPLLLGGA GTSGAGGSGASVPGKTFCCGICGRGFGRRETLKRHERIHTGEKPHQCPVCGKRFRESFH LSKHHVVHTRERPYKCELCGKVFGYPQSLTRHRQVHRLQLPCALAGAAGLPSTQGTPG ACGPGASGTSAGPTDGLSYACSDCGEHFPDLFHVMSHKEVHMAEKPYGCDACGKTFG FIENLMWHKLVHQAAPERLLPPAPGGLQPPDGSSGTDAASVLDNGLAGEVGAAVAAL AGVSGGEDAGGAAVAGAGGGASSGPERFSCATCGQSFKHFLGLVTHKYVHLVRRTLG CGLCGQSFAGAYDLLLHRRSHRQKRGFRCPVCGKRFWEAALLMRHQRCHTEQRPYRC GVCGRGFLRSWYLRQHRVVHTGERAFKCGVCAKRFAQSSSLAEHRRLHAVARPQRCS ACGKTFRYRSNLLEHQRLHLGERAYRCEHCGKGFFYLSSVLRHQRAHEPPRPELRCPAC LKAFKDPGYFRKHLAAHQGGRPFRCSSCGEGFANTYGLKKHRLAHKAENLGGPGAGA GTLAGKDA (SEQ ID NO: 1). In some aspects, a fragment of ZNF865 is an amino acid sequence shorter than SEQ ID NO:1 that retains its transcription regulatory activity (e.g. ability to activate a gene of interest, such as Aggrecan or Collagen II). In some aspects, a fragment of ZNF65 can comprise or consist of amino acids 1-769 of SEQ ID NO: 1. In some aspects, a fragment of ZNF65 can comprise or consist of the amino acid sequence: MEANPAGSGAGGGGSSGIGGEDGVHFQSYPFDFLEFLNHQRFEPMELYGEHAKAVAAL PCAPGPPPQPPPQPPPPQYDYPPQSTFKPKAEVPSSSSSSSSSSSSSSSSSSSSSSSSSQAKKP DPPLPPAFGAPPPPLFDAAFPTPQWGIVDLSGHQHLFGNLKRGGPASGPGVTPGLGAPA GAPGPLPAPSQTPPGPPAAAACDPTKDDKGYFRRLKYLMERRFPCGVCQKSFKQSSHLV QHMLVHSGERPYECGVCGRTYNHVSSLIRHRRCHKDVPPAAGGPPQPGPHLPPLGLPAP AASAATAAAPSTVSSGPPATPVAPAPSADGSAAPAGVGVPPPATGGGDGPFACPLCWK VFKKPSHLHQHQIIHTGEKPFSCSVCSKSFNRRESLKRHVKTHSADLLRLPCGICGKAFR DASYLLKHQAAHAGAGAGGPRPVYPCDLCGKSYSAPQSLLRHKAAHAPPAAAAEAPK DGAASAPQPPPTFPPGPYLLPPDPPTTDSEKAAAAAAAVVYGAVPVPLLGAHPLLLGGA GTSGAGGSGASVPGKTFCCGICGRGFGRRETLKRHERIHTGEKPHQCPVCGKRFRESFH LSKHHVVHTRERPYKCELCGKVFGYPQSLTRHRQVHRLQLPCALAGAAGLPSTQGTPG ACGPGASGTSAGPTDGLSYACSDCGEHFPDLFHVMSHKEVHMAEKPYGCDACGKTFG FIENLMWHKLVHQAAPERLLPPAPGGLQPPDGSSGTDAASVLDNGLAGEVGAAVAAL AGVSG (SEQ ID NO:2). In some aspects, a fragment of ZNF65 can comprise or consist of amino acids 770-1059 of SEQ ID NO:1. In some aspects, a fragment of ZNF65 can comprise or consist of the amino acid sequence:

(SEQ ID NO: 3)
GEDAGGAAVAGAGGGASSGPERFSCATCGQSFKHFLGLVTHKYVHLVRR
TLGCGLCGQSFAGAYDLLLHRRSHRQKRGFRCPVCGKRFWEAALLMRHQ
RCHTEQRPYRCGVCGRGFLRSWYLRQHRVVHTGERAFKCGVCAKRFAQS
SSLAEHRRLHAVARPQRCSACGKTFRYRSNLLEHQRLHLGERAYRCEHC
GKGFFYLSSVLRHQRAHEPPRPELRCPACLKAFKDPGYFRKHLAAHQGG
RPFRCSSCGEGFANTYGLKKHRLAHKAENLGGPGAGAGTLAGKDA.

In some aspects, the ZNF865 protein can be a variant of any of the ZNF865 proteins described herein. For example, the ZNF865 protein can comprise an amino acid sequence that is at least 60, 65, 70, 75, 80, 85, 90, 95, or 99% identical to SEQ ID NOs:1, 2 or 3.

In some aspects, the Cas protein can be Cas9. In some aspects, the Cas protein can be Cas9 from Streptococcus pyogenes, Streptococcus thermophiles, or Treponema Centicola. In some aspects, the Cas protein is a dCas protein. Thus, in some aspects, the Cas9 can be dCas9. In some aspects, the Cas protein can be any variant of dCas that does not have nucleolytic activity. In some aspects, the Cas protein can be codon optimized for expression in the cell. In some aspects the dCas9 protein comprises the amino acid sequence of:

(SEQ ID NO: 4)
MDKKYSIGLAIGTNSVGWAVITDEYKVPSKKFKVLGNTDRHSIKKNLIG
ALLFDSGETAEATRLKRTARRRYTRRKNRICYLQEIFSNEMAKVDDSFF
HRLEESFLVEEDKKHERHPIFGNIVDEVAYHEKYPTIYHLRKKLVDSTD
KADLRLIYLALAHMIKFRGHFLIEGDLNPDNSDVDKLFIQLVQTYNQLF
EENPINASGVDAKAILSARLSKSRRLENLIAQLPGEKKNGLFGNLIALS
LGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQL
PEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIE
KILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFN
ASLGTYHDLLKIIKDKDFLDNEENEDILEDIVLTLTLFEDREMIEERLK
TYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKK
GILQTVKVVDELVKVMGRHKPENIVIEMARENQTTQKGQKNSRERMKRI
EEGIKELGSQILKEHPVENTQLQNEKLYLYYLQNGRDMYVDQELDINRL
SDYDVDAIVPQSFLKDDSIDNKVLTRSDKNRGKSDNVPSEEVVKKMKNY
WRQLLNAKLITQRKFDNLTKAERGGLSELDKAGFIKRQLVETRQITKHV
AQILDSRMNTKYDENDKLIREVKVITLKSKLVSDFRKDFQFYKVREINN
YHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGR
DFATVRKVLSMPQVNIVKKTEVQTGGFSKESILPKRNSDKLIARKKDWD
PKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAA
FKYFDTTIDRKRYTSTKEVLDATLIHQSITGLYETRIDLSQLGGD.

In some aspects, the Cas protein can be a variant of any of the Cas proteins described herein or any known Cas proteins. For example, the Cas protein can be at least 60, 65, 70, 75, 80, 85, 90, 95, or 99% identical to SEQ ID NO:4.

In some aspects, the ZNF865 is present on the N-terminal or C-terminal end of the Cas protein. Thus, in some aspects, the polypeptide can comprise the order of Cas protein-ZNF865 or ZNF865-Cas protein.

In some aspects, the disclosed polypeptides comprising two heterologous polypeptide domains, wherein the first polypeptide domain comprises a Cas protein and wherein the second polypeptide domain comprises a zinc finger protein 865 (ZNF865) or fragment thereof, and can further comprise a tag. In some aspects, the tag can be used for purification (purification tag) and/or for labeling (labeling tag). In some aspects, a purification tag can be, but is not limited to, FLAG, histidines (e.g. 6XHis), glutathione S-transferase, maltose binding protein, biotin, hemagglutinin, or c-myc. In some aspects, a labeling tag can be any tag used to label, locate or identify the disclosed polypeptides. In some aspects, a labeling tag can be, but is not limited to, FLAG, fluorescent dye, isotope, biotin, or beta-galatosidase. In some aspects, the purification tag and labeling tag can be the same. In some aspects, the tag can be at the N-terminal or C-terminal end of the disclosed polypeptides.

In some aspects, the disclosed polypeptides can further comprise a linker. For example, a linker can be between the disclosed polypeptides and a tag. In some aspects, the linker can be at the N-terminal or C-terminal end of the disclosed polypeptides. In some aspects, the linker can be a polypeptide linker. In some aspects, the linker can be a short, flexible fragment that can be about 8 to 20 amino acids in length. For example, (G4S)n can be used (n=1, 2, 3 or 4). In some aspects, the linker can be, but is not limited to, SGGGSGGSGSGS (SEQ ID NO:5).

In some aspects, the disclosed polypeptides can further comprise a nuclear localization signal (NLS). In some aspects, the NLS can be, but is not limited to, SV40 (PKKKRKV; SEQ ID NO:6), nucleoplasmin (AVKRPAATKKAGQAKKKKLD; SEQ ID NO:7), EGL-13 (MSRRRKANPTKLSENAKKLAKEVEN; SEQ ID NO:8), c-Myc (PAAKRVKLD; SEQ ID NO:9), TUS-protein (KLKIKRPVK; SEQ ID NO:10), or nucleoplasmin (KRPAATKKAGQAKKKK; SEQ ID NO:11). In some aspects, the NLS can be at the N-terminal or C-terminal end of the disclosed polypeptides.

In some aspects, the disclosed polypeptides comprising two heterologous polypeptide domains, wherein the first polypeptide domain comprises a Cas protein and wherein the second polypeptide domain comprises a zinc finger protein 865 (ZNF865) or fragment thereof wherein the polypeptide comprises the amino acid sequence of MDKKYSIGLAIGTNSVGWAVITDEYKVPSKKFKVLGNTDRHSIKKNLIGALLFDSGETA EATRLKRTARRRYTRRKNRICYLQEIFSNEMAKVDDSFFHRLEESFLVEEDKKHERHPIF GNIVDEVAYHEKYPTIYHLRKKLVDSTDKADLRLIYLALAHMIKFRGHFLIEGDLNPDN SDVDKLFIQLVQTYNQLFEENPINASGVDAKAILSARLSKSRRLENLIAQLPGEKKNGLF GNLIALSLGLTPNFKSNFDLAEDAKLQLSKDTYDDDLDNLLAQIGDQYADLFLAAKNLS DAILLSDILRVNTEITKAPLSASMIKRYDEHHQDLTLLKALVRQQLPEKYKEIFFDQSKN GYAGYIDGGASQEEFYKFIKPILEKMDGTEELLVKLNREDLLRKQRTFDNGSIPHQIHLG ELHAILRRQEDFYPFLKDNREKIEKILTFRIPYYVGPLARGNSRFAWMTRKSEETITPWN FEEVVDKGASAQ SFIERMTNFDKNLPNEKVLPKHSLLYEYFTVYNELTKVKYVTEGMR KPAFLSGEQKKAIVDLLFKTNRKVTVKQLKEDYFKKIECFDSVEISGVEDRFNASLGTY HDLLKIIKDKDFLDNEENEDILEDIVLTLTLFEDREMIEERLKTYAHLFDDKVMKQLKRR RYTGWGRLSRKLINGIRDKQSGKTILDFLKSDGFANRNFMQLIHDDSLTFKEDIQKAQV SGQGDSLHEHIANLAGSPAIKKGILQTVKVVDELVKVMGRHKPENIVIEMARENQTTQK GQKNSRERMKRIEEGIKELGSQILKEHPVENTQLQNEKLYLYYLQNGRDMYVDQELDI NRLSDYDVDAIVPQSFLKDDSIDNKVLTRSDKNRGKSDNVPSEEVVKKMKNYWRQLL NAKLITQRKFDNLTKAERGGLSELDKAGFIKRQLVETRQITKHVAQILDSRMNTKYDEN DKLIREVKVITLKSKLVSDFRKDFQFYKVREINNYHHAHDAYLNAVVGTALIKKYPKLE SEFVYGDYKVYDVRKMIAKSEQEIGKATAKYFFYSNIMNFFKTEITLANGEIRKRPLIET NGETGEIVWDKGRDFATVRKVLSMPQVNIVKKTEVQTGGFSKESILPKRNSDKLIARKK DWDPKKYGGFDSPTVAYSVLVVAKVEKGKSKKLKSVKELLGITIMERSSFEKNPIDFLE AKGYKEVKKDLIIKLPKYSLFELENGRKRMLASAGELQKGNELALPSKYVNFLYLASH YEKLKGSPEDNEQKQLFVEQHKHYLDEIIEQISEFSKRVILADANLDKVLSAYNKHRDK PIREQAENIIHLFTLTNLGAPAAFKYFDTTIDRKRYTSTKEVLDATLIHQSITGLYETRIDL SQLGGDKRPAATKKAGQAKKKKsgggsggsgsgsMEANPAGSGAGGGGSSGIGGEDGVHFQ S YPFDFLEFLNHQRFEPMELYGEHAKAVAALPCAPGPPPQPPPQPPPPQYDYPPQSTFKPK AEVPSSSSSSSSSSSSSSSSSSSSSSSSSQAKKPDPPLPPAFGAPPPPLFDAAFPTPQWGIVD LSGHQHLFGNLKRGGPASGPGVTPGLGAPAGAPGPLPAPSQTPPGPPAAAACDPTKDDK GYFRRLKYLMERRFPCGVCQKSFKQSSHLVQHMLVHSGERPYECGVCGRTYNHVSSLI RHRRCHKDVPPAAGGPPQPGPHLPPLGLPAPAASAATAAAPSTVSSGPPATPVAPAPSA DGSAAPAGVGVPPPATGGGDGPFACPLCWKVFKKPSHLHQHQIIHTGEKPFSCSVCSKS FNRRESLKRHVKTHSADLLRLPCGICGKAFRDASYLLKHQAAHAGAGAGGPRPVYPCD LCGKSYSAPQSLLRHKAAHAPPAAAAEAPKDGAASAPQPPPTFPPGPYLLPPDPPTTDSE KAAAAAAAVVYGAVPVPLLGAHPLLLGGAGTSGAGGSGASVPGKTFCCGICGRGFGR RETLKRHERIHTGEKPHQCPVCGKRFRESFHLSKHHVVHTRERPYKCELCGKVFGYPQS LTRHRQVHRLQLPCALAGAAGLPSTQGTPGACGPGASGTSAGPTDGLSYACSDCGEHF PDLFHVMSHKEVHMAEKPYGCDACGKTFGFIENLMWHKLVHQAAPERLLPPAPGGLQ PPDGSSGTDAASVLDNGLAGEVGAAVAALAGVSGGEDAGGAAVAGAGGGASSGPERF SCATCGQSFKHFLGLVTHKYVHLVRRTLGCGLCGQSFAGAYDLLLHRRSHRQKRGFRC PVCGKRFWEAALLMRHQRCHTEQRPYRCGVCGRGFLRSWYLRQHRVVHTGERAFKC GVCAKRFAQSSSLAEHRRLHAVARPQRCSACGKTFRYRSNLLEHQRLHLGERAYRCEH CGKGFFYLSSVLRHQRAHEPPRPELRCPACLKAFKDPGYFRKHLAAHQGGRPFRCSSCG EGFANTYGLKKHRLAHKAENLGGPGAGAGTLAGKDAPKKKRKVDYKDDDDK (SEQ ID NO: 12) wherein the bold sequence shown is the dCas9 sequence, the italicized sequence shown is a nucleoplasmin NLS sequence the lowercase sequence shown is the linker sequence, the underlined sequence shown is the ZNF865 sequence, the bold italicized sequence shown is a SV40 NLS sequence and the double underline sequence shown is a FLAG tag.

In some aspects, the disclosed polypeptides comprising two heterologous polypeptide domains, wherein the first polypeptide domain comprises a Cas protein and wherein the second polypeptide domain comprises a zinc finger protein 865 (ZNF865) or fragment thereof wherein the polypeptide comprises the amino acid sequence of MDKKYSIGLAIGTNSVGWAVITDEYKVPSKKFKVLGNTDRHSIKKNLIGALLFDSGETA EATRLKRTARRRYTRRKNRICYLQEIFSNEMAKVDDSFFHRLEESFLVEEDKKHERHPIF GNIVDEVAYHEKYPTIYHLRKKLVDSTDKADLRLIYLALAHMIKFRGHFLIEGDLNPDN SDVDKLFIQLVQTYNQLFEENPINASGVDAKAILSARLSKSRRLENLIAQLPGEKKNGLF GNLIALSLGLTPNFKSNFDLAEDAKLQLSKDTYDDDLDNLLAQIGDQYADLFLAAKNLS DAILLSDILRVNTEITKAPLSASMIKRYDEHHQDLTLLKALVRQQLPEKYKEIFFDQSKN GYAGYIDGGASQEEFYKFIKPILEKMDGTEELLVKLNREDLLRKQRTFDNGSIPHQIHLG ELHAILRRQEDFYPFLKDNREKIEKILTFRIPYYVGPLARGNSRFAWMTRKSEETITPWN FEEVVDKGASAQ SFIERMTNFDKNLPNEKVLPKHSLLYEYFTVYNELTKVKYVTEGMR KPAFLSGEQKKAIVDLLFKTNRKVTVKQLKEDYFKKIECFDSVEISGVEDRFNASLGTY HDLLKIIKDKDFLDNEENEDILEDIVLTLTLFEDREMIEERLKTYAHLFDDKVMKQLKRR RYTGWGRLSRKLINGIRDKQSGKTILDFLKSDGFANRNFMQLIHDDSLTFKEDIQKAQV SGQGDSLHEHIANLAGSPAIKKGILQTVKVVDELVKVMGRHKPENIVIEMARENQTTQK GQKNSRERMKRIEEGIKELGSQILKEHPVENTQLQNEKLYLYYLQNGRDMYVDQELDI NRLSDYDVDAIVPQSFLKDDSIDNKVLTRSDKNRGKSDNVPSEEVVKKMKNYWRQLL NAKLITQRKFDNLTKAERGGLSELDKAGFIKRQLVETRQITKHVAQILDSRMNTKYDEN DKLIREVKVITLKSKLVSDFRKDFQFYKVREINNYHHAHDAYLNAVVGTALIKKYPKLE SEFVYGDYKVYDVRKMIAKSEQEIGKATAKYFFYSNIMNFFKTEITLANGEIRKRPLIET NGETGEIVWDKGRDFATVRKVLSMPQVNIVKKTEVQTGGFSKESILPKRNSDKLIARKK DWDPKKYGGFDSPTVAYSVLVVAKVEKGKSKKLKSVKELLGITIMERSSFEKNPIDFLE AKGYKEVKKDLIIKLPKYSLFELENGRKRMLASAGELQKGNELALPSKYVNFLYLASH YEKLKGSPEDNEQKQLFVEQHKHYLDEIIEQISEFSKRVILADANLDKVLSAYNKHRDK PIREQAENIIHLFTLTNLGAPAAFKYFDTTIDRKRYTSTKEVLDATLIHQSITGLYETRIDL SQLGGDKRPAATKKAGQAKKKKsgggsggsgsgsMEANPAGSGAGGGGSSGIGGEDGVHFQ S YPFDFLEFLNHQRFEPMELYGEHAKAVAALPCAPGPPPQPPPQPPPPQYDYPPQSTFKPK AEVPSSSSSSSSSSSSSSSSSSSSSSSSSQAKKPDPPLPPAFGAPPPPLFDAAFPTPQWGIVD LSGHQHLFGNLKRGGPASGPGVTPGLGAPAGAPGPLPAPSQTPPGPPAAAACDPTKDDK GYFRRLKYLMERRFPCGVCQKSFKQSSHLVQHMLVHSGERPYECGVCGRTYNHVSSLI RHRRCHKDVPPAAGGPPQPGPHLPPLGLPAPAASAATAAAPSTVSSGPPATPVAPAPSA DGSAAPAGVGVPPPATGGGDGPFACPLCWKVFKKPSHLHQHQIIHTGEKPFSCSVCSKS FNRRESLKRHVKTHSADLLRLPCGICGKAFRDASYLLKHQAAHAGAGAGGPRPVYPCD LCGKSYSAPQSLLRHKAAHAPPAAAAEAPKDGAASAPQPPPTFPPGPYLLPPDPPTTDSE KAAAAAAAVVYGAVPVPLLGAHPLLLGGAGTSGAGGSGASVPGKTFCCGICGRGFGR RETLKRHERIHTGEKPHQCPVCGKRFRESFHLSKHHVVHTRERPYKCELCGKVFGYPQS LTRHRQVHRLQLPCALAGAAGLPSTQGTPGACGPGASGTSAGPTDGLSYACSDCGEHF PDLFHVMSHKEVHMAEKPYGCDACGKTFGFIENLMWHKLVHQAAPERLLPPAPGGLQ PPDGSSGTDAASVLDNGLAGEVGAAVAALAGVSGPKKKRKVDYKDDDDK (SEQ ID NO: 13) wherein the bold sequence shown is the dCas9 sequence, the italicized sequence shown is a nucleoplasmin NLS sequence, the lowercase sequence shown is the linker sequence, the underlined sequence shown is a fragment of ZNF865 having amino acids 1-769 of the ZNF865 sequence, the bold italicized sequence shown is a SV40 NLS sequence, and the double underlined sequence shown is a FLAG tag.

In some aspects, the disclosed polypeptides comprising two heterologous polypeptide domains, wherein the first polypeptide domain comprises a Cas protein and wherein the second polypeptide domain comprises a zinc finger protein 865 (ZNF865) or fragment thereof wherein the polypeptide comprises the amino acid sequence of: MDKKYSIGLAIGTNSVGWAVITDEYKVPSKKFKVLGNTDRHSIKKNLIGALLFDSGETA EATRLKRTARRRYTRRKNRICYLQEIFSNEMAKVDDSFFHRLEESFLVEEDKKHERHPIF GNIVDEVAYHEKYPTIYHLRKKLVDSTDKADLRLIYLALAHMIKFRGHFLIEGDLNPDN SDVDKLFIQLVQTYNQLFEENPINASGVDAKAILSARLSKSRRLENLIAQLPGEKKNGLF GNLIALSLGLTPNFKSNFDLAEDAKLQLSKDTYDDDLDNLLAQIGDQYADLFLAAKNLS DAILLSDILRVNTEITKAPLSASMIKRYDEHHQDLTLLKALVRQQLPEKYKEIFFDQSKN GYAGYIDGGASQEEFYKFIKPILEKMDGTEELLVKLNREDLLRKQRTFDNGSIPHQIHLG ELHAILRRQEDFYPFLKDNREKIEKILTFRIPYYVGPLARGNSRFAWMTRKSEETITPWN FEEVVDKGASAQSFIERMTNFDKNLPNEKVLPKHSLLYEYFTVYNELTKVKYVTEGMR KPAFLSGEQKKAIVDLLFKTNRKVTVKQLKEDYFKKIECFDSVEISGVEDRFNASLGTY HDLLKIIKDKDFLDNEENEDILEDIVLTLTLFEDREMIEERLKTYAHLFDDKVMKQLKRR RYTGWGRLSRKLINGIRDKQSGKTILDFLKSDGFANRNFMQLIHDDSLTFKEDIQKAQV SGQGDSLHEHIANLAGSPAIKKGILQTVKVVDELVKVMGRHKPENIVIEMARENQTTQK GQKNSRERMKRIEEGIKELGSQILKEHPVENTQLQNEKLYLYYLQNGRDMYVDQELDI NRLSDYDVDAIVPQSFLKDDSIDNKVLTRSDKNRGKSDNVPSEEVVKKMKNYWRQLL NAKLITQRKFDNLTKAERGGLSELDKAGFIKRQLVETRQITKHVAQILDSRMNTKYDEN DKLIREVKVITLKSKLVSDFRKDFQFYKVREINNYHHAHDAYLNAVVGTALIKKYPKLE SEFVYGDYKVYDVRKMIAKSEQEIGKATAKYFFYSNIMNFFKTEITLANGEIRKRPLIET NGETGEIVWDKGRDFATVRKVLSMPQVNIVKKTEVQTGGFSKESILPKRNSDKLIARKK DWDPKKYGGFDSPTVAYSVLVVAKVEKGKSKKLKSVKELLGITIMERSSFEKNPIDFLE AKGYKEVKKDLIIKLPKYSLFELENGRKRMLASAGELQKGNELALPSKYVNFLYLASH YEKLKGSPEDNEQKQLFVEQHKHYLDEIIEQISEFSKRVILADANLDKVLSAYNKHRDK PIREQAENIIHLFTLTNLGAPAAFKYFDTTIDRKRYTSTKEVLDATLIHQSITGLYETRIDL SQLGGDKRPAATKKAGQAKKKKsgggsggsgsgsGEDAGGAAVAGAGGGASSGPERFSCATC GQSFKHFLGLVTHKYVHLVRRTLGCGLCGQSFAGAYDLLLHRRSHRQKRGFRCPVCG KRFWEAALLMRHQRCHTEQRPYRCGVCGRGFLRSWYLRQHRVVHTGERAFKCGVCA KRFAQSSSLAEHRRLHAVARPQRCSACGKTFRYRSNLLEHQRLHLGERAYRCEHCGKG FFYLSSVLRHQRAHEPPRPELRCPACLKAFKDPGYFRKHLAAHQGGRPFRCSSCGEGFA NTYGLKKHRLAHKAENLGGPGAGAGTLAGKDAPKKKRKVDYKDDDDK (SEQ ID NO:14) wherein the bold sequence shown is the dCas9 sequence, the italicized sequence shown is a nucleoplasmin NLS sequence, the lowercase sequence shown is the linker sequence, the underlined sequence shown is a fragment of ZNF865 having amino acids 770-1059 of the ZNF865 sequence, wherein the bold italicized sequence shown is a SV40 NLS sequence, wherein the double underlined sequence shown is a FLAG tag.

Also disclosed are variants of the disclosed are polypeptides. In some aspects, the Cas protein or ZNF865 protein can be a variant of any known Cas protein or any ZN865 protein or fragment thereof disclosed herein. In some aspects, a variant of the disclosed polypeptides retains its functional activity for both the Cas protein and the ZNF865 protein.

### C. Nucleic Acid Sequences

Disclosed are polynucleotides capable of encoding one or more of the disclosed polypeptides. Thus, disclosed are polynucleotides capable of encoding a polypeptide comprising two heterologous polypeptide domains, wherein the first polypeptide domain comprises a Cas protein and wherein the second polypeptide domain comprises ZNF865 or fragment thereof. In some aspects, the polynucleotides can be referred to as nucleic acid constructs.

In some aspects, the nucleic acid sequence that encodes ZNF865 is present on the N-terminal or C-terminal end of the nucleic acid that encodes a Cas protein. Thus, in some aspects, the polynucleotide can be a sequence that encodes Cas protein-ZNF865 or a sequence that encodes ZNF865-Cas protein.

In some aspects, the nucleic acid sequence that encodes ZNF865 can be a full length nucleic acid sequence that encodes ZNF865 or can be a fragment thereof. In some aspects, the nucleic acid sequence that encodes the full length ZNF865 comprises the nucleic acid sequence: ACTTCCGGTCGGGCCCTCGGGTCTCCCCGGAGCGGCGGCGCCTCCTCCGCCTCCTCG GCCTCCTCCCGGCGGAGACCCCGGCGCCGgtgagtgacggggtgcgtggcccgggggcccgggtgcagcg ggggcggggggccccgccctggcccgctgtggtccccagaacgtcccccaacccctagcaagtcagtgcagctcccgccgggccctt gccaagggacccccaatcactgctccccggtaggatcactgcagatagctcccccatgaacgcccccagctaggatggctgccaaagcg ctttcattcgtggcgatcccctaaacggctgccagggcccccaaatgatacctcctcgtaggatcccctccacatagcgctccccgtcattgc ttcctccaggtcagcctccagatgccatcagcattgcccctccccatgtagggcagctgctcgtgaaccctcttattagtatcctctatataatt ctccccagagtgtttccttagtgccttgcagcagaactgtcccccgttatctctgtccatacagcaacagcccccaatggccctgaccacctc cctccccagcagaacgccccttcgtgggtgtgaaaatactttctattctggtcagcaccaagaatgccttttcccttctgcaggtcctccagtg attccccttaagaatgcccctttcaaagccacccccccatcgcagcggcacagctccctctagagttccttcacactcacatcctctcccgcc tcaggtagaaatatccgcctgcttagctccaggctcccatgacatactcccgtacctcctctcaccccaccctcatcgcggtcagcccgtctt cattacttctgccacagaacagtgtcccgcagtgaggcggtgaagccttccttcccagaatgtgcctcatcctcttcctatggcgtgaacaac tgttgccctgacctgcagcttctcacccagctctcaggctatcgtcctggactccctagggaagaccctggacttcactagggtgtgacttctt ttctcgtaggcattccttctgcgttgaacgcatattcactattctagctgaagggtataatatacagccacgaagggggtcgatacacacagtg ttctcactgtgcggggtctcacagtctagttgatcagacacgagtcgacaaagatacacggggttttgtgggttctctgagagcctgatgaac tactttagaggtgaaagctgaattacaagtggcagcttagccatgcaaagatctgggtgagcaggcagcaagaacaacaagtgcaaaggc cctgaagtggcggtgagcacggtgagtggaggagcagcaggggaggccggtgagggtggagtggaagatttgaaggcaagaacaag aggaaggaagctttggtctcaaaaggctttggaggtgagggcactgggataaattatctagcctttgtcagctctaccgagcccagaatgttc ttcattcattcatacgttcattcgtaaacactggttgaacaactgtttaatgagcacctactttacattgggggtacaacagagaacaaaataga caagaatccctgtcctcatggagtggatatgctagttgagggggcagatgattaaaagacaaaatacaggccaggcgaggtggctcatgc ctgtaatcccagcagttgggaaggccaaagtgggaggatcgctggaggccaggagttcaagaccaacctcggcaacatagtgagaccct gtctttacaggaaaaaaaaaaaaaaaaaaaaaaagctgggtgtggttggtgggcacctgtagtccccagctattcaggaggctgaggtgg gagggctgcttgagcccaggagttcgaggctgcagtgaaaaaaaataaaaacaaaaaccaagagtatatgtgcgtgttcagaatatcacg ggataaagagatagacctggggggctgtggttcaggatggcctcccccaggattttcctatgtcttgtcatcttgtccctcccaaaagccaca caccaaaaagttatacaagagtaaaaattccccttctcctggacaactgtggcccacttgggggctgctcttaacagcgccctaaccccagg agcgaagtgattgggaggatgagccctggattccactagggttcacagctttgagcaaatcactccctcctttcctagcctcattcattcacttc ttcaaacaaaaatgtatcaagccaggaactgttgtaggtcctggggatggaacagtgagcaagagataaaaatccttgccttcaggaagcc gatcatctggatggggagaccaacaaaatcaaggcaaagaagtagattatgtgaggttataaatgatgctgagcagtaaggagagaaatg aagcagggttagggagatgaggatgtgggcctgggtgacaggtcattggttactgagaggctcattgagaagacagttgaccaaagatgg ggaggaggtgaaggggtgagcgagatggttgcctgagagtcagaatattacaggcaacagcaacagccagcgcaaaggccctgcagc aggaatagagtgggcacatgggaagagcagccaggagactgtggaggtcattgtaaggactgtggctttttttttttttttttttgaaaaggagt ttcgctcttgttgcccaggctggagtgcagtggtgtgatcttagctcgctgcaacctccgcctcccgggttcaagtgattctcctgcctcagcc tcccgagtagctgggattacaggcatgtgccaccacgcccagctaattttgtatttttagtagagatggggttctccatgttggtcaggctggt ctcaaactcccgacctcaggtgatccgcctgccttggcctccaaaaatgctgggattacaggcgtgagccaccgtgcccagtcaggactgt ggcttttgaatgagatggggttactgtttggtttgggaccgaggatagacatgctttggttatagtcctcctgtcagtaaaatgggaataataat aacacatttgttgtgaagtttgaatgaaacaagccacataaggcacctaagtacaatgcttgttagaggtcaacaaatgttggtgacttgttctg gtcttgggtcaagccaagagacttgggatttgttggcagggagatgggtggagcaatgcatgtttccagtatgaagtgggattgggggcgg gggtcctccttcattttggagggagaaataccgaaagacagcatgtgatgggactctggagccttgctgccttcttcaaccactggccctgc cactagttaacaatgtgactttagaagtttgtttgtttgtttttgagaaacagagtctcactctgtcacccaggctggagtgcagtggcgtgagct cactgcaacctctgtgtcctgggttcaagcaattatcctgcctcagcttccccactagctggaactacaggtgtgcaccaccacacccggcta atttttttatttttagtagagacggggtttcactatatattggccaggctagtctcgaactcctgactatagttgatccacccacctcggcctccca aagtgctggcattacaggcatgagccaccatgcctggcctgactttggaagtttaatttatctctctggccctcagtctcttcctttgtatttcaga gtcaatgcttgtaaccatcttctagggttactgggaggattcgttgagctcacacacatcagagttacaagagtgctgggctggtagaaagca cacatccagtgttatatgcgcacgttactctttattaacaaaccagaatttcatcacaaggcagcgttgttgcagcagaacggcccacagtctg gcatcagaaagacctggaatcagttacctgcttcctctttattggccacatgaccttggaggaatgccttccccatgctgctcctcagtttccc cacttataagatgggcccagtcatggtacaggactgtgctgcctccaggcaataacaaatatcaaactggtcaaacaataaatgtgaaccgt taatacacttgctgtgggactcctgtcccttggtggtggtggtggaggggtgggtccctccccatcatgcttctctctgcataaaaccctcctat gacatcccctcttaaaaagataccagtttaggcttgagttgtatgaaagagggaatgtttctggtttcctagtggagattatcataagcctgatc aattctgtccaccactgatgtatccccttcagcatctccacaccctgccctcagtgccctcctcataacatcactgtccccagaattgcttctcc caggaattcctgggatcatttagctccatttctgctacatgtccccctccgttcctctgggagtttgatcagcttagtctcatcgctttgttcattcc acaaataccaagatagacattcacagctgtgcatttccctctgagcattgctttggctgcacctcctcagctttggtatgttgtgttttcattttcatt aattgctaagcagtttttttgctttttgtttgtttgtgtttttgagatggagtcgcgctcttgttgcccaggctggagtgcagtggcacgatctcagc tcactgcaacctccacctcccgagttcaagcgattctcctgcctcagcctcccaagtagctgggattacaggcgcccaccaccaagcctgg ctaatttttgtatttttagtagagacggggtttcgccatgtaggccagactggtgtcgaactcctaacctcaggtgatcctcctgccttggcctcc caaagtgccgggattacaggtgtgggccaccgcacctggccagcattttctaatgtatgatttctttgtggacccattggtaatttaggaaggt ggtgtttccttttcacataccagcagatatttgtcaagcacccacactgggccagtgctacgctaagtgctggggtcacaggggcgagcaag acaggccaagtccttgcttcctagagaggggccaaacagtaaacacacagaaaaggaatgacatgagttggtggcaataactataatgaa acacctgcggctggatgagggactggagagtgatggggtggggagtgctgagtagagacccaaggaggctccagagggagtcttgttg aaatctggcgaagaacactgtaggcagaagagaaagtaaccaccaccctctcccaccaggccccttgtatagacacccctcataacccag aagacatccctaaataaagctcttatcctgatagttctcccctcacctttgttcactcggggaacagcccagtgaaacactgctgtgggggca agggtgggaatggggttcaagcccattgatccagactcctactccgtgaccatggcaactcactttgcctctccatgccttagttttctaatttat ccaacggcgggaacagctgcccctatttagagttgtggggagtcagtgaattaatatgtgtaacgtgcttagaaccgtggcgggcctctag gagatgtccagtcagtgacagctacgattaacctgtgctctagtggagacaagctagtcaggggcttctttccccaaacactcttgccctccc tccccatagcgccctaaatcaccccctattccctgcatccttatggcctgtgtttgcctccctggccagtcagccccctccagagctgtctccc caacttaggttccacctacagccttccaattaccaccccagccaagcctgtcctctttccggagatcagattaaacggagggggtgtgaagg gcatgggatgttgagtcttgtccaaggacaaggacacccggagctggagactaaaaatatctcactccaggatggcgtttggggtggggg tggaagagacactcacccctcacctgtgggaggagagagcagcccgatggatagaggataaagggtccaggccttggtaggaaccccc ttgacccccatagcgggctgggcttagagcctggcccccatcgggtagggatgatggacaggaagcgggtcagacctcatgggcccttc accgactcaccctccgcccacttggtggaggtgcttccccaggccagcaagtagaccaagaactcggagatcagttacgggtgctgagg gtctggtgtggtgtggggtacacacatggatacctgccatctggaatgaaaagtcgtgtaaaggaggtgccagagaatgatgccgggggt ggagggtcaggagaagccccactaagggcaaggacatgggggggtcaggagaagccccactaagggcaaggatggaactcaagctc cctgcttctgttgctaggcttagcccaggtgtgtgatgtcatggatggtacaaatagctcagggggttgaatgcattctgctgatgcatgtgttt cattatgttctgttccaaccctgttgaatctccctgcggtataggggttcccccattttaaacatggagaaagtgtggatcccagtggagatgg gatttgaacccaggtctgtccaagcccagggtctgtgttttgattgtatcagaccaccttctgactatatgccagcccccttgctggaatttgttt ctcccaggcaagcaaattctcctttctactgtctattaatattaaaagagtgcactccccatactgacttttttcagttctctaggcttgtcctgactt ccccatttctactactagtagtaacagtgataacagtatcgtatcttcaattttttaattttagtttttagagccaaggtcttgctctgtcacccaggc tggagtacagtgatgtgatcacagctcattgtagccttcacctcccgggctcaagcagctctcctgcctcagcctcccaaagtgctgggatg acagctgtcagccactgtgcccagtccataccttctatgtacggagtgcttacagagtgccaggaaccgccccctaagagccttgcacatat gtgtgtgttgaatcctcacagtcctgtgaggtaggcatgtgctttgtggtccccaggtgtagatgaggaaatggaggcacagagaggttcag tcacttgcccgtggccacacagctagaagtggcagggcttacatttgattctggccccagagtcctccctcctaattacgctgatagaaactg tgattccccttatcattatgctcattttaggataaaacctgaagggatgaggacggagggctgtgggtggggtagggtgcaggagtatggag agacagcagagggtctgagagggtttggaaagaaaactgagtccagaggcccccaggatgcttggacaattggagtctgaaaaggaga gaaagtagagacggcccaggccagcgcgctggctcacatctgtaatcacccagcactttgggaggtggaggctggaggatggcgtgag tccaggagtttcagaccagcctgggcaacaaagcaaaaccccgtttctacaaataatagagggcctgcaggtggtgtggaggagagggt cccctctacaaagctgtgtcaggaccccagaaaagatggggagagatggaaggaattagagaaggaaaagaggtggggagtgggtgg gagttgagaacacagagagggaaggtgaaggtgagtaggagagtcctggggccagggagccaagaggaagggctgccctttggggt ggacaggcagagggactggaagcctaaatctggaaggctagggggtgatccgcacaaggatggatgagtgggcgcacagctccattca cctgcggggagacccagcgccgcggccgtgtcctcagccccgtcctcctcttcacagGGTCTCCCGTCTCCCACCCGC CGGAGATGGAGGCGAACCCAGCGGGCAGCGGCGCCGGGGGTGGCGGGAGCAGCGG CATCGGGGGCGAGGACGGGGTGCACTTCCAGAGCTACCCCTTCGACTTCCTGGAAT TCCTCAACCACCAGCGCTTCGAGCCCATGGΔΔCTGTATGGGGAACACGCCAAGGCG GTGGCGGCCCTGCCCTGCGCCCCCGGCCCCCCGCCGCAGCCCCCGCCGCAGCCCCCT CCCCCGCAGTATGACTACCCGCCCCAGTCCACCTTCAAGCCCAAGGCGGAGGTGCC CTCCTCGTCCTCGTCCTCGTCCTCCTCCTCCTCCTCTTCGTCCTCCTCGTCGTCATCTT CGTCCTCTTCCTCTTCCCAAGCCAAGAAGCCCGATCCGCCCCTGCCGCCCGCCTTCG GGGCGCCCCCTCCTCCCCTCTTTGACGCTGCTTTCCCCACTCCGCAGTGGGGCATCG TGGACCTCTCGGGGCACCAGCACTTGTTTGGGAACCTGAAGCGAGGAGGGCCCGCG TCCGGGCCGGGGGTGACGCCTGGGCTGGGCGCTCCCGCGGGGGCCCCAGGGCCGCT TCCTGCCCCCTCGCAGACCCCGCCAGGACCCCCCGCGGCGGCGGCCTGCGACCCCA CCAAGGACGACAAGGGCTACTTCCGGAGACTGAAGTACCTGATGGAGCGGCGCTTC CCCTGCGGCGTGTGCCAGAAGTCCTTCAAGCAGTCCTCGCACCTGGTCCAGCACATG CTGGTGCACTCGGGGGAGAGGCCCTACGAATGCGGCGTCTGCGGCCGCACCTACAA CCACGTGTCCAGCCTCATCCGCCACCGCCGCTGCCACAAGGACGTGCCACCGGCCG CGGGGGGCCCGCCCCAGCCCGGCCCCCACCTCCCGCCGCTGGGCCTCCCAGCACCC GCTGCCAGCGCCGCCACCGCCGCCGCCCCCTCCACGGTGTCCTCGGGCCCTCCAGCC ACGCCCGTGGCGCCTGCCCCCTCCGCAGACGGGAGCGCCGCCCCTGCTGGTGTTGG GGTGCCCCCTCCTGCCACCGGGGGTGGCGATGGCCCGTTCGCCTGCCCACTCTGCTG GAAGGTTTTCAAGAAGCCCAGTCACCTCCACCAGCACCAGATCATCCACACGGGCG AGAAGCCCTTCTCCTGCTCCGTGTGCAGCAAAAGCTTCAACCGCAGGGAGAGTCTG AAGCGCCACGTGAAGACGCACTCGGCCGACCTCCTGCGCCTGCCCTGCGGCATCTG CGGGAAGGCCTTCCGCGACGCCTCCTACCTCCTCAAGCACCAGGCGGCCCACGCGG GGGCGGGCGCCGGGGGGCCTCGGCCCGTGTACCCCTGCGACCTGTGCGGCAAGTCC TACTCGGCTCCGCAGAGCCTGCTCCGCCACAAGGCCGCCCACGCCCCGCCCGCTGC CGCTGCGGAGGCGCCCAAGGACGGGGCGGCCTCGGCCCCGCAGCCCCCGCCCACCT TCCCCCCGGGCCCGTACCTCCTGCCCCCCGACCCTCCCACCACAGACAGCGAGAAG GCGGCGGCGGCCGCGGCGGCGGTGGTGTACGGCGCTGTGCCCGTCCCGCTCCTGGG CGCCCACCCGCTGCTGCTCGGCGGCGCGGGGACCAGCGGGGCGGGAGGCTCGGGC GCCAGCGTCCCAGGAAAGACGTTCTGCTGCGGCATCTGCGGGCGCGGCTTCGGGCG CCGCGAGACCCTGAAGCGCCATGAGCGCATCCACACGGGCGAGAAGCCCCACCAGT GCCCCGTGTGTGGGAAGCGCTTCCGCGAATCCTTCCACTTGAGCAAGCATCACGTG GTGCACACGCGCGAGCGGCCCTACAAGTGCGAGCTCTGCGGCAAGGTCTTCGGCTA CCCGCAGAGCCTCACCCGCCACCGCCAGGTGCACCGGCTCCAGCTGCCCTGCGCCC TGGCCGGGGCAGCCGGCCTCCCCTCCACCCAAGGCACACCGGGGGCCTGTGGGCCC GGGGCCTCGGGCACGTCTGCAGGGCCCACCGATGGGCTGAGCTACGCCTGCTCGGA CTGCGGCGAGCACTTCCCGGATCTCTTTCACGTCATGAGTCACAAGGAGGTCCACAT GGCAGAGAAGCCATACGGCTGCGACGCCTGCGGCAAGACCTTCGGCTTCATCGAGA ACCTCATGTGGCACAAGCTGGTCCACCAGGCCGCCCCCGAGCGCCTGCTCCCGCCC GCACCCGGCGGCCTGCAGCCCCCGGACGGCTCCAGCGGCACGGATGCGGCCAGCGT GCTGGACAACGGGCTGGCGGGGGAGGTGGGGGCGGCCGTGGCGGCACTGGCAGGG GTGTCTGGGGGTGAGGACGCAGGCGGGGCGGCGGTGGCAGGTGCTGGCGGGGGTG CCAGTTCCGGCCCCGAGCGCTTCAGCTGTGCCACGTGCGGCCAGAGTTTCAAGCACT TCCTGGGCCTCGTGACTCACAAGTACGTGCACCTGGTGCGACGGACCCTGGGCTGC GGCCTCTGCGGCCAGAGCTTCGCGGGCGCCTACGACTTGCTCCTACACCGCCGCAG CCATCGGCAGAAGCGGGGTTTCCGCTGCCCGGTGTGCGGGAAGCGCTTCTGGGAGG CGGCCCTGCTGATGCGCCACCAGCGCTGCCACACGGAACAGCGGCCGTACCGATGT GGCGTGTGCGGCCGAGGCTTCCTGCGCTCCTGGTACCTGCGGCAGCACCGCGTGGT GCACACTGGCGAGCGGGCCTTCAAGTGCGGCGTGTGCGCCAAGCGCTTCGCGCAGT CGTCCAGCCTGGCAGAGCACCGGCGGCTGCACGCTGTGGCCCGGCCCCAGCGCTGC AGCGCCTGTGGCAAGACCTTCCGCTACCGCTCCAACCTGCTGGAGCACCAGCGGCT GCACCTGGGCGAGCGCGCCTACCGCTGTGAGCACTGCGGCAAGGGCTTCTTCTACC TGAGCTCCGTGCTGCGCCACCAGCGCGCCCATGAGCCGCCGCGGCCCGAGCTCCGC TGCCCCGCCTGCCTCAAGGCCTTCAAGGATCCCGGCTACTTCCGTAAGCACCTGGCT GCCCACCAGGGCGGCCGGCCCTTCCGCTGCTCCTCCTGCGGCGAGGGCTTCGCCAA CACCTACGGCCTCAAGAAACACCGCCTGGCGCACAAGGCCGAGAACCTCGGGGGG CCTGGAGCAGGGGCGGGCACCTTGGCCGGGAAGGATGCCTGACCGAGGGGTTCCCA TCCCACTCCCATCAAAAGCCCCCTTCTGGACTCCCACCTCCCAGGACTGATCAGACT CTTCCCCCCTCCTCGCTGTTGCCCCATCCTTCAGΔΔCTTCACACGGACTGGCGACCTT CAGGGCGCACGCCCGACAGGCTCAAGACTGAATCACTCCCATCCTCGACCTCTCTG CCCTCCCCTCATCCCATCAGACACTGAACCCTATCCTCCGTCCAACCCTCGTTTGTG ACCCGCATCAGCCCCCGCCCCAGCAGCACTCTGCCCCCAGTAAGTTTTGGCGGAGA TGGGTCTGAACCGCCCCTCCCCCTCCTTTGGAATCTGGCTGGACAGTGGAGTATGAG CAGAGTTGGGAGGGCACAAGGGAGTGCTGGGTGCTTTTTGGGGTGGGGGGGTGGG GGGCGGGGTGGCAGACGCGGCTTGTACAGAGCGGAGAATAATAAATCTTACCATGA GGGCC (SEQ ID NO:463). In some aspects, the nucleic acid sequence that encodes the fragment of ZNF865 comprises the nucleic acid sequence comprise or consist of a fragment of SEQ ID NO:463.

### D. Vectors

Disclosed are vectors comprising a nucleic acid sequence that encodes one or more of the disclosed polypeptides. Disclosed are vectors comprising a nucleic acid sequence that encode only ZNF865, a fragment of ZNF865, a variant of ZNF865, any of the Cas-ZNF865 fusion proteins disclosed herein, or a ZNF865-specific gRNA.

In some aspects, the vector is an expression vector. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

### 1. Vectors Comprising ZNF865 Nucleic Acid Sequence

Disclosed are vectors comprising a nucleic acid sequence that encodes ZNF865. Disclosed are vectors comprising a nucleic acid sequence that encodes a variant of ZNF865. Disclosed are vectors comprising a nucleic acid sequence that encodes a fragment of ZNF865. In some aspects, the nucleic acid sequence that encodes ZNF865, a variant of ZNF865 or a fragment of ZNF865 can be one or more of the nucleic acid sequences described herein.

In some aspects, the vectors are viral vectors, such as, but not limited to, lentiviral vector, adenoviral vector, or adeno-associated viral vector.

### 2. Vectors Comprising Cas and ZNF865 Nucleic Acid Sequences

Disclosed are vectors comprising one or more of the polynucleotides (e.g. Cas-ZNF865) disclosed herein. For example, disclosed are vectors comprising a polynucleotide capable of encoding one or more of the disclosed polypeptide. In some aspects, disclosed are vectors comprising a polynucleotide capable of encoding a polypeptide comprising two heterologous polypeptide domains, wherein the first polypeptide domain comprises a Cas protein and wherein the second polypeptide domain comprises ZNF865, a variant of ZNF865 or a fragment of ZNF865.

In some aspects, the vector comprises a nucleic acid sequence that encodes ZNF865, a variant of ZNF865 or a fragment of ZNF865 N-terminal or C-terminal to the nucleic acid that encodes a Cas protein. Thus, in some aspects, the polynucleotide can be a sequence that encodes Cas protein-ZNF865 or a sequence that encodes ZNF865-Cas protein.

In some aspects, the disclosed vectors can further comprise a nucleic acid sequence encoding a guide RNA (gRNA). Thus, the disclosed vectors can comprise both a gRNA and a nucleotide sequence capable of encoding a one or more of the disclosed polypeptides in the same vector.

In some aspects, the gRNA sequence is capable of binding to a target site of a target gene therefore bringing the disclosed polypeptide expressed from the disclosed vectors to the area of the target gene and allowing the ZNF865, variant of ZNF865 or fragment of ZNF865 to boost transcriptional activity of the target gene. In some aspects, the target gene can be any gene. In some aspects, the target gene can be ACAN, Col2A, IL-2, or IFN-γ.

In some aspects, the vectors are viral vectors, such as, but not limited to, Lentiviral vector, adenoviral vector, or adeno-associated viral vector.

3. Vectors Comprising ZNF865-Specific gRNA

Disclosed are vectors comprising a nucleic acid sequence of a ZNF865-specific gRNA. In some aspects, the ZNF865-specific gRNA can be any of those listed in Table 1. Table 1: Examples of ZNF865-specific gRNA (top to bottom: SEQ ID NOS: 164 in first column, SEQ ID NOS: 165-314 in second column; SEQ ID NOS: 315-462 in third column) Table 1.

TABLE 1

| | | |
|---|---|---|
| GTCAGGACCCCAGAAAA | CTGGGTTTGGCTGTTTGG | TGGGTCTCCCCGCAGGT |
| GAT | GATGG | GAATGG |
| GCGCACAAGGATGGATG | GTCTCCCGTCTCCCACCC | AGGGACTGGAAGCCTAA |
| AGT | GCCGG | ATCTGG |
| GGGGACTGGAAGCCTAA | CTGTGAGATGGGCGTGG | CGCACAGCTCCATTCAC |
| ATC | TTCAGG | CTGCGG |
| GGGGGTGATCCGCACAA | GCGCCGGGGTCTCCGCC | GGGGGTGATCCGCACAA |
| GGA | GGGAGG | GGATGG |
| GTCTGCCTGTCCACCCC | GGAGAAACTCTGAGGGA | AAGCCTAAATCTGGAAG |
| AAA | ACAGGG | GCTAGG |
| CCGCACAAGGATGGATG | CACGCCGGCGCCTCGGG | TCCAATTGTCCAAGCAT |
| AGT | AACGGG | CCTGGG |
| GACGCCCAGAGCGTGTC | AGAGTTTCTCCTTTAAGC | GAGGACACGGCCGCGGC |
| GCG | CCCGG | GCTGGG |
| GAGGCGGGCATTCAAAG | CGTGGTTCAGGGCCAGG | GTACATAGAAGGTATGG |
| CGC | GGGCGG | ACTGGG |
| TCGCCCACCGGAATCGG | GTGGGCGTGGTTCAGGG | TGTGCAAGGCTCTTAGG |
| CCC | CCGAGG | GGGCGG |
| ATCCTCCACGCCGGCGC | CTGGAACTTCACCCCTCC | TCCTGACACAGCTTTGTA |
| CTC | CTAGG | GAGGG |
| ACTTCCGCTTCCGGGCG | AGGAGGGGCTGAATGGG | AGAGAAAGTAGAGACG |
| GGC | TCTGGG | GCCCAGG |
| gcACTTCCGGTCGGGCCC | GGAATAGAAGCCCTGGG | TGGACAATTGGAGTCTG |
| TC | GTTGGG | AAAAGG |
| GACGCCCAGAGCGTGTC | TGCATGCTGGTCAAGAG | GGCTGCCCTTTGGGGTG |
| GCGGGG | CCATGG | GACAGG |
| TCGCGGGGTTCCCGTAG | GACAGACTTTGAGGCAG | CCCCAGGATGCTTGGAC |
| AAGAGG | TAGGGG | AATTGG |
| GAGGCGGGCATTCAAAG | TTATAATCACGCTGTGA | AAGGTGAGTAGGAGAGT |
| CGCAGG | GATGGG | CCTGGG |
| TCGCCCACCGGAATCGG | CCACGCCGGCGCCTCGG | CACAGCTCCATTCACCT |
| CCCGGG | GAACGG | GCGGGG |
| TTCGCCCACCGGAATCG | CCCAGGGCTTCTATTCCG | GAAGGTGAGTAGGAGAG |
| GCCCGG | CATGG | TCCTGG |
| ATGGGGGCGCACGACTG | TGGGTCTGGGTTTGGCT | AAAGCACATGCCTACCT |
| CTATGG | GTTTGG | CACAGG |
| CGCGACACGCTCTGGGC | GGGCGTGGTTCAGGGCC | AATTAGGAGGGAGGACT |
| GTCCGG | AGGGGG | CTGGGG |
| AGCCGGAGGTGTGGCTT | GGCTGCCTGCTCAGACC | CGGGAGACCCTGTGAAG |
| AACCGG | ATGCGG | AGGAGG |
| CGACTGCTATGGAGGCG | AGAAACTGCATGAGGGC | GTGGTCCCCAGGTGTAG |
| ATGGGG | GAGGGG | ATGAGG |
| AAGCCCGGGCCGATTCC | GAGTGGAAGCTTAGCAG | CATTTTAGGATAAAACC |
| GGTGGG | GAGGGG | TGAAGG |
| GGAAGTCGCGCCCGTTC | TAAAAGAATGCTTTAGA | AGGTGAGTAGGAGAGTC |
| CCGAGG | GGTGGG | CTGGGG |
| ACGACTGCTATGGAGGC | CGGAAAGAGGAGGCGGT | GCCCCGTCCTCCTCTTCA |
| GATGGG | GGCGGG | CAGGG |
| ACTTCCGCTTCCGGGCG | GGGTCTGGGTTTGGCTG | GCACAGCTCCATTCACC |
| GGCGGG | TTTGGG | TGCGGG |
| GTTAAGCCACACCTCCG | CGGAGGAGGCGCCGCCG | GTAATTAGGAGGGAGGA |
| GCTCGG | CTCCGG | CTCTGG |
| CTCAGTTCGCCCACCGG | GATTTAAAAGAATGCTT | AAGAGGAAGGGCTGCCC |
| AATCGG | TAGAGG | TTTGGG |
| GGAACCCCGCGACACGC | CCGCTTCCGGGCGGGCG | ACCTGAAGGGATGAGGA |
| TCTGGG | GGCAGG | CGGAGG |
| GGGGCGCACGACTGCTA | TGGTTCAGGGCCAGGGG | AGACGGGAGACCCTGTG |
| TGGAGG | GCGGGG | AAGAGG |
| CGGACGCCCAGAGCGTG | AAGGGAAGTTCCTGGGA | AACTGAGTCCAGAGGCC |
| TCGCGG | GAGTGG | CCCAGG |
| TGCGCTTGCGCACTGAC | AGTTCCAGAGAGTGGGA | CGGGGCTGAGGACACGG |
| TCGGGG | GCTTGG | CCGCGG |
| GTCCGGTTAAGCCACAC | AATAGAAGCCCTGGGGT | GTCTCCCGTCTCCCACCC |
| CTCCGG | TGGGGG | GCCGG |
| GGACGCGAGCAGTGAA | AGGGGTGAAGTTCCAGA | AGCCCCGTCCTCCTCTTC |
| AGCCCGG | GAGTGG | ACAGG |
| CTTAATCTAAGGGAAGT | GGGTTTGGCTGTTTGGG | ACCCTGTGAAGAGGAGG |
| TCCTGG | ATGGGG | ACGGGG |
| TCCGGTGGGCGAACTGA | GCCCTCGGGTCTCCCCG | TGAGGTAGGCATGTGCT |
| GCCAGG | GAGCGG | TTGTGG |
| GGACGCCCAGAGCGTGT | AATCAGAGTATTTCAAA | TGTCAGGACCCCAGAAA |
| CGCGGG | ACACGG | AGATGG |
| AATGCGCTTGCGCACTG | TCTAAAGCATTCTTTTAA | CCCGTTTCTACAAATAAT |
| ACTCGG | ATCGG | AGAGG |
| TTGAATGCCCGCCTCTTC | GCTTAAAGGAGAAACTC | TCAGGACCCCAGAAAAG |
| TACGG | TGAGGG | ATGGGG |
| ATGCGCTTGCGCACTGA | GGGGGAGGGGGCGGCA | GGATGAGGACGGAGGGC |
| CTCGGG | CTTCCGG | TGTGGG |
| GATTATAGCGATTATAT | GAATAGAAGCCCTGGGG | CAAGAGGAAGGGCTGCC |
| GAGCGG | TTGGGG | CTTTGG |
| CGGCGTGGAGGATCGGA | AGGGAACAGGGTATTGG | GCATGTGCTTTGTGGTCC |
| AAGAGG | AGGTGG | CCAGG |
| ATTATAGCGATTATATG | AGAACAGGAGAAACTGC | GTAGGAGAGTCCTGGGG |
| AGCGGG | ATGAGG | CCAGGG |
| CTCTTTCCGATCCTCCAC | GGGAACAGGGTATTGGA | GACCCTGTGAAGAGGAG |
| GCCGG | GGTGGG | GACGGG |
| AAAGCCCGGGCCGATTC | GCATGGTCTGAGCAGGC | CCTGAAGGGATGAGGAC |
| CGGTGG | AGCCGG | GGAGGG |
| GCACTTCCGGTCGGGCC | GGATCGGAAAGAGGAG | TAGAGGGCCTGCAGGTG |
| CTCGGG | GCGGTGG | GTGTGG |
| ATTCAAAGCGCAGGCGC | AGCAGGCAGCCGGATGC | CTTATCATTATGCTCATT |
| ACCTGG | TGCTGG | TTAGG |
| ATCCTCCACGCCGGCGC | CAGGAGGGGCTGAATGG | CCGTTTCTACAAATAAT |
| CTCGGG | GTCTGG | AGAGGG |
| GACTTCCGCTTCCGGGC | ATCCCTAGACCCCCAAC | TCAACACACACATATGT |
| GGGCGG | CCCAGG | GCAAGG |
| GGCACTTCCGGTCGGGC | GATGGAGGGGGCGGGGC | AGAGGAAGGGCTGCCCT |
| CCTCGG | CTAGGG | TTGGGG |
| AGACGGGAGACCCGGC | TTAAAAGAATGCTTTAG | ATCTCTCCCCATCTTTTC |
| GCCGGGG | AGGTGG | TGGGG |
| GACGCGAGCAGTGAAA | CACACTGAAGCCCCGCC | CATCTCTCCCCATCTTTT |
| GCCCGGG | CCCTGG | CTGGG |
| TCATATAATCGCTATAA | TCGGAAAGAGGAGGCGG | CATTTCCTCATCTACACC |
| TCCCGG | TGGCGG | TGGGG |
| GCGCGACTTCCGCTTCC | TGTGAGATGGGCGTGGT | CGTGGCCACACAGCTAG |
| GGGCGG | TCAGGG | AAGTGG |
| ATCACGCTGTGAGATGG | GCCCTGGGGTTGGGGGT | CCTTTGGGGTGGACAGG |
| GCGTGG | CTAGGG | CAGAGG |
| CTATTCCGCATGGTCTG | GCTTGGAGAGGGGGAAG | CCAGAGGCCCCCAGGAT |
| AGCAGG | AACAGG | GCTTGG |
| ACCATGCGGAATAGAAG | GCGGTGGCGGGGACCAG | CTTTGGGGTGGACAGGC |
| CCCTGG | GAGGGG | AGAGGG |
| CAAGCGCATTTCCCCCC | CGGTGGCGGGGACCAGG | CTCTCCTCCACACCACCT |
| TCCTGG | AGGGGG | GCAGG |
| GTGAAAGCCCGGGCCGA | GGAGGAGGCGCCGCCGC | AAAATGAGCATAATGAT |
| TTCCGG | TCCGGG | AAGGGG |
| ACGGGCGCGACTTCCGC | TGTTTGGGATGGGGCTG | CGGAGTGCTTACAGAGT |
| TTCCGG | AGTTGG | GCCAGG |
| CAGTGTGAGACCCGAGC | AAGGGGCGGGGCTTGGA | AAAACCTGAAGGGATGA |
| CGGAGG | TGGAGG | GGACGG |
| AGGGGGCGGCACTTCCG | GTTTGGGATGGGGCTGA | GCAGAGGGTCTGAGAGG |
| GTCGGG | GTTGGG | GTTTGG |
| ACCTGGCTCAGTTCGCC | GTGAAGGGGCGGGGCTT | GGGAGTTGAGAACACAG |
| CACCGG | GGATGG | AGAGGG |
| TTAAGCCACACCTCCGG | AAGCCCCGGCCCCCGGC | GGAAGGGCTGCCCTTTG |
| CTCGGG | CCTCGG | GGGTGG |
| TGAATGCCCGCCTCTTCT | AGAGAGTGGGAGCTTGG | CAAAGGGCAGCCCTTCC |
| ACGGG | AGAGGG | TCTTGG |
| GCCGCTCCGGGGAGACC | GTGGTTCAGGGCCAGGG | CCACACAGCTAGAAGTG |
| CGAGGG | GGCGGG | GCAGGG |
| GGTGCAACTTAAATGCT | CTTCCGGGCGGGCGGGC | TGGGAGTTGAGAACACA |
| ATGGGG | AGGCGG | GAGAGG |
| AGGTGCAACTTAAATGC | CCTCCTCGGCCTCCTCCC | AGTAGGAGAGTCCTGGG |
| TATGGG | GGCGG | GCCAGG |
| CGGGCGCGACTTCCGCT | ATGGGGCTGAGTTGGGA | CCCAGGTGTAGATGAGG |
| TCCGGG | GCCGGG | AAATGG |
| GGGAACCCCGCGACACG | AGGCGGTGGCGGGGACC | GTTGAGAACACAGAGAG |
| CTCTGG | AGGAGG | GGAAGG |
| GATCCTCCACGCCGGCG | GGAAAGAGGAGGCGGT | TGAATCCTCACAGTCCT |
| CCTCGG | GGCGGGG | GTGAGG |
| CGCGACTTCCGCTTCCG | CGGGCGGTGCGGGGCTG | AGACCCTGTGAAGAGGA |
| GGCGGG | CCGGGG | GGACGG |
| CATGCGGAATAGAAGCC | GGCGGTGGCGGGGACCA | GAGTATGGAGAGACAGC |
| CTGGGG | GGAGGG | AGAGGG |
| ACTTAAATGCTATGGGG | AGCCCTGGGGTTGGGGG | TCCATTTCCTCATCTACA |
| TGAAGG | TCTAGG | CCTGG |
| CGGTGGTTGTCAAGGAT | ATGGAGGCGATGGGGAG | GCCACACAGCTAGAAGT |
| AGTTGG | AGAAGG | GGCAGG |
| GAGGGGGCGGCACTTCC | TCCCTAGACCCCCAACC | AGCCCTTCCTCTTGGCTC |
| GGTCGG | CCAGGG | CCTGG |
| GAGGCGCCGGCGTGGAG | CAGAGAGTGGGAGCTTG | TGGGGTAGGGTGCAGGA |
| GATCGG | GAGAGG | GTATGG |
| GGGAGACCCGAGGGCCC | AGGGGCGGGGCTTGGAT | GAGGACGGAGGGCTGTG |
| GACCGG | GGAGGG | GGTGGG |
| ACTCTGATTCTGCTGCAT | CGGTGCGGGGCTGCCGG | GAGACAGCAGAGGGTCT |
| GCTGG | GGAGGG | GAGAGG |
| GTCGGGCCCTCGGGTCT | CGGGGCTTGGATGGAGG | GGCCAGGGAGCCAAGAG |
| CCCCGG | GGGCGG | GAAGGG |
| CGGAATAGAAGCCCTGG | GCGGTGCGGGGCTGCCG | GGAGTATGGAGAGACAG |
| GGTTGG | GGGAGG | CAGAGG |
| CTTATAATCACGCTGTG | GATGGGGCTGAGTTGGG | TGAGGACGGAGGGCTGT |
| AGATGG | AGCCGG | GGGTGG |
| GAGAAACTGCATGAGGG | CTCTCCAAGCTCCCACTC | CTCTGCCTGTCCACCCCA |
| CGAGGG | TCTGG | AAGGG |
| AAAGCATTCTTTTAAAT | GCGGGCGGTGCGGGGCT | GGGATGAGGACGGAGG |
| CGGTGG | GCCGGG | GCTGTGG |
| CTCGGGTCTCCCCGGAG | GAGAGTGGGAGCTTGGA | GCCTACCTCACAGGACT |
| CGGCGG | GAGGGG | GTGAGG |
| GGGGACGAAGACAGAC | TGCGGGGCTGCCGGGGA | GGAAAGAAAACTGAGTC |
| TTTGAGG | GGGGGG | CAGAGG |
| TGAGACCCGAGCCGGAG | GGGCTTGGATGGAGGGG | GGGCCAGGGAGCCAAGA |
| GTGTGG | GCGGGG | GGAAGG |
| GGACTTAGAAGGTGCTG | GGTGGCGGGGACCAGGA | CTGTGGGTGGGGTAGGG |
| ATGGGG | GGGGGG | TGCAGG |
| CGCGCCCGTTCCCGAGG | AGAGTGGGAGCTTGGAG | GGGTGGACAGGCAGAGG |
| CGCCGG | AGGGGG | GACTGG |
| GAGACGGGAGACCCGG | TTCCGGGCGGGCGGGCA | CTAAAATGAGCATAATG |
| CGCCGGG | GGCGGG | ATAAGG |
| CGTGGAGGATCGGAAAG | GGGCGGGGCTTGGATGG | GGAGGACGGGGCTGAGG |
| AGGAGG | AGGGGG | ACACGG |
| TTAATCTAAGGGAAGTT | AGGAGGCGGTGGCGGGG | AGACAGCAGAGGGTCTG |
| CCTGGG | ACCAGG | AGAGGG |
| CTTAGCAGGAGGGGCTG | GGGGGGGGCTTGGATG | TAAAATGAGCATAATGA |
| AATGGG | GAGGGG | TAAGGG |
| GAAGGAAGTACTTAATC | TGGGGTGAAGGGGCGGG | ATTTTAGGATAAAACCT |
| TAAGGG | GCTTGG | GAAGGG |
| TGTCAAGTGTGGTAAAT | GGCGGGCGGTGCGGGGC | ACTTCTAGCTGTGTGGCC |
| CCATGG | TGCCGG | ACGGG |
| TTCCCGAGGCGCCGGCG | GGTTGTCAAGGATAGTT | ATTAGAGAAGGAAAAGA |
| TGGAGG | GGGAGG | GGTGGG |
| GCTTAGCAGGAGGGGCT | GGGGCTTGGATGGAGGG | CTGCAGGTGGTGTGGAG |
| GAATGG | GGCGGG | GAGAGG |
| TGCTATGGGGTGAAGGG | GCGGGCAGGCGGGCGGT | AGGACGGAGGGCTGTGG |
| GCGGGG | GCGGGG | GTGGGG |
| GGGGTGAAGTTCCAGAG | GTGCGGGGCTGCCGGGG | AGGGCCTGCAGGTGGTG |
| AGTGGG | AGGGGG | TGGAGG |
| CGTGGTTCAGGGCCGAG | GGTGCGGGGCTGCCGGG | TGCAGGTGGTGTGGAGG |
| GGCCGG | GAGGGG | AGAGGG |
| CGGGGTTCCCGTAGAAG | CCGCCTCCTCGGCCTCCT | CCAGAAAAGATGGGGAG |
| AGGCGG | CCCGG | AGATGG |
| GAGGAGGCGCCGCCGCT | CGGCGCCTCCTCCGCCTC | AGGTGTAGATGAGGAAA |
| CCGGGG | CTCGG | TGGAGG |
| ACTCTGAGGGAACAGGG | GGATGGAGGGGGCGGG | AACACAGAGAGGGAAG |
| TATTGG | GCCTAGG | GTGAAGG |
| CTTAAATGCTATGGGGT | TATTGGAGGTGGGACTT | GAGGGAAGGTGAAGGTG |
| GAAGGG | AGAAGG | AGTAGG |
| TTAAATGCTATGGGGTG | AGGGGGCGGGGCCTAGG | GTGAGCCAGCGCGCTGG |
| AAGGGG | GAGGGG | CCTGGG |
| CTCCTTTAAGCCCCGGC | GAGGGGGCGGGGCCTAG | AATTAGAGAAGGAAAAG |
| CCCCGG | GGAGGG | AGGTGG |
| GGCCGGGGGCCGGGGCT | AGGGCCGAGGGCCGGGG | AAAAGAGGTGGGGAGTG |
| TAAAGG | GCCGGG | GGTGGG |
| TGGGCGTGGTTCAGGGC | GGCGGGCAGGCGGGCGG | TGTGAGCCAGCGCGCTG |
| CGAGGG | TGCGGG | GCCTGG |
| GGAGGCACTCAAAAGTC | CAGGGCCGAGGGCCGGG | TGAAGAGGAGGACGGG |
| ACGAGG | GGCCGG | GCTGAGG |
| GAGGTGCAACTTAAATG | GGCTGCCGGGGAGGGGG | CGGAGGGCTGTGGGTGG |
| CTATGG | GAGGGG | GGTAGG |
| GGGACTTAGAAGGTGCT | GGGCGGGCAGGCGGGCG | CACTTCTAGCTGTGTGGC |
| GATGGG | GTGCGG | CACGG |
| CTTCAGTGTGAGACCCG | GCCGCCCCCTCCCCCCTC | AAGGAAAAGAGGTGGG |
| AGCCGG | CCCGG | GAGTGGG |
| GGAGAAACTGCATGAGG | GGAGGGGGCGGGGCCTA | TTAGAGAAGGAAAAGAG |
| GCGAGG | GGGAGG | GTGGGG |
| TGGGAGAGTGGAAGCTT | GCTGCCGGGGAGGGGGG | GGAGGGCTGTGGGTGGG |
| AGCAGG | AGGGGG | GTAGGG |
| GGAGACGGGAGACCCG | GGGCTGCCGGGGAGGGG | AAAAGATGGGGAGAGAT |
| GCGCCGG | GGAGGG | GGAAGG |
| GGGCTGAATGGGTCTGG | GGAGGATCGGAAAGAG | GAGATGGAAGGAATTAG |
| GTTTGG | GAGGCGG | AGAAGG |
| AGACAGACTTTGAGGCA | GGGGCTGCCGGGGAGGG | AGGAATTAGAGAAGGAA |
| GTAGGG | GGGAGG | AAGAGG |
| TGGTTCAGGGCCGAGGG | GGAGGAGGCCGAGGAG | GGATAAAACCTGAAGGG |
| CCGGGG | GCGGAGG | ATGAGG |
| GAGAGTGGAAGCTTAGC | GCCGGGGAGGGGGGAG | GAAAAGAGGTGGGGAGT |
| AGGAGG | GGGGCGG | GGGTGG |
| CGCCGCTCCGGGGAGAC | GGAGGCCGAGGAGGCG | TCCTCACAGTCCTGTGA |
| CCGAGG | GAGGAGG | GGTAGG |
| AGAAGGAAGTACTTAAT | CACTCCGTACATAGAAG | TCTGTAATCACCCAGCA |
| CTAAGG | GTATGG | CTTTGG |
| ATGCTATGGGGTGAAGG | CCGCACAAGGATGGATG | AAGGTATGGACTGGGCA |
| GGCGGG | AGTGGG | CAGTGG |
| CACGACTGCTATGGAGG | TATCAGCGTAATTAGGA | GAAGGAAAAGAGGTGG |
| CGATGG | GGGAGG | GGAGTGG |
| GGCTTAAAGGAGAAACT | TTTCTATCAGCGTAATTA | AAAAGGAGAGAAAGTA |
| CTGAGG | GGAGG | GAGACGG |
| CTGAGGGAACAGGGTAT | CGTACATAGAAGGTATG | ACAGATGTGAGCCAGCG |
| TGGAGG | GACTGG | CGCTGG |
| ATGGGCGTGGTTCAGGG | GGCTCTTAGGGGGCGGT | CTGTAATCACCCAGCAC |
| CCAGGG | TCCTGG | TTTGGG |
| TGCTAAGCTTCCACTCTC | TCCGCACAAGGATGGAT | CCTGGGGCCAGGGAGCC |
| CCAGG | GAGTGG | AAGAGG |
| TCTCCGCCGGGAGGAGG | ACAAATAATAGAGGGCC | TAATCACCCAGCACTTT |
| CCGAGG | TGCAGG | GGGAGG |
| GACTTAGAAGGTGCTGA | CAGTCCATACCTTCTATG | CTCTATTATTTGTAGAAA |
| TGGGGG | TACGG | CGGGG |
| AAGACAGACTTTGAGGC | CCCCTCTACAAAGCTGT | GAGGTTCAGTCACTTGC |
| AGTAGG | GTCAGG | CCGTGG |
| TTTTAAATCGGTGGTTGT | TTCTATCAGCGTAATTAG | GGGAGGTGGAGGCTGGA |
| CAAGG | GAGGG | GGATGG |
| AAGAATGCTTTAGAGGT | GTAAGCACTCCGTACAT | TCACCCAGCACTTTGGG |
| GGGAGG | AGAAGG | AGGTGG |
| GATGGGCGTGGTTCAGG | CAGTTTCTATCAGCGTA | CAGCTGTCATCCCAGCA |
| GCCAGG | ATTAGG | CTTTGG |
| CCGGCGGAGACCCCGGC | CAATTGTCCAAGCATCC | GAGGAAATGGAGGCACA |
| GCCGGG | TGGGGG | GAGAGG |
| CCTCCTCCCGGCGGAGA | GTCCTGACACAGCTTTGT | GCCTCCACCTCCCAAAG |
| CCCCGG | AGAGG | TGCTGG |
| GGGGTTCCCGTAGAAGA | ATATGTGCAAGGCTCTT | GGGGTTTTGCTTTGTTGC |
| GGCGGG | AGGGGG | CCAGG |
| GTTGGGAGGCCAGCAGC | ACATATGTGCAAGGCTC | CTGGAGGATGGCGTGAG |
| ATCCGG | TTAGGG | TCCAGG |
| TGGGACTTAGAAGGTGC | CTCCAATTGTCCAAGCA | CCCAGCACTTTGGGAGG |
| TGATGG | TCCTGG | TGGAGG |
| AATGCTATGGGGTGAAG | CACATATGTGCAAGGCT | AGCTGTCATCCCAGCAC |
| GGGCGG | CTTAGG | TTTGGG |
| CGGGTGGGAGACGGGA | GCCTAAATCTGGAAGGC | TTTTGCTTTGTTGCCCAG |
| GACCCGG | TAGGGG | GCTGG |
| TGGGCGTGGTTCAGGGC | GCAGGGCTTACATTTGA | CAGGAGTTTCAGACCAG |
| CAGGGG | TTCTGG | CCTGGG |
| GTGGTTCAGGGCCGAGG | AATAATAGAGGGCCTGC | GCAGGAGAGCTGCTTGA |
| GCCGGG | AGGTGG | GCCCGG |
| AGAGTGGAAGCTTAGCA | CATATGTGCAAGGCTCT | CCAGGAGTTTCAGACCA |
| GGAGGG | TAGGGG | GCCTGG |
| GGTTCAGGGCCGAGGGC | GCTAGGGGGTGATCCGC | TGTCATCCCAGCACTTTG |
| CGGGGG | ACAAGG | GGAGG |
| AGGAGAAACTCTGAGGG | AGCCTAAATCTGGAAGG | GCACTTTGGGAGGTGGA |
| AACAGG | CTAGGG | GGCTGG |
| GAACAGGAGAAACTGC | TGAGGACACGGCCGCGG | CTTTGGGAGGTGGAGGC |
| ATGAGGG | CGCTGG | TGGAGG |
| TGGGTTTGGCTGTTTGG | TGCGGGGAGACCCAGCG | GCCTCAGCCTCCCAAAG |
| GATGGG | CCGCGG | TGCTGG |
| ACAGACTTTGAGGCAGT | GTCAGGACCCCAGAAAA | CCTCAGCCTCCCAAAGT |
| AGGGGG | GATGGG | GCTGGG |
| TGAGCGGGCAGTGTCAA | TAATTAGGAGGGAGGAC | GCACTTTGGGAGGCTGA |
| GTGTGG | TCTGGG | GGCAGG |
| GGTGGTTGTCAAGGATA | CCTAAATCTGGAAGGCT | |
| GTTGGG | AGGGGG | |
| CCCGGCGGAGACCCCGG | ACTGGAAGCCTAAATCT | |
| CGCCGG | GGAAGG | |

In some aspects, the ZNF865-specific gRNA is GTCAGGACCCCAGAAAAGAT (SEQ ID NO: 15), GCGCACAAGGATGGATGAGT (SEQ ID NO:16), GGGGACTGGAAGCCTAAATC (SEQ ID NO: 17), GGGGGTGATCCGCACAAGGA (SEQ ID NO:18), or GTCTGCCTGTCCACCCCAAA (SEQ ID NO:19). In some aspects, the ZNF865-specific gRNAs can be optimized. For example, GACGCCCAGAGCGTGTCGCG (SEQ ID NO:21), GAGGCGGGCATTCAAAGCGC (SEQ ID NO:22), TCGCCCACCGGAATCGGCCC (SEQ ID NO:23), atcctccacgccggcgcctc (SEQ ID NO:24), acttccgcttccgggcgggc (SEQ ID NO:25), or gcACTTCCGGTCGGGCCCTC (SEQ ID NO:26) can be optimized. Optimization can refer to the ability to titre the degree to which the expression of ZNF865 can be regulated. For example, optimization can refer to increasing expression by as much as possible or any level below that with specific gRNA design. Changing the gRNA sequence can allow for specific control the degree of expression of ZNF865.

In some aspects, the ZNF865-specific gRNA can bind to a target site of the ZNF865 gene. In some aspects, the target site is within a coding region of the ZNF865 gene. In some aspects, the target site is within a non-coding region of the ZNF865 gene. In some aspects, the target site is upstream of the ZNF865 gene.

In some aspects, the vector comprising the ZNF865-specific gRNA can also comprise a nucleic acid sequence capable of encoding a Cas protein or a nucleic acid sequence capable of encoding a Cas protein and a nucleic acid sequence capable of encoding a transcriptional activator. Vectors comprising all of these elements allow the transcriptional activator to boost expression of ZNF865 once the ZNF865-specific gRNA directs the components to the ZNF865 gene.

### 4. Other Vector Features

In some aspects, any of the disclosed vectors can comprise any of the following vector features.

There are a number of compositions and methods which can be used to deliver the disclosed nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

Expression vectors can be any nucleotide construction used to deliver genes or gene fragments into cells (e.g., a plasmid), or as part of a general strategy to deliver genes or gene fragments, e.g., as part of recombinant retrovirus or adenovirus (Ram et al. Cancer Res. 53:83-88, (1993)). For example, disclosed herein are expression vectors comprising a nucleic acid sequence capable of encoding a polypeptide comprising two heterologous polypeptide domains, wherein the first polypeptide domain comprises a Cas protein and wherein the second polypeptide domain comprises ZNF865 or fragment thereof.

The “control elements” present in an expression vector are those non-translated regions of the vector-enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the pBLUESCRIPT phagemid (Stratagene, La Jolla, Calif.) or pSPORT1 plasmid (Gibco BRL, Gaithersburg, Md.) and the like may be used. If it is necessary to generate a cell line that contains multiple copies of the sequence encoding a polypeptide, vectors based on SV40 or EBV may be advantageously used with an appropriate selectable marker.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osbome, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

In some aspects, a promoter can be regulatable. The promoter or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

Optionally, the promoter or enhancer region can act as a constitutive promoter or enhancer to maximize expression of the polynucleotides of the invention. In certain constructs the promoter or enhancer region can be active in all eukaryotic cell types, even if it is only expressed in a particular type of cell at a particular time.

In some aspects, a vector comprises one or more pol promoters, one or more pol promoters II, one or more pol III promoters, or combinations thereof. Examples of pol II promoters include, but are not limited to the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phospho glycerol kinase (PGK) promoter, and the EF1α promoter. In some aspects, pol II promoters can be engineered to confer tissue specific and inducible regulation of gRNAs. Examples of pol III promoters include, but are not limited to, U6 and H1 promoters. In an aspect, the promoter is U6. In an aspect, the promoter operably linked to the gRNA is a Pol III promoter, human u6, mouse U6, H1, or 7SK. Examples of promoters can be those derived from polyoma, adenovirus 2, cytomegalovirus, simian virus 40, and others disclosed herein and known in the art.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases.

The expression vectors can include a nucleic acid sequence encoding a marker product. This marker product can be used to determine if the gene has been delivered to the cell and once delivered is being expressed. Marker genes can include, but are not limited to the E. coli lacZ gene, which encodes β-galactosidase, and the gene encoding the green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

Another type of selection that can be used with the composition and methods disclosed herein is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P. J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

As used herein, plasmid or viral vectors are agents that transport the disclosed nucleic acids, such as a nucleic acid sequence capable of encoding one or more of the disclosed peptides into the cell without degradation and include a promoter yielding expression of the gene in the cells into which it is delivered. In some embodiments the nucleic acid sequences disclosed herein are derived from either a virus or a retrovirus. Viral vectors are, for example, Adenovirus, Adeno-associated virus, Herpes virus, Vaccinia virus, Polio virus, lentivirus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction abilities (i.e., ability to introduce genes) than chemical or physical methods of introducing genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

Retroviral vectors, in general, are described by Verma, I. M., Retroviral vectors for gene transfer. In Microbiology, Amer. Soc. for Microbiology, pp. 229-232, Washington, (1985), which is hereby incorporated by reference in its entirety. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference in their entirety for their teaching of methods for using retroviral vectors for gene therapy.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serves as the priming site for the synthesis of the second strand of DNA synthesis, and specific sequences near the ends of the LTRs that enable the insertion of the DNA state of the retrovirus to insert into the host genome. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)) the teachings of which are incorporated herein by reference in their entirety for their teaching of methods for using retroviral vectors for gene therapy. Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol., 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. Optionally, both the E1 and E3 genes are removed from the adenovirus genome.

Another type of viral vector that can be used to introduce the polynucleotides of the invention into a cell is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, CA, which can contain the herpes simplex virus thymidine kinase gene, HSV-tk, or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus. Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference in its entirety for material related to the AAV vector.

The inserted genes in viral and retroviral vectors usually contain promoters, or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Other useful systems include, for example, replicating and host-restricted non-replicating vaccinia virus vectors. In addition, the disclosed nucleic acid sequences can be delivered to a target cell in a non-nucleic acid based system. For example, the disclosed polynucleotides can be delivered through electroporation, or through lipofection, or through calcium phosphate precipitation. The delivery mechanism chosen will depend in part on the type of cell targeted and whether the delivery is occurring for example in vivo or in vitro.

### E. Compositions

Disclosed are compositions comprising the disclosed polypeptides, nucleic acid constructs or vectors. Disclosed are compositions comprising a nucleic acid construct, wherein the nucleic acid construct comprises a polynucleotide capable of encoding a polypeptide comprising two heterologous polypeptide domains, wherein the first polypeptide domain comprises a Cas protein and wherein the second polypeptide domain comprises ZNF865 or fragment thereof. Also disclosed are compositions comprising a vector, such as a viral vector, comprising a nucleic acid construct, wherein the nucleic acid construct comprises a nucleic acid sequence that encodes only ZNF865, a fragment of ZNF865, a variant of ZNF865, any of the polypeptides disclosed herein, or a ZNF865-specific gRNA.

The disclosed compositions can further comprise a pharmaceutically acceptable carrier.

### 1. Delivery of Compositions

In the methods described herein, delivery (or administration) of the compositions to cells can be via a variety of mechanisms. As defined above, disclosed herein are compositions comprising any one or more of the polypeptides, nucleic acids, and/or vectors described herein can be used to produce a composition which can also include a carrier such as a pharmaceutically acceptable carrier. For example, disclosed are pharmaceutical compositions, comprising the peptides disclosed herein, and a pharmaceutically acceptable carrier.

For example, the compositions described herein can comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, PA 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

The disclosed delivery techniques can be used not only for the disclosed compositions but also the disclosed nucleic acid constructs and vectors.

### F. Recombinant Cells

Disclosed are recombinant cells comprising one or more of the disclosed nucleic acid constructs or vectors. For example, disclosed are recombinant cells comprising a nucleic acid construct, wherein the nucleic acid construct is a polynucleotide capable of encoding a polypeptide comprising two heterologous polypeptide domains, wherein the first polypeptide domain comprises a Cas protein and wherein the second polypeptide domain comprises ZNF865, a fragment of ZNF865, or a variant of ZNF865.

The compositions and methods disclosed herein can be performed on any cell type. In some aspects, the cell is a eukaryotic cell. In some aspects, the eukaryotic cell is a mammalian cell. In some aspects, the mammalian cell is a human cell. For example, the cell can be a stem cell, bone cell, blood cell, muscle cell, fat cell, skin cell, nerve cell, endothelial cell. In some aspects, the cell is a T cell (e.g., CD4+ T cell, CD8+ T cell).

### G. CRISPR-Cas System

In some aspects, the disclosed methods use a CRISPR (clustered regularly interspersed short palindromic repeats) or CRISPR-Cas system. As used herein, “CRISPR system” or “CRISPR-Cas system” refers to transcripts and other elements involved in the expression of or directing the activity of CRISPR-associated (“Cas”) genes, including sequences encoding a Cas gene, a guide sequence (also referred to as a “spacer” in the context of an endogenous CRISPR system; e.g. guide RNA or gRNA), or other sequences and transcripts from a CRISPR locus.

### 1. CRISPR-Cas Systems to Upregulate ZNF865

Disclosed are CRISPR-Cas systems designed to boost expression of the target gene, ZNF865. Thus, disclosed are CRISPR-Cas systems comprising one or more vectors comprising one or more nucleotide sequences encoding a CRISPR-Cas system guide RNA (gRNA), wherein the gRNA hybridizes with a target sequence of ZNF865 in a cell; and a nucleotide sequence encoding a deactivated Cas (dCas) protein fused to a transcriptional activator, wherein the nucleotide sequences for the gRNA and the deactivated Cas protein fused to a transcriptional activator are located on the same or different vectors of the same system, wherein the gRNA targets and hybridizes with the target sequence and directs the deactivated Cas protein fused to a transcriptional activator to the ZNF865.

In some aspects, the gRNA sequence is one or more of the sequences in Table 1.

In some aspects, the target sequence of ZNF865 is a nucleic acid sequence upstream of a transcriptional start site of ZNF865. In some aspects, the target sequence of ZNF865 is a nucleic acid sequence in the promoter of ZNF865. In some aspects, the target sequence of ZNF865 is a nucleic acid sequence in the coding or non-coding region of ZNF865.

In some aspects, the dCas protein is fused to a transcriptional activator. The transcriptional activator serves to boost expression of the target gene (e.g. ZNF865). In some aspects, the transcriptional activator can be, but is not limited to, VPR, VP64, SAM, CNP, SPH, SunTag, or p300. Any known transcriptional activator can be used.

In some aspects, one or more of the nucleotide sequences can be operably linked to a promoter. In some aspects, a promoter is operably linked to the one or more nucleotide sequences encoding a CRISPR-Cas system gRNA.

In some aspects, a regulatory element is operably linked to the nucleotide sequence encoding a deactivated Cas protein fused to a transcriptional activator. In some aspects, the regulatory element can be a promoter, promoter enhancer, internal ribosomal entry site (IRES) or other element that are capable of controlling expression (e.g., transcription termination signals, including but not limited to polyadenylation signals and poly-U sequences).

In some aspects, the gRNA hybridizes with a target sequence of ZNF865 in a cell. In some aspects, the cell is a eukaryotic cell. In some aspects, the eukaryotic cell is a mammalian cell. In some aspects, the mammalian cell is a human cell. For example, the cell can be a stem cell, bone cell, blood cell, muscle cell, fat cell, skin cell, nerve cell, endothelial cell. In some aspects, the cell is a T cell (e.g., CD4+ T cell, CD8+ T cell).

In some aspects, the system is packaged into a single lentiviral, adenoviral or adeno-associated virus particle.

### 2. CRISPR-Cas Systems Using ZNF865 to Upregulate a Target Gene

Disclosed are CRISPR-Cas systems designed to boost expression of a target gene by using dCas fused to ZNF865 as a transcriptional activator. Disclosed are CRISPR-Cas systems comprising one or more vectors comprising one or more nucleotide sequences encoding a CRISPR-Cas system guide RNA (gRNA), wherein the gRNA hybridizes with a target sequence of a DNA locus in a cell; and a nucleotide sequence encoding a deactivated Cas protein fused to ZNF865, wherein the nucleotides encoding the gRNA and the dCas protein fused to ZNF865 are located on the same or different vectors of the same system, wherein the gRNA targets and hybridizes with the target sequence and directs the deactivated Cas protein fused to ZNF865 to the DNA locus.

In some aspects, the DNA locus is a target gene (e.g. gene of interest). In some aspects, the target sequence that hybridizes with the gRNA is a nucleic acid sequence upstream of a transcriptional start site of a target gene. In some aspects, the target gene can be Aggrecan or Collagen II. Thus, in some aspects the target sequence that hybridizes with the gRNA is a nucleic acid sequence upstream of a transcriptional start site of Aggrecan or Collagen II.

In some aspects, the gRNA sequence can be a sequence that hybridizes to a target sequence of Aggrecan or Collagen II. Examples of these gRNA sequences can be found in U.S. patent application Ser. No. 17/284,908 which is hereby incorporated by reference for its teaching of gRNA sequences that hybridizes to a target sequence of Aggrecan or Collagen II.

In some aspects, one or more of the nucleotide sequences can be operably linked to a promoter. In some aspects, a promoter is operably linked to the one or more nucleotide sequences encoding a CRISPR-Cas system gRNA.

In some aspects, a regulatory element is operably linked to the nucleotide sequence encoding a deactivated Cas protein fused to ZNF865, a fragment of ZNF865, or a variant of ZNF865. As used herein, the term “regulatory element” refers to promoters, promoter enhancers, internal ribosomal entry sites (IRES) and other elements that are capable of controlling expression (e.g., transcription termination signals, including but not limited to polyadenylation signals and poly-U sequences). Regulatory elements can direct constitutive expression. Regulatory elements can be tissue-specific. Examples of tissue-specific promoters can direct expression in a desired tissue of interest (e.g., muscle, neuron, bone, skin, blood, intervertebral disc), specific organs (e.g., liver, pancreas, brain, spinal cord), or particular cell types (peripheral nerves, annulus fibrosus, nucleus pulposus, chondrocytes). Regulatory elements can also direct expression in a temporal-dependent manner including but not limited to cell-cycle dependent or developmental stage-dependent. Temporal-dependent expression can be tissue or cell-type specific. Regulatory element can also refer to enhancer elements. Examples of enhancer elements include but are not limited to WPRE, CMV enhancers, and SV40 enhancers. In an aspect, the regulatory element is hUbC. In an aspect, the hUbC promoter is operably linked to a nucleotide sequence encoding a RNA-directed nuclease. Generally, any constitutive promoter can be operably linked to a nucleotide sequence encoding a RNA-directed nuclease. Specific gene specific promoters can be used. Such promoters allow cell specific expression or expression tied to specific pathways. Any promoter that is active in mammalian cells can be used. In an aspect, the promoter is an inducible promoter including, but not limited to, Tet-on and Tet-off systems. Such inducible promoters can be used to control the timing of the desired expression

Examples of regulatory elements include, but are not limited to hUBC and ef-1alpha.

In some aspects, the gRNA hybridizes with a target sequence of a target gene in a cell. In some aspects, the cell is a eukaryotic cell. In some aspects, the eukaryotic cell is a mammalian cell. In some aspects, the mammalian cell is a human cell. For example, the cell can be a stem cell, bone cell, blood cell, muscle cell, fat cell, skin cell, nerve cell, endothelial cell. In some aspects, the cell is a T cell (e.g., CD4+ T cell, CD8+ T cell).

In some aspects, the system is packaged into a single lentiviral, adenoviral or adeno-associated virus particle.

### H. Methods of Increasing ZNF865

In some aspects, increasing ZNF865 can have positive downstream effect in a cell. In some aspects, ZNF865 acts as a transcription activator.

Disclosed are methods of increasing ZNF865 in a cell using gene therapy, protein therapy, or CRISPR therapy.

Disclosed are methods of increasing ZNF865 in a cell comprising contacting a cell with a vector comprising a nucleic acid sequence encoding a ZNF865 protein (gene therapy); a recombinant ZNF865 protein or fragment thereof (protein therapy); or one or more vectors comprising one or more nucleic acid sequences encoding a ZNF865-specific guide RNA (gRNA) and a dCas9 fused to a transcriptional activator, wherein the transcriptional activator upregulates ZNF865 (CRISPR therapy).

In some aspects, the method of increasing ZNF865 in a cell comprising contacting a cell with a vector comprising a nucleic acid sequence encoding a ZNF865 protein comprises using one or more of the vectors comprising a ZNF865 nucleic acid sequence described herein.

In some aspects, the method of increasing ZNF865 in a cell comprising contacting a cell with a recombinant ZNF865 protein comprises using any of the ZNF865 proteins or fragments thereof described herein. For example, any of SEQ ID NOs:1-3 or variants thereof can be used.

In some aspects, the method of increasing ZNF865 in a cell comprising contacting a cell with one or more vectors comprises using one or more of the nucleic acid sequences encoding a ZNF865-specific gRNA and a dCas9 fused to a transcriptional activator, wherein the transcriptional activator upregulates ZNF865 described herein. In some aspects, only the ZNF865-specific gRNA is delivered using a vector while the dCas9 fused to a transcriptional activator can be delivered as a protein.

In some aspects, the method of increasing ZNF865 in a cell can be performed in vitro or in vivo. In some aspects, the method is performed in vivo. In some aspects, contacting a cell comprises administering to a subject comprising the cell a vector comprising a nucleic acid sequence encoding a ZNF865 protein; a recombinant ZNF865 protein; or one or more vectors comprising one or more nucleic acid sequences encoding a ZNF865-specific guide RNA (gRNA) and a dCas9 fused to a transcriptional activator, wherein the transcriptional activator upregulates ZNF865. In some aspects, only the ZNF865-specific gRNA is delivered using a vector while the dCas9 fused to a transcriptional activator can be delivered as a protein.

In some aspects, the gRNA hybridizes with a target sequence of ZNF865 in the cell. In some aspects, the ZNF865-specific gRNA can be any of those listed in Table 1.

In some aspects, the target sequence is a nucleic acid sequence upstream of a transcriptional start site of ZNF865.

In some aspects, the transcriptional activator can be, but is not limited to, VPR, VP64, SAM, CNP, SPH, SunTag, or p300.

In some aspects, the vectors can be one or more of those vectors described herein. In some aspects, the vectors can comprise any of the features described herein. For example, in some aspects, a promoter is operably linked to the nucleotide sequence encoding a ZNF865-specific gRNA. In some aspects, a regulatory element is operably linked to the nucleotide sequence encoding a dCas9 fused to a transcriptional activator. In some aspects, the regulatory element can be a promoter, promoter enhancer, internal ribosomal entry site (IRES) or other element that are capable of controlling expression (e.g., transcription termination signals, including but not limited to polyadenylation signals and poly-U sequences).

In some aspects, the cell is a eukaryotic cell. In some aspects, the eukaryotic cell is a mammalian cell. In some aspects, the mammalian cell is a human cell. For example, the cell can be a stem cell, bone cell, blood cell, muscle cell, fat cell, skin cell, nerve cell, endothelial cell. In some aspects, the cell is a T cell (e.g., CD4+ T cell, CD8+ T cell). Thus, in some aspects, ZNF865 can be upregulated in a T cell causes activation of the T cell which leads to T cell proliferation.

In some aspects, one or more vectors are lentiviral, adenoviral, or adeno-associated virus particles.

### I. Methods of Decreasing ZNF865

Disclosed are methods of decreasing ZNF865 in a cell. In some aspects, decreasing ZNF865 can be a complete knockout or a knockdown of ZNF865.

In some aspects, the methods of decreasing ZNF865 in a cell comprise contacting a cell with a sufficient amount of siRNA or shRNA, wherein the siRNA or shRNA binds to the ZNF865 gene.

In some aspects, decreasing ZNF865 can be achieved using a CRISPR knock down system. Disclosed are methods of decreasing ZNF865 in a cell comprising contacting a cell with one or more vectors comprising a nucleic acid sequence encoding a ZNF865-specific gRNA and a Cas9. In some aspects, a nucleic acid sequence encoding a ZNF865-specific gRNA can be any of those sequences of Table 1.

In some aspects, the methods of decreasing ZNF865 can lead to cell death. Thus, disclosed are methods of inducing cell death comprising decreasing expression of ZNF865. In some aspects, the cell death can be cancer cell death.

Disclosed are methods of treating a subject having cancer comprising administering to the subject a composition capable of knocking down ZNF865. In some aspects, the composition can comprise siRNA or shRNA, wherein the siRNA or shRNA binds to a ZNF865 gene. In some aspects, the composition can comprise one or more vectors comprising a nucleic acid sequence encoding a ZNF865-specific gRNA and a Cas9. In some aspects, a nucleic acid sequence encoding a ZNF865-specific gRNA can be any of those sequences of Table 1.

### J. Methods of Upregulating a Target Using ZNF865

In some aspects, ZNF865 can be used to increase transcription of a target gene.

Disclosed are methods of upregulating expression of a target gene by using ZNF865 as a transcriptional activator in a cell using gene therapy, protein therapy, or CRISPR therapy. In some aspects, the methods can be performed in vitro or in vivo.

Disclosed are methods of upregulating expression of a target gene comprising contacting a cell with a therapeutically effective amount of a vector comprising a nucleic acid sequence encoding a ZNF865 protein, a fragment of ZNF865, or a variant of ZNF865; a therapeutically effective amount of a ZNF865 protein; or one or more vectors comprising one or more nucleic acid sequences encoding a target gene-specific guide RNA (gRNA) and a dCas9 fused to ZNF865, wherein ZNF865 protein, a fragment of ZNF865, or a variant of ZNF865 upregulates the target gene.

In some aspects, the method of upregulating expression of a target gene comprising contacting a cell with a therapeutically effective amount of a vector comprising a nucleic acid sequence encoding a ZNF865 protein, a fragment of ZNF865, or a variant of ZNF865 protein comprises using one or more of the vectors comprising a ZNF865 nucleic acid sequence described herein.

In some aspects, the method of upregulating expression of a target gene in a cell comprising contacting a cell with a therapeutically effective amount of a ZNF865 protein comprises using any of the ZNF865 proteins, variants or fragments thereof described herein. For example, any of SEQ ID NOs:1-3 or variants thereof can be used.

In some aspects, the method of upregulating expression of a target gene in a cell comprising contacting a cell with one or more vectors comprising one or more nucleic acid sequences encoding a target gene-specific gRNA and a dCas9 fused to ZNF865, a fragment of ZNF865, or a variant of ZNF865. In some aspects, the ZNF865 upregulates the target gene. In some aspects, only the target gene-specific gRNA is delivered using a vector while the dCas9 fused to ZNF865, a fragment of ZNF865, or a variant of ZNF865 can be delivered as a protein.

In some aspects, the target gene can be aggrecan or collagen II. Thus, in some aspects the target sequence that hybridizes with the gRNA is a nucleic acid sequence upstream of a transcriptional start site of aggrecan or collagen II.

In some aspects, the target gene-specific gRNA sequence can be a sequence that hybridizes to a target sequence of a target gene, such as, but not limited to, aggrecan or collagen II. Examples of these gRNA sequences can be found in U.S. patent application Ser. No. 17/284,908 which is hereby incorporated by reference for its teaching of gRNA sequences that hybridizes to a target sequence of Aggrecan or Collagen II.

In some aspects, the upregulation of the target gene can be a direct regulation from the ZNF865 protein, fragment of ZNF865, or variant of ZNF865. In some aspects, the upregulation of the target gene can be an indirect regulation through other gene changes initiated by the ZNF865 protein, fragment of ZNF865, or variant of ZNF865.

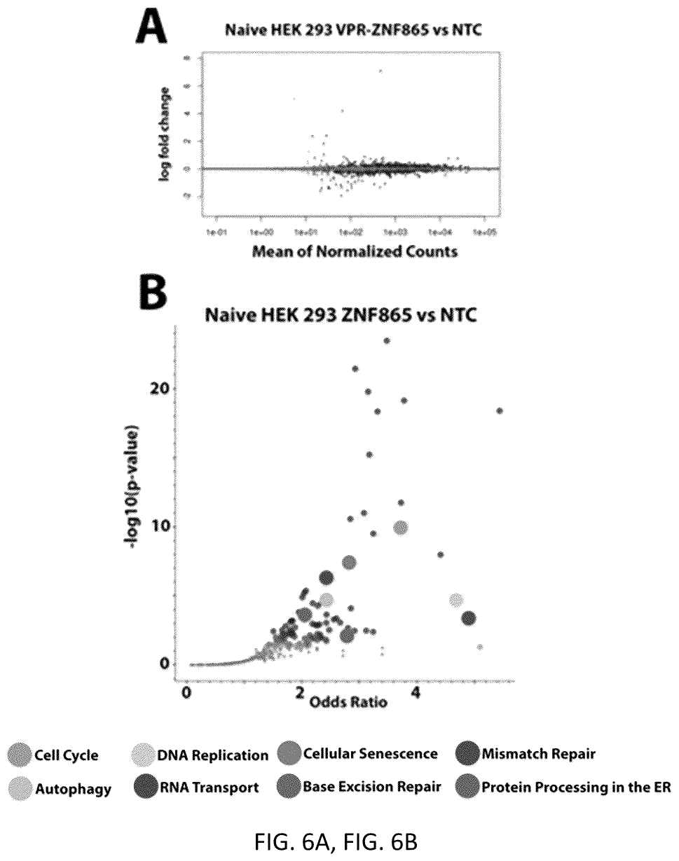

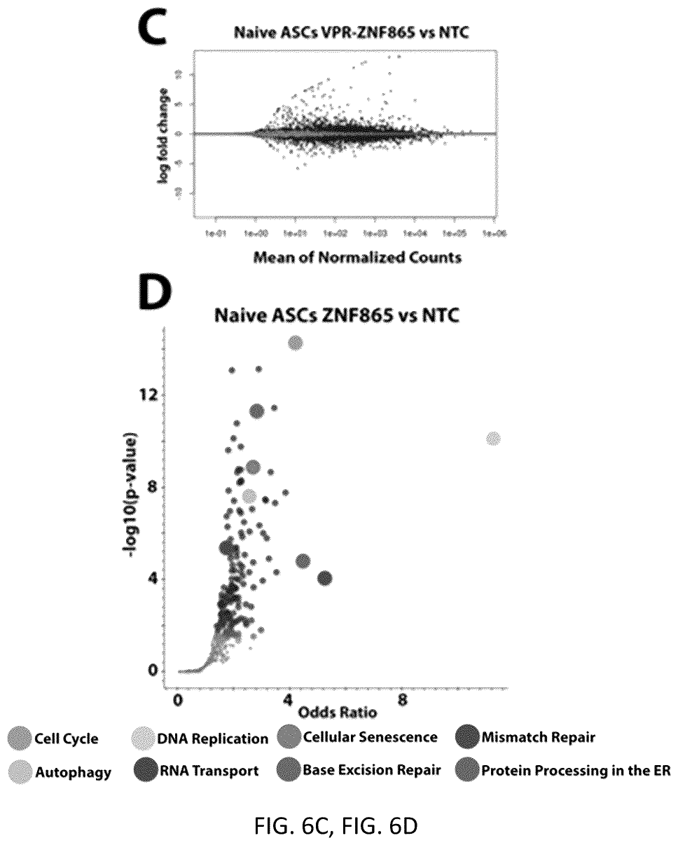

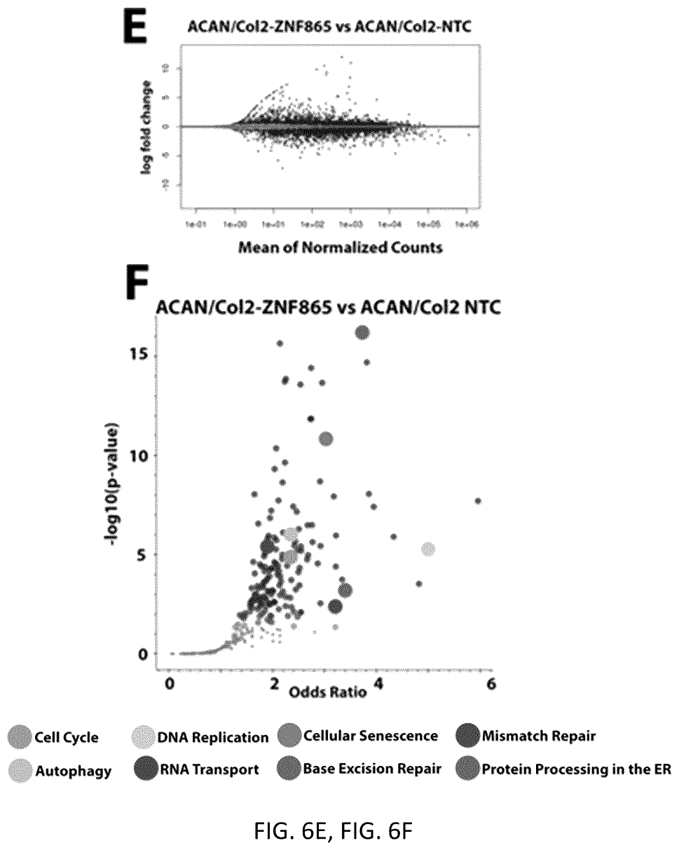

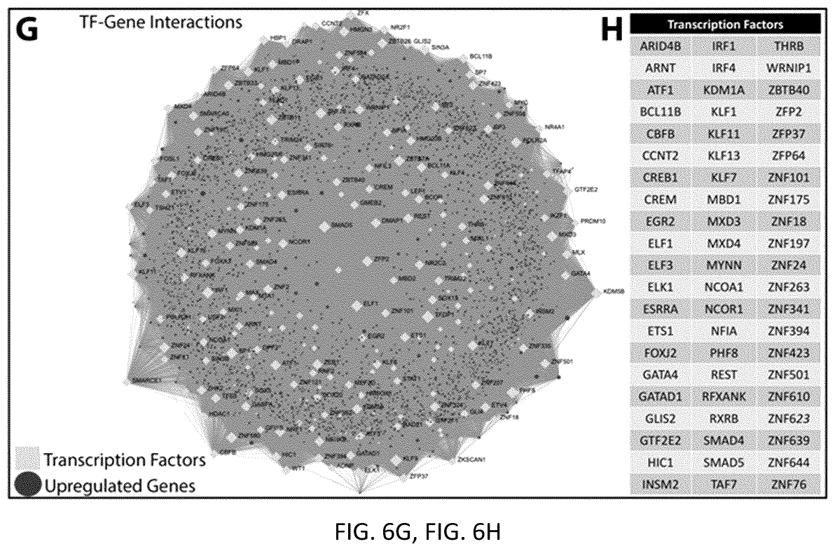

In some aspects, the target gene can be a gene involved in cell cycle, DNA replication, cellular senescence, mismatch repair, autophagy, RNA transport, base excision repair, or protein processing. In some aspects, the target gene can be a gene can be one or more of the genes shown in FIG. 6.

In some aspects, the method occurs in vivo. Thus, in some aspects, contacting a cell comprises administering to a subject comprising the cell a therapeutically effective amount of a vector comprising a nucleic acid sequence encoding a ZNF865 protein, fragment of ZNF865, or variant of ZNF865; a therapeutically effective amount of a ZNF865 protein; or one or more vectors comprising one or more nucleic acid sequences encoding a target gene-specific guide RNA (gRNA) and a dCas9 fused to ZNF865, a fragment of ZNF865, or a variant of ZNF865.

In some aspects, the gRNA hybridizes with a DNA locus (or target sequence) of a target gene in a cell. In some aspects, the target sequence is a nucleic acid sequence upstream of a transcriptional start site of the target gene, such, aggrecan or collagen II.

In some aspects, a promoter is operably linked to the nucleotide sequence encoding a target gene-specific gRNA and/or the nucleic acid sequence encoding a ZNF865 protein a fragment of ZNF865, or a variant of ZNF865. In some aspects, a regulatory element, such as, but not limited to, a promoter, is operably linked to the nucleotide sequence encoding a dCas9 fused to ZNF86, a fragment of ZNF865, or a variant of ZNF865. In some aspects, the regulatory element can be a promoter, promoter enhancer, internal ribosomal entry site (IRES) or other element that are capable of controlling expression (e.g., transcription termination signals, including but not limited to polyadenylation signals and poly-U sequences).

In some aspects, the cell is a eukaryotic cell. In some aspects, the eukaryotic cell is a mammalian cell. In some aspects, the mammalian cell is a human cell. For example, the cell can be a stem cell, bone cell, blood cell, muscle cell, fat cell, skin cell, nerve cell, endothelial cell. In some aspects, the cell is a T cell (e.g., CD4+ T cell, CD8+ T cell).

In some aspects, the one or more vectors are lentiviral, adenoviral or adeno-associated virus particles.

### K. Method of Increasing Deposition of a Target Gene

In some aspects, ZNF865 increases deposition of a target gene, such as aggrecan and/or collagen II, in the extracellular matrix of a cell.

Disclosed are methods of increasing deposition of a target gene, such as aggrecan and/or collagen II, in the extracellular matrix of a cell using gene therapy, protein therapy, or CRISPR therapy.

Disclosed are methods of increasing deposition of aggrecan and/or collagen II in the extracellular matrix of a cell comprising administering to a cell a therapeutically effective amount of a vector comprising a nucleic acid sequence encoding a ZNF865 protein, a fragment of ZNF865, or a variant of ZNF865; a therapeutically effective amount of a ZNF865 protein; one or more vectors comprising one or more nucleic acid sequences encoding a ZNF865 specific guide RNA (gRNA) and a dCas9 fused to a transcriptional activator, wherein the transcriptional activator upregulates ZNF865; or one or more vectors comprising one or more nucleic acid sequences encoding a target gene-specific guide RNA (gRNA) and a dCas9 fused to ZNF865, wherein the target gene is aggrecan and/or collagen II, wherein ZNF865, a fragment of ZNF865, or a variant of ZNF865 increases deposition of aggrecan and/or collagen II in the extracellular matrix of the cell.

In some aspects, the target gene-specific gRNA sequence can be a sequence that hybridizes to a DNA locus (e.g. target sequence) of a target gene, such as, but not limited to, aggrecan or collagen II. Examples of these gRNA sequences can be found in U.S. patent application Ser. No. 17/284,908 which is hereby incorporated by reference for its teaching of gRNA sequences that hybridizes to a target sequence of Aggrecan or Collagen II.

In some aspects, the ZNF865 specific gRNA can be any of those listed in Table 1.

In some aspects, the DNA locus, or target sequence, is a nucleic acid sequence upstream of a transcriptional start site of ZNF865, a fragment of ZNF865, or a variant of ZNF865 or of the target gene (e.g. aggrecan, collagen II).

In some aspects, the transcriptional activator can be, but is not limited to, VPR, VP64, SAM, CNP, SPH, SunTag, or p300.

In some aspects, a promoter is operably linked to the nucleotide sequence encoding a target gene-specific gRNA, ZNF865-specific gRNA, and/or the nucleic acid sequence encoding a ZNF865 protein, a fragment of ZNF865, or a variant of ZNF865. In some aspects, a regulatory element, such as, but not limited to, a promoter, is operably linked to the nucleotide sequence encoding a dCas9 fused to ZNF86, a fragment of ZNF865, or a variant of ZNF865. In some aspects, the regulatory element can be a promoter, promoter enhancer, internal ribosomal entry site (IRES) or other element that are capable of controlling expression (e.g., transcription termination signals, including but not limited to polyadenylation signals and poly-U sequences).

In some aspects, the cell is a eukaryotic cell. In some aspects, the eukaryotic cell is a mammalian cell. In some aspects, the mammalian cell is a human cell. For example, the cell can be a stem cell, bone cell, blood cell, muscle cell, fat cell, skin cell, nerve cell, endothelial cell. In some aspects, the cell is a T cell (e.g., CD4+ T cell, CD8+ T cell).

In some aspects, the one or more vectors are lentiviral, adenoviral or adeno-associated virus particles.

### L. Methods of Downregulating a Target Using ZNF865

In some aspects, ZNF865 can be used to decrease transcription of a target gene.

Disclosed are methods of downregulating expression of a target gene by using ZNF865 as a transcriptional activator in a cell using gene therapy, protein therapy, or CRISPR therapy. In some aspects, the methods can be performed in vitro or in vivo.

Disclosed are methods of downregulating expression of a target gene comprising contacting a cell with a therapeutically effective amount of a vector comprising a nucleic acid sequence encoding a ZNF865 protein, a fragment of ZNF865, or a variant of ZNF865; a therapeutically effective amount of a ZNF865 protein; or one or more vectors comprising one or more nucleic acid sequences encoding a target gene-specific guide RNA (gRNA) and a dCas9 fused to ZNF865, wherein ZNF865 protein, a fragment of ZNF865, or a variant of ZNF865 downregulates the target gene.

In some aspects, the method of downregulating expression of a target gene comprising contacting a cell with a therapeutically effective amount of a vector comprising a nucleic acid sequence encoding a ZNF865 protein, a fragment of ZNF865, or a variant of ZNF865 protein comprises using one or more of the vectors comprising a ZNF865 nucleic acid sequence described herein.

In some aspects, the method of downregulating expression of a target gene in a cell comprising contacting a cell with a therapeutically effective amount of a ZNF865 protein comprises using any of the ZNF865 proteins, variants or fragments thereof described herein. For example, any of SEQ ID NOs: 1-3 or variants thereof can be used.

In some aspects, the method of downregulating expression of a target gene in a cell comprising contacting a cell with one or more vectors comprising one or more nucleic acid sequences encoding a target gene-specific gRNA and a dCas9 fused to ZNF865, a fragment of ZNF865, or a variant of ZNF865. In some aspects, the ZNF865 upregulates the target gene. In some aspects, only the target gene-specific gRNA is delivered using a vector while the dCas9 fused to ZNF865, a fragment of ZNF865, or a variant of ZNF865 can be delivered as a protein.

In some aspects, the target gene can be a gene involved in cell cycle, DNA replication, cellular senescence, mismatch repair, autophagy, RNA transport, base excision repair, or protein processing. In some aspects, the target gene can be a gene can be one or more of the genes shown in FIG. 6. Thus, in some aspects the target sequence that hybridizes with the gRNA is a nucleic acid sequence upstream of a transcriptional start site of the target gene.

In some aspects, the target gene-specific gRNA sequence can be a sequence that hybridizes to a target sequence of a target gene, such as, but not limited to, a gene involved in cell cycle, DNA replication, cellular senescence, mismatch repair, autophagy, RNA transport, base excision repair, or protein processing. In some aspects, the target gene-specific gRNA sequence can be a sequence that hybridizes to a target sequence of a target gene, such as, but not limited to, Aggrecan or Collagen II. Examples of these gRNA sequences can be found in U.S. patent application Ser. No. 17/284,908 which is hereby incorporated by reference for its teaching of gRNA sequences that hybridizes to a target sequence of Aggrecan or Collagen II.

In some aspects, the upregulation of the target gene can be a direct regulation from the ZNF865 protein, fragment of ZNF865, or variant of ZNF865. In some aspects, the downregulation of the target gene can be an indirect regulation through other gene changes initiated by the ZNF865 protein, fragment of ZNF865, or variant of ZNF865.

In some aspects, the target gene-specific gRNA sequence can be a sequence that hybridizes to a target sequence of a target gene, such as, but not limited to, a gene involved in cell cycle, DNA replication, cellular senescence, mismatch repair, autophagy, RNA transport, base excision repair, or protein processing. In some aspects, the target gene-specific gRNA sequence can be a sequence that hybridizes to a target sequence of a target gene, such as, but not limited to, Aggrecan or Collagen II.

In some aspects, the method occurs in vivo. Thus, in some aspects, contacting a cell comprises administering to a subject comprising the cell a therapeutically effective amount of a vector comprising a nucleic acid sequence encoding a ZNF865 protein, fragment of ZNF865, or variant of ZNF865; a therapeutically effective amount of a ZNF865 protein; or one or more vectors comprising one or more nucleic acid sequences encoding a target gene-specific guide RNA (gRNA) and a dCas9 fused to ZNF865, a fragment of ZNF865, or a variant of ZNF865.

In some aspects, the gRNA hybridizes with a DNA locus (or target sequence) of a target gene in a cell. In some aspects, the target sequence is a nucleic acid sequence upstream of a transcriptional start site of the target gene, such as Aggrecan or Collagen II.

In some aspects, a promoter is operably linked to the nucleotide sequence encoding a target gene-specific gRNA and/or the nucleic acid sequence encoding a ZNF865 protein a fragment of ZNF865, or a variant of ZNF865. In some aspects, a regulatory element, such as, but not limited to, a promoter, is operably linked to the nucleotide sequence encoding a dCas9 fused to ZNF86, a fragment of ZNF865, or a variant of ZNF865. In some aspects, the regulatory element can be a promoter, promoter enhancer, internal ribosomal entry site (IRES) or other element that are capable of controlling expression (e.g., transcription termination signals, including but not limited to polyadenylation signals and poly-U sequences).

In some aspects, the cell is a eukaryotic cell. In some aspects, the eukaryotic cell is a mammalian cell. In some aspects, the mammalian cell is a human cell. For example, the cell can be a stem cell, bone cell, blood cell, muscle cell, fat cell, skin cell, nerve cell, endothelial cell. In some aspects, the cell is a T cell (e.g., CD4+ T cell, CD8+ T cell).

In some aspects, the one or more vectors are lentiviral, adenoviral or adeno-associated virus particles.

### M. Methods of Treating

Disclosed are methods of treating a subject in need thereof comprising administering one or more of the disclosed nucleic acids, vectors, proteins, or compositions. In some aspects, a subject in need thereof is a subject having degenerative disc disease. In some aspects, the subject in need thereof can be a subject having any disease, wherein the target gene being upregulated is known to help treat that disease. In some aspects, aggrecan and/or collagen II can help treat degenerative disc disease and therefore methods of treating degenerative disc disease comprising administering any of the disclosed nucleic acids, vectors, proteins, or compositions that lead to an increase in aggrecan and/or collagen II.

Disclosed are methods of treating degenerative disc disease comprising administering to a subject a therapeutically effective amount of a vector comprising a nucleic acid sequence encoding a ZNF865 protein; a therapeutically effective amount of a ZNF865 protein; a ZNF865 specific gRNA and a dCas9 fused to a transcriptional activator, wherein the transcriptional activator upregulates ZNF865; or one or more vectors comprising one or more nucleic acid sequences encoding a target gene-specific guide RNA (gRNA) and a dCas9 fused to ZNF865, wherein the target gene is aggrecan and/or collagen II.

In some aspects, the target gene-specific gRNA sequence can be a sequence that hybridizes to a DNA locus (e.g. target sequence) of a target gene, such as, but not limited to, aggrecan or collagen II. Examples of these gRNA sequences can be found in U.S. patent application Ser. No. 17/284,908 which is hereby incorporated by reference for its teaching of gRNA sequences that hybridizes to a target sequence of Aggrecan or Collagen II.

In some aspects, the ZNF865 specific gRNA can be any of those listed in Table 1.

In some aspects, the DNA locus, or target sequence, is a nucleic acid sequence upstream of a transcriptional start site of ZNF865, a fragment of ZNF865, or a variant of ZNF865 or of the target gene (e.g. aggrecan, collagen II).

In some aspects, the transcriptional activator can be, but is not limited to, VPR, VP64, SAM, CNP, SPH, SunTag, or p300.

In some aspects, a promoter is operably linked to the nucleotide sequence encoding a target gene-specific gRNA, ZNF865-specific gRNA, and/or the nucleic acid sequence encoding a ZNF865 protein, a fragment of ZNF865, or a variant of ZNF865. In some aspects, a regulatory element, such as, but not limited to, a promoter, is operably linked to the nucleotide sequence encoding a dCas9 fused to ZNF865, a fragment of ZNF865, or a variant of ZNF865. In some aspects, the regulatory element can be a promoter, promoter enhancer, internal ribosomal entry site (IRES) or other element that are capable of controlling expression (e.g., transcription termination signals, including but not limited to polyadenylation signals and poly-U sequences).

In some aspects, the cell is a eukaryotic cell. In some aspects, the eukaryotic cell is a mammalian cell. In some aspects, the mammalian cell is a human cell. For example, the cell can be a stem cell, bone cell, blood cell, muscle cell, fat cell, skin cell, nerve cell, endothelial cell. In some aspects, the cell is a T cell (e.g., CD4+ T cell, CD8+ T cell).

In some aspects, the one or more vectors are lentiviral, adenoviral or adeno-associated virus particles.

### N. Dosages

Disclosed are dosing regimens comprising administering a single dose of one or more of the disclosed compositions, vectors, nucleic acid sequences or polypeptides to a subject in need thereof, wherein the single dose comprises an amount effective to increase expression of ZNF865 or a target gene.

Disclosed are dosing regimens comprising administering at least one, two, three, or four doses of one or more of the disclosed compositions to a subject in need thereof, wherein each dose is the same concentration. In some aspects, each dose after a first dose can be decreased. In some aspects, each dose after a first dose can be increased.

In some aspects, a single dose can be a continuous administration. In some aspects, a continuous administration can be hours, days, weeks, or months. In some aspects, there can be two or more doses. In some aspects, the two or more doses can be administered days, weeks, or months apart.

### O. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more of the disclosed compositions, proteins, nucleic acid sequences or vectors.

### Examples

### A. Example 1

### 1. Introduction:

Degenerative disc disease (DDD) is the leading cause of disability worldwide, characterized by the breakdown of intervertebral discs (IVD). Stem cell therapies using adipose-derived stem cells (ASCs) are promising options to treat DDD because of stem cells' ability to regenerate and restore functional tissue to the IVD. Recently, CRISPR-guided activation (CRISPRa) genome-wide perturbation screens have been used to profile gene function. Using a CRISPRa screen, a novel gene, MaB1, was identified that increases target gene expression. By multiplex upregulating MaB1 in our CRISPRa ACAN/Col2 cell line, extracellular matrix deposition of aggrecan and collagen-II may be increased.

### 2. Materials and Methods:

ACAN/Col2-upregulated ASCs were transduced with a lentivirus containing sgRNA targeting a nontarget control (NTC) or MaB1. ACAN/Col2-NTC and MaB1 upregulated cells were pellet cultured to examine ECM deposition. Pellets were cultured for 21-days with media changes every 2-3 days. After 21-days pellets were harvested, fixed, and submitted for histology (n=5), or papain digested and designated for biochemical analysis (n=10). Histological sections were stained for sulfated glycosaminoglycan deposition (sGAG). Papain digested pellets were analyzed for hydroxyproline, sGAG, and DNA content. Statistical significance was determined using a student's t-test.

i. RNA Sequencing

To evaluate differential gene expression due to ZNF865 upregulation, RNA sequencing will be performed. ZNF865 will be upregulated in singleplex and multiplex to evaluate gene expression changes. Briefly, HEK293 cells and naïve ASCs that contain the VPR-Puro upregulation expression cassette will be transduced with the guide delivery plasmid targeting the upregulation system to ZNF865[1]. Furthermore, an ACAN/Col2 upregulated cell line will be transduced with ZNF865 to evaluate gene expression changes in multiplex. Using previously described methods, data can be normalized and compared to a naïve cell line that does not contain ZNF865 edits or ACAN/Col2 upregulation alone[1,2]. Briefly, RNA was isolated from samples using a Quick-RNA Kit (Zymo Research, Irvine, CA), isolated RNA was prepared using an Illumina TruSeq Stranded mRNA Kit, and samples were submitted to the High-Throughput Genomics Shared Resource Core at the Huntsman Cancer Institute for RNAseq. Sequencing reads were aligned to hg38 build of the human genome, and reads mapping UCSC known genes were counted using feature counts from the SubRead package[3,4]. Reads were normalized and differential analysis was performed using DESeq2[3,5]. Enriched GO biological and molecular processes were determined from genes significantly upregulated using Enrichr[6-8].

ii. Pellet Culture

To evaluate ECM deposition in ACAN/Col2 edited cells, 3D pellet culture analysis was performed. Briefly, pellets were formed by resuspending ACAN/Col2-NTC and ACAN/Col2-ZNF865 edited ASCs at a concentration of 1.25 million cells/mL in a serum-free growth medium. 200 μL of the cell suspension was pipetted into individual wells of a 96-well v-bottom plate and spun at 270 G for 5 minutes at 4° C. Cells were allowed to contract overnight and form pellets. The following day pellets were gently lifted from the bottom of the plate. Media was changed on the pellets every 2-3 days for 21-days. After 21-days pellets were harvested and either papain digested for biochemical analysis or fixed in formalin and submitted for histological analysis, as previously described [1,9].

iii. Hydroxyproline Assay

The total amount of collagen content contained within papain digested pellets and supernatant (n=10) was analyzed using a modified hydroxyproline assay [1]. Hydroxyproline assay was performed as previously described, with the same adjustments for hydrolysis time and adjusting the pH of the oxidation buffer to 6.5 with glacial acetic acid [1].

iv. Sulfated Glycosaminoglycan Quantification

The total amount of sGAG content within papain digested pellets and supernatant (n=10) was analyzed using dimethylmethylene blue assay [1,10].

v. Macroscopic Pellet Imaging

After 21-days of pellet culture, pellets were imaged in their respective wells for qualitative comparison of gross pellet morphology.

vi. Histological Analysis

To prepare pellets for staining, pellets (n=5) were fixed in a 10% neutral-buffered formalin solution for 24-hours. Pellets were then embedded in paraffin, sliced into 4 μm sections, and mounted on glass slides. Mounted slides were stained using alcian blue to evaluate proteoglycan deposition and counterstained with nuclear fast red to identify cell nuclei, as previously described [1,9].

vii. Cell Proliferation and Cell Death Analysis

HEK293T and ASCs expressing VPR-Puro upregulation expression cassette (n=4) were plated in a 24-well plate and transduced with ZNF865 or NTC upregulation expression cassette as previously described in Virus Transduction. 48-hours after transduction images were obtained in the center of each well to evaluate cell proliferation. Images were obtained every subsequent day for 5 days.

Naïve HEK293 and Naïve ASCs (n=4) were plated in a 24-well plate and transduced with KRAB-ZNF865 knockdown or KRAB-NTC expressing lentivirus as previously described in Virus Transduction. 48-hours after transduction images were obtained in the center of each well to evaluate cell death. Images were obtained every subsequent day until cell death was observed.

viii. Cell Cycle Analysis

ZNF865 and NTC edited cells were grown to confluence in a T-75 flask, lifted from the flask, and resuspended in growth medium at a concentration of 1 million cells/mL and 2 drops of Vybrant™ DyeCycle™ Violet Ready Flow™ Reagent (ThermoFisher Scientific, R37172) was added to the suspension before being incubated at 37° C. for 30 minutes, following manufacturer's instructions. Following incubation, cells were analyzed on a Cytoflex S Flow Cytometer (Beckman Coulter Life Sciences, Indianapolis, IN). Flow cytometry data was analyzed using FlowJo Flow Cytometry Software (BD Biosciences).

### 3. Results and Discussion:

MaB1 upregulated cells show a significant increase in volume compared to the NTC. Alcian blue staining shows an increase in sGAG deposition in MaB1 edited pellets compared to the NTC and biochemical analysis shows a significant increase in Total sGAG and percent of sGAG retained within the pellet for MaB1 compared to the NTC. Biochemical analysis for hydroxyproline content shows a significant increase in Total Collagen and Percent collagen retained in MaB1 edited pellets compared to the NTC.

i. Pellet Culture

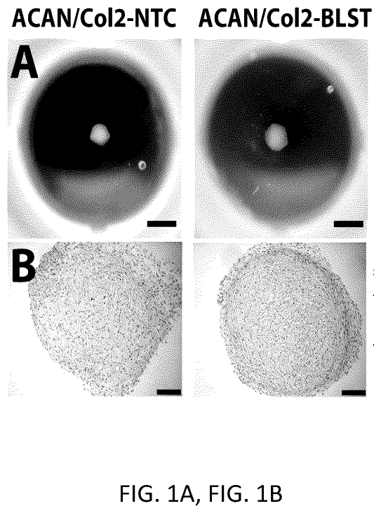

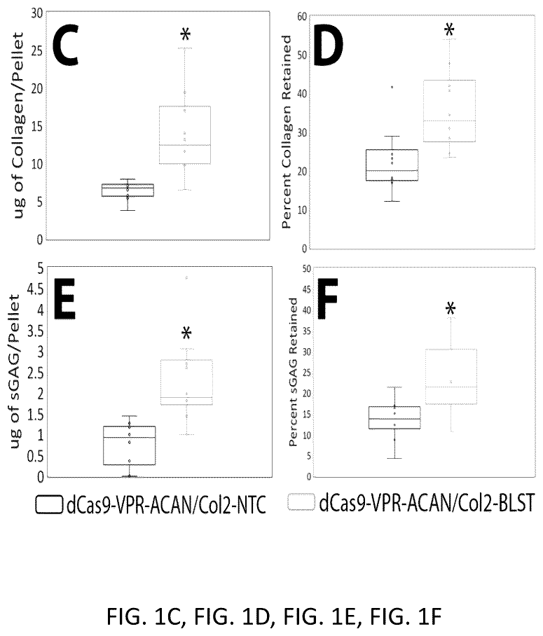

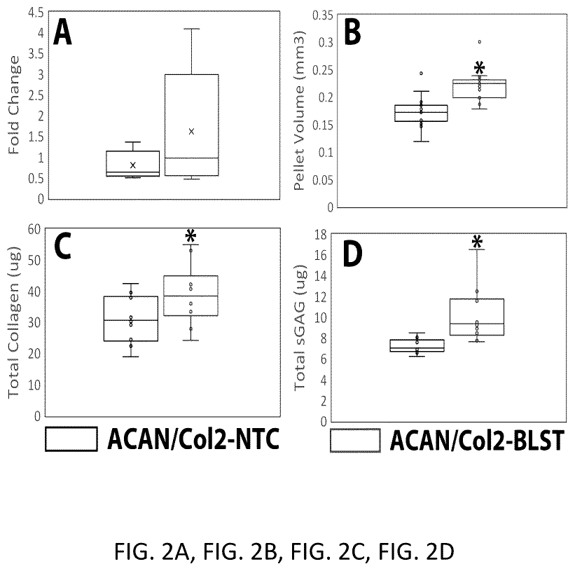

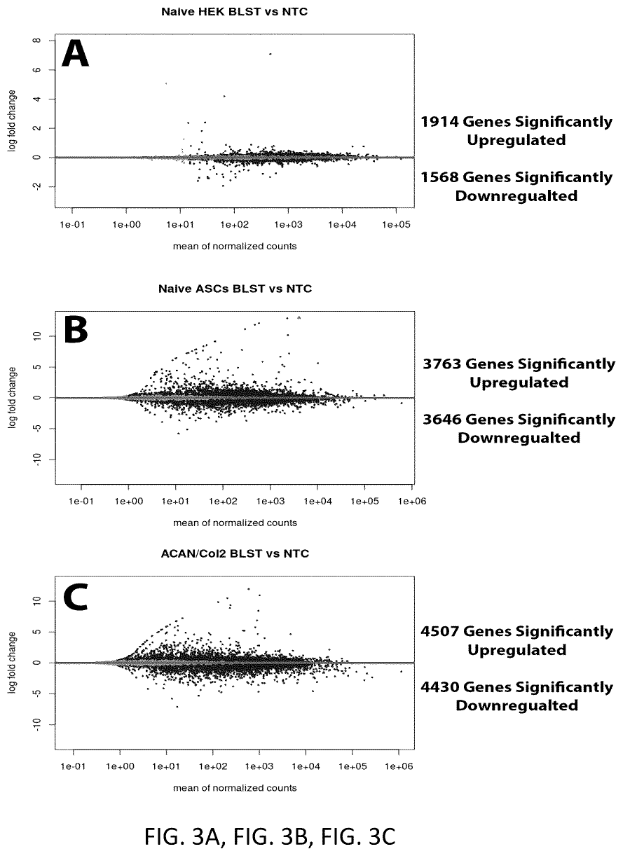

Pellet culture analysis showed increased extracellular matrix (ECM) deposition in ZNF865-edited cells compared to the nontarget control. There are noticeable increases in pellet volume (FIG. 1A) and sGAG deposition in (FIG. 1B) compared to the nontarget control. Additionally, there are significant increases in micrograms of collagen per pellet (FIG. 1C), percent of collagen retained within the pellets (FIG. 1D), micrograms of sGAG per pellet (FIG. 1E), and percent of sGAG retained per pellet (FIG. 1F). Overall, these results display increased tissue deposition and retention of key ECM molecules within the pellet. Additionally, qRT-PCR data verifies upregulation of ZNF865 in ACAN/Col2-ZNF865 edited cells compared to ACAN/Col2-NTC edited cells (FIG. 2A), and analysis showed significant increases in pellet volume in ZNF865-edited cells compared to the nontarget control, as well as increased total collagen and total sGAG concentrations contained in the pellet and within the supernatant. Overall, these results show that upregulation of ZNF865 significantly changes cell phenotype and improves cartilaginous ECM tissue deposition.

ii. RNA Sequencing Analysis

Differentially expressed genes were analyzed using RNA sequencing to evaluate changes in cell phenotype due to increased ZNF865 expression. Naïve HEK293T cells show 1,914 genes that are significantly upregulated and 1,568 genes that are significantly downregulated due to ZNF865 upregulation (FIG. 2A). Naïve ASCs show 3,763 genes that are significantly upregulated and 3,646 genes that are significantly downregulated due to ZNF865 upregulation (FIG. 2B). VPR-ACAN/Col2-ZNF865 ASCs show 4,507 genes that are significantly upregulated and 4,430 genes that are significantly downregulated due to ZNF865 upregulation (FIG. 2C). Overall, increasing ZNF865 expression has a significant effect on cell phenotype with thousands of genes being differentially expressed in three different cell types.

iii. Cell Cycle Analysis

Upregulation of ZNF865 in naïve HEK293T cells and naïve ASCs shifted the cell cycle towards the S/G2/M phases (FIG. 1). Histograms of flow cytometry cell cycle analysis showed a shift in the cycle for ZNF865-edited cells in HEK293T cells (FIG. 1A/B) and ASCs (FIG. 1B/C) compared to a nontarget control. Quantified flow cytometry data shows percentages of cells in the G0/G1 phase of their cell cycle of the S/G2/M phase, with a robust shift towards S/G2/M phase for both ZNF865-edited cell lines (FIG. 1E).

iv. Cell Proliferation

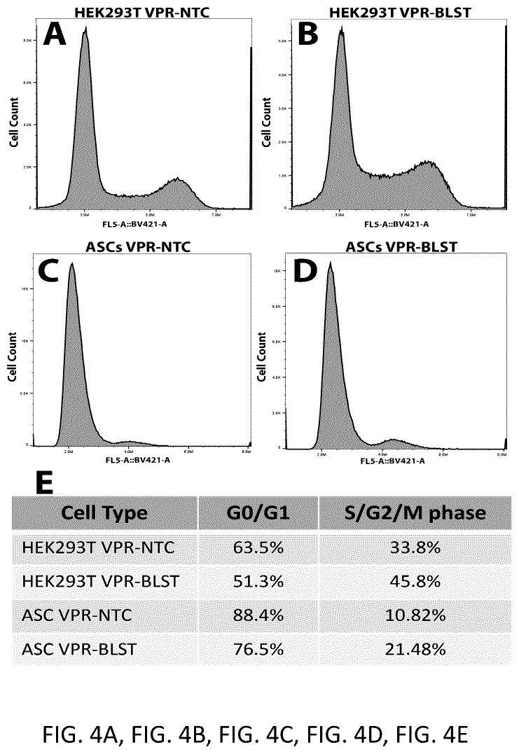

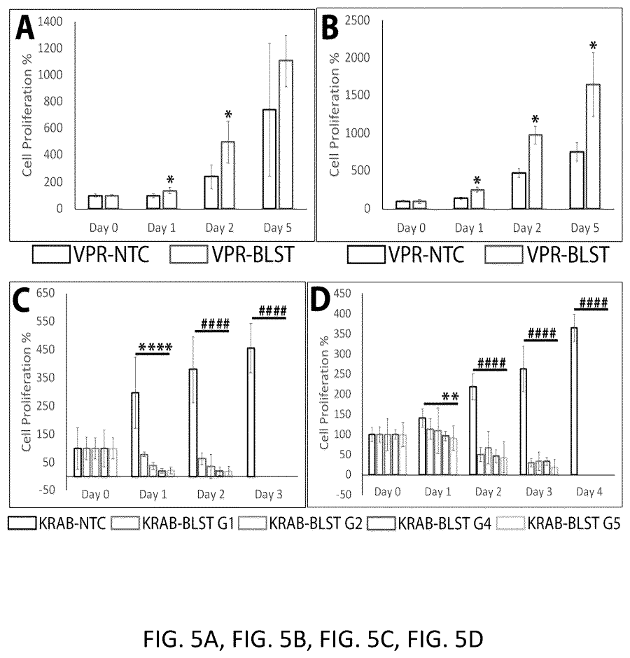

Upregulation of ZNF865 increases cell proliferation rates in naïve HEK293T cells and ASCs. Upregulation of ZNF865 in naïve HEK293T cells (FIG. 4A) and naïve ASCs (FIG. 4B) showed increased cell proliferation compared to the nontarget control group. This data is consistent with the cell cycle analysis data where there is a robust shift in the number of cells within the different phases of the cell cycle, with fewer cells in G0/G1 phase and more in the S/G2/M phase of the cycle.

v. Cell Death

Suppression of ZNF865 using the KRAB effector molecule showed cell death in both naïve HEK293T cells (FIG. 4C) and naïve ASCs (FIG. 4D) within days after transduction. These results indicate that ZNF865 is a necessary gene for cell survival.

### 4. Conclusions:

This data shows the effect of multiplex upregulating ACAN/Col2 cells with a novel gene that increases targeted gene expression. Upregulating MaB1 increases baseline ECM production in the ACAN/Col2 CRISPRa cell line to above the baseline levels seen in naïve cells dosed with growth factors. This date presents increased target gene expression without exogenous growth factors which can be used in targeted cell therapies to treat DDD and tissue engineering applications.

Those skilled in the art will recognize, or be able to ascertain using no more than routine experimentation, many equivalents to the specific embodiments of the method and compositions described herein. Such equivalents are intended to be encompassed by the following claims.

### B. Example 2

### 1. Methods

i. General Cell Culture

Complete growth medium for cell culture for HEK-293 cells consists of HG-DMEM (ThermoFisher Scientific, Waltham, MA) supplemented with 10% fetal bovine serum (FBS) (ThermoFisher Scientific), 25 μg/mL gentamicin (Corning), and 25 mM HEPES (ThermoFisher). Complete growth medium for culturing of human adipose derived stem cells (hASCs) consists of Lonza ADSC Basal Medium (Lonza, Lexington, MA PT-3273), 10% MSC FBS (ThermoFisher Scientific), 5 mL GlutaMax (ThermoFisher Scientific), 30 μg/mL Gentamicin (Corning), and 15 ng/mL Amphotericin (ThermoFisher Scientific). Complete growth medium for cell culture of Jurkat cells consists of Advanced RPMI 1640 (ThermoFisher Scientific) supplemented with 10% FBS (ThermoFisher Scientific), 10 mM HEPES (ThermoFisher Scientific), and 100 U/mL penicillin and 100 μg/mL streptomycin (ThermoFisher Scientific). Primary human nucleus pulposus cells (hNPCs) were cultured in complete growth medium containing DMEM-HG (ThermoFisher Scientific) supplemented with 10% FBS (ThermoFisher Scientific), 50 μg/mL gentamicin (Corning), 25 mM HEPES (ThermoFisher Scientific), and 2 ng/mL of recombinant human fibroblast growth factor-basic (rhFGF, Peprotech, Cranbury, NJ, 100-18° C.). All cell culture was performed in standard culture conditions (21% O₂, 5% CO₂, 37° C.), with media changes every 2-3 days.

ii. Harvesting of hNPCs and Culture

Nucleus pulposus (NP) tissue was obtained from surgical tissue waste and hNPC isolation was isolated as previously described [1]. Briefly, NP tissue was rinsed twice with washing medium (DMEM-HG, 165 μg/mL gentamicin sulfate, 100 μg/mL kanamycin sulfate, 1.25 μg/mL amphotericin), minced, and enzymatically digested in washing medium with 0.3% (w/v) collagenase type II (Worthington Biochemical), 0.2% pronase (Sigma), and 5% FBS (ThermoFisher Scientific) for 2-3 hours at 37° C. with 5% CO₂ under gentle agitation[1].

Isolated cells were passed through a 70 m cell strainer and washed twice. Cells were counted and plated at a density of 10,000 cells/cm² in NP cell culture medium (DMEM-HG with 10% FBS, 50 μg/mL gentamicin, 25 mM HEPES) supplemented with fresh 2 ng/mL fibroblast growth factor-2 (FGF-2) (Peprotech). Cells were cultured in this medium at 37° C. and 5% CO₂ in a humidified atmosphere and subcultured to 90% confluency, as previously described [1].

iii. gRNA Design

Upregulation gRNA was determined using a previously analyzed CRISPRa genome wide screen by selecting the top-ranked gRNA from the pool of five. The top performing gRNA was used for the duration of the upregulation studies.

Downregulation gRNA design was performed using Genome Target Scan 2 (GT-Scan2) and ChopChop [2,3]. The 5′-UTR and the promoter region up to 1000 bp upstream of the ZNF865 transcription start site (TSS) were analyzed for gRNAs. The top 5 gRNAs from each analysis tool, with the least number of off-target sites, were compared and inspected using the BLAT tool of the UCSC genome browser to ensure minimal overlap between gRNAs [4]. A total of 5 gRNAS were selected for validation in vitro, as well as a gRNA that does not target the human genome (nontarget control/NTC). Oligos for ZNF865 gRNAs were synthesized. These sgRNAs, and a nontarget control (NTC) that does not target the human genome, were synthesized, annealed, phosphorylated, and ligated into individual sgRNA lentiviral upregulation expression vectors (Addgene, #83919) or KRAB CRISPRi downregulation expression vector pLV-hUbC-dCas9 KRAB-T2A-GFP (Addgene, #67620). Successful gRNA insertion was verified through Sanger sequencing. The gRNA plasmid DNA was used to produce lentivirus.

TABLE 2
sgRNA primers for targeting the CRISPR system to ZNF865 and a non-
target control for upregulation and downregulation.

| gRNA | Primer Sequence(s) |
|---|---|
| CRISPR-activation gRNA | Forward Primer 5′-ccgcacaaggatggatgagt-3′ (SEQ ID |
| | NO: 422) |
| | Reverse Primer 3′-actcatccatccttgtgcgg-5′ (SEQ ID NO: 423) |
| KRAB-ZNF865 Guide 1 | Forward Primer 5′-atgggggcgcacgactgcta-3′ (SEQ ID |
| | NO: 424) |
| | Reverse Primer 3′-tagcagtcgtgcgcccccat-5′ (SEQ ID NO: 425) |
| KRAB-ZNF865 Guide 2 | Forward Primer 5′-gacgcccagagcgtgtcgcg-3′ (SEQ ID |
| | NO: 426) |
| | Reverse Primer 3′-cgcgacacgctctgggcgtc-5′ (SEQ ID |
| | NO: 427) |
| KRAB-ZNF865 Guide 3 | Forward Primer 5′-tcgcccaccggaatcggccc-3′ (SEQ ID |
| | NO: 428) |
| | Reverse Primer 3′-gggccgattccggtgggcga-5′ (SEQ ID |
| | NO: 429) |
| KRAB-ZNF865 Guide 4 | Forward Primer 5′-ccatgcggaatagaagccct-3′ (SEQ ID |
| | NO: 430) |
| | Reverse Primer 3′-agggcttctattccgcatgg-5′ (SEQ ID NO: 431) |
| KRAB-ZNF865 Guide 5 | Forward Primer 5′-gattatagcgattatatgag-3′ (SEQ ID NO: 432) |
| | Reverse Primer 3′-ctcatataatcgctataatc-5′ (SEQ ID NO: 433) |
| Non-target | Forward Primer 5′-tttttaatacaaggtaatct-3′ (SEQ ID NO: 434) |
| | Reverse Primer 3′-agattaccttgtattaaaaa-5′ (SEQ ID NO: 435) |

iv. Lentivirus Production

The gRNA plasmid DNA was used to produce lentivirus, as previously described[1,5,6]. The amplified gRNA plasmid DNA was co-transfected into HEK 293T cells with psPAX2 (Addgene, plasmid #12260) and pMD2.G (Addgene, plasmid #12259) lentiviral packaging plasmids to create a lentivirus, as previously described[1]. Briefly, HEK 293T cells were seeded at a density of 62,500 cells/cm². The following day, the lentiviral plasmids were added to the cells with Lipofectamine 2000 (ThermoFisher Scientific), following the manufacturer's protocol. After 24 hours, the cell supernatant was discarded and replaced with fresh medium. Cell supernatant containing the lentiviral library vectors was collected at 48 and 72 hours, concentrated to 10× or 100×, and stored at −80° C. until use [16].

### Lentivirus Transduction

v. CRISPRa Upregulation

dCas9-VPR expressing HEK-293 cells, ASCs, and dCas9-VPR-ACAN/Col2 ASCs were plated at a density of 20,000 cells/cm² for HEK 293 cells and 5,000 cells/cm² for ASCs in a 24-well plate and allowed to attach overnight. Jurkat cells expressing dCas9-VPR were plated in a 24-well plate at a density of 50,000 cells/mL. The following day, gRNA virus was diluted 1:25 in complete growth medium supplemented with 4 μg/mL polybrene and used to transduce cells. All transduced cells were examined for fluorescence after 48 hours and showed near 100% transduction efficiency. Jurkat cells expressing dCas9-VPR were plated in a 24-well plate at a density of 50,000 cells/mL.

vi. CRISPRi Downregulation

For ZNF865 downregulation, naïve HEK-293, naïve ASCs, and hNPCs (passage 2) were plated at a density of 20,000 cells/cm², 5,000 cells/cm², or 10,000 cells/cm² respectively, in 24-well plates and allowed to attach overnight [1]. The following day, 100× sgRNA virus was diluted 1:16 in complete growth medium supplemented with 4 μg/mL polybrene and used to transduce cells. Cells were examined for fluorescence after 72-hours and showed near 100% transduction efficiency.

vii. RNA Sequencing

RNA-sequencing (RNA-seq) was utilized to evaluate differential gene expression due to ZNF865 upregulation. ZNF865 was upregulated in singleplex and multiplex to evaluate differential gene expression changes, as previously described [5]. HEK-293 cells and naïve ASCs that contain the VPR-Puro upregulation expression cassette were transduced with the guide delivery plasmid targeting the upregulation system to ZNF865 or a NTC that does not target a gene in the human genome [5]. Furthermore, our previously developed ACAN/Col2 upregulated cell line was transduced with ZNF865 to evaluate gene expression changes in multiplex. Cells were transduced, as described in Lentivirus Transduction section, and cultured until near confluence prior to RNA isolation. Briefly, Total RNA was isolated from samples using a Quick-RNA Kit (Zymo Research, Irvine, CA). Isolated Total RNA was prepared using an Illumina TruSeq Stranded mRNA Kit, and samples were submitted to the High-Throughput Genomics Shared Resource Core at the Huntsman Cancer Institute for RNA-seq.

Using previously described methods, data was normalized and compared to control cells that do not contain ZNF865 edits [1,5]. Sequencing reads were aligned to hg38 build of the human genome and reads mapping UCSC known genes were counted using featureCounts from the SubRead package [7,8]. Reads were normalized and differential analysis was performed using DESeq2 [7,9]. Enriched GO biological, molecular processes, and cell types were determined from significantly regulated genes using Enrichr [10-12].

viii. Verification of ZNF865 Upregulation and Downregulation

Transduced cells were analyzed for changes in ZNF865 gene expression by qRT-PCR (n=4). 72-hours post-transduction, RNA was isolated and harvested using the Quick-RNA Micro Kit (Zymo Research, R1051), complementary DNA (cDNA) was synthesized from the purified RNA with high-capacity cDNA reverse transcription kit with RNAse inhibitor (Applied Biosystems). cDNA was then used for qRT-PCR with TaqMan gene expression assays (ThermoFisher) for ZNF865 (Hs05052648_s1). Beta-2-microglobulin (B2M, Hs00187842_ml) was used as an internal standard and changes in ZNF865 expression was normalized to B2M expression [13]. Fold-change in mRNA expression relative to the NTC or ZNF865 edited cells was calculated using the ΔΔCT method.

ix. Cell Proliferation Quantification

Following successful transduction, HEK-293, hASCs, hNPCs, and Jurkat cells transduced with either the CRISPRa upregulation system or CRISPRi downregulation system (n=4) were evaluated for cell proliferation over the course of 3-10 days. Individual cells were counted using ImageJ or a hemacytometer [14]. Briefly, fluorescing cell counts were obtained by uploading images into a stack and thresholding the images to ensure only individual cells are shown within the image. After thresholding, cell counts were obtained by analyzing particles, generating a mask, and then counting the masks generated while excluding cells on the edges of the image. In instances where cell density was too great for thresholding and visually isolating individual cells, cells were manually counted in ImageJ using the cell counter plugin [14].

x. Cell Cycle Analysis

For upregulation cell cycle analysis, ZNF865 and NTC edited cells were grown to confluency in a T-75 flask, lifted from the flask, and resuspended in growth medium at a concentration of 1 million cells/mL. Following the manufacturers instructions, 2 drops of Vybrant™ DyeCycle™ Violet Ready Flow™ Reagent (ThermoFisher Scientific, R37172) was added to the suspension before being incubated at 37° C. for 30 minutes. Following incubation, cells were analyzed on a Cytoflex S Flow Cytometer (Beckman Coulter Life Sciences, Indianapolis, IN). Flow cytometry data was analyzed using FlowJo Flow Cytometry Software (BD Biosciences). Gates for selecting individual cells, dsRed expressing cells, and DyeCycle™ violet expressing cells were used to isolate and analyze our cells of interest.

For downregulation cell cycle analysis, HEK 293 cells were plated in a 6-well plate at a density of 50,000 cells/mL. The following day, gRNA virus was diluted 1:25 in complete growth medium supplemented with 4 μg/mL polybrene and used to transduce cells. All transduced cells were examined for fluorescence after 48 hours and showed near 100% transduction efficiency. 72-hours after transduction, cells were lifted and resuspended in growth medium at a concentration of 1 million cells/mL and cell cycle was analyzed as described above.

xi. Cellular Senescence

Primary hNPCs at passage 2 were plated in a 24-well plate at a concentration of 10,000 cells/cm² and allowed to attach overnight. The following day attached cells were transduced with the ZNF865 or NTC downregulation expression cassettes. Cells were then cultured for 10 or 24 days, at which point they were fixed and stained for SA-β-galactosidase using a Senescent Cell Histochemical Staining Kit (Sigma-Aldrich, CS0030-1KT). Cellular senescence was quantified by counting SA-β-galactosidase stained and unstained cells using ImageJ.

Primary hNPCs at passage 1 were plated into cell culture chamber slides at a concentration of 10,000 cells/cm² and then transduced with the ZNF865 or NTC downregulation expression cassettes. Cells were then cultured for 10 or 24 days after which the cells were fixed with 10% formalin. Following fixation cells were permeabilized with 0.2% Triton X in PBS for 5 min. Cells were then washed three times with PBS and treated with blocking solution (5% goat serum [MP Biomedicals] in PBS for 1 hour at room temperature). Following treatment with blocking solution, the blocking solution was removed, and the cells were treated with P16-INK4A antibody (1:100 in 1% BSA in PBS; Proteintech) or P21 antibody (1:100 in 1% BSA in PBS; Proteintech), normal rabbit IgG (Invitrogen) or BSA only negative controls and incubated at room temperature for 2 hours. Following incubation cells were washed three times with PBS, treated with goat anti-rabbit Coralite 594 (1:100 Proteintech) in 1% BSA in PBS, and incubated for 1 hour at room temperature. Subsequently, cells were rinsed three times with PBS, treated with DAPI solution (3 ng/mL) for 10 minutes, and mounted with Vectamount Mounting Medium prior to fluorescence imaging (Olympus IX73).

xii. Pellet Culture of hASCs

To evaluate extracellular matrix (ECM) deposition in our ACAN/Col2 edited cells, 3D pellet culture analysis was performed as previously described [5,6]. Briefly, pellets were formed by resuspending ACAN/Col2-NTC and ACAN/Col2-ZNF865 edited hASCs at a concentration of 1.25 million cells/mL in a serum-free growth medium and 200 L aliquots of the cell suspension was pipetted into individual wells of a 96-well u-bottom plate and spun at 270 G for 5 minutes at 4° C. Cells were allowed to contract overnight and form pellets. The following day pellets were gently lifted from the bottom of the plate. Media was changed on the pellets every 2-3 days for 21-days and supernatant was collected during every media change. After 21-days pellets were harvested and either papain digested for biochemical analysis or fixed in formalin and submitted for histological analysis, as previously described [5,6].

xiii. Pellet Biochemical Analysis

The total amount of collagen content contained within papain digested pellets and supernatant (n=10) was analyzed using a modified hydroxyproline assay [5,6]. Hydroxyproline assay was performed as previously described, with the same adjustments for hydrolysis time and adjusting the pH of the oxidation buffer to 6.5 with glacial acetic acid [5,6]. The total amount of DNA content and sGAG content within papain digested pellets and supernatant (n=10) was analyzed using a previously described Hoescht dye assay and a dimethylmethylene blue (DMMB) assay.

xiv. Macroscopic Pellet Imaging and Size Analysis

After 21-days of pellet culture, pellets were imaged (Canon Rebel T3) in their respective wells for qualitative comparison of gross pellet morphology, as previously described [5,6]. Volume was estimated by assuming the pellets were spheres, measuring the average diameter across the pellet, and calculating volume (n=15).

xv. Pellet Culture Histological Analysis

To prepare pellets for staining, pellets (n=5) were fixed in a 10% neutral-buffered formalin solution for 24-hours, embedded in paraffin, and 5 m sections were mounted on glass slides [5,6]. Sections for each sample were stained with alcian blue (pH 2.5; Newcomer Supply) and counterstained in Nuclear-fast red (Newcomer Supply). Briefly, slides were deparaffinized and rehydrated to distilled water, suspended in 3% acetic acid for 3 minutes, suspended in Alcian blue solution at room temperature for 30 minutes, washed in distilled water for 2 minutes, suspended in Nuclear-fast red solution for 5 minutes, washed in tap water, dehydrated, cleared, and cover slipped.

xvi. Jurkat T-Cell Activation

VPR-ZNF865 and -NTC expressing Jurkat cells were plated in a 24-well plate at a density of 250,000 cells/mL (n=5). The following day 0.5 mL of media with 1× Concanavalin A (00-4978-93, ThermoFisher Scientific) was added to stimulate T-cell differentiation. Following 3 days of incubation, T-cell proliferation was evaluated using CCK8, following the manufacturers protocol, and T-cell supernatant was harvested and analyzed for IL-2 (900-M12, ThermoFisher Scientific) and IFN-γ (900-M27, ThermoFisher Scientific) cytokine secretion using an Enzyme-linked immunosorbent assay (ELISA) following the manufacturer's protocol.

xvii. DAPS Cell Seeding and Culture

Naïve ASCs, ACAN/Col2-NTC, and ACAN/Col2-ZNF865 edited ASCs were evaluated for cartilage deposition in vitro in DAPS over 5-weeks of culture. DAPS were fabricated, seeded, and cultured as previously described [17]. Briefly, nucleus pulposus (NP) region of the DAPS was formed by suspending ASCs in chemically defined media at a density of 40×10∧6 cells/mL mixing with molten 4% w/v agarose (49° C., Type VII, Sigma-Aldrich), and then cast into 6-well plates to generate agarose slabs at a final density of 20×20⁶ cells/mL and 2% m/v agarose gel. Sterilized biopsy punches generated NP regions of DAPS that were 3 mm thick and 5 mm in diameter. NP regions were cultured in isolation for 2.5 weeks prior to combining with the annulus fibrosus (AF) region of the DAPS. AF regions of DAPS were fabricated using poly(s-caprolactone) (PCL) dissolved in a 1:1 mixture of tetrahydrofuran and N,N-dimethylformamide electrospun onto a grounded, rotating mandrel. ASCs were suspended in growth medium and seeded onto PCL strips at a density of 1.5×10⁶ cells per side and cultured for 1-week prior to wrapping the strips around a custom mold and cultured on an orbital shake. Following 1.5-weeks of culture around the mold, AF regions were removed from the mold and combined with NP regions, at which point combined NP and AF regions of DAPS were cultured for 2.5-weeks.

DAPS were evaluated statistically by a Pearson's Chi-Squared Analysis with DAPS either being considered Success or Fail. Successful DAPS were DAPS that produced sufficient ECM to maintain shape and not have the AF region begin to unroll. Failed DAPS were DAPS that did not maintain shape, AF began to unroll, and had to be pinned together during the 5-week culture period.

xviii. DAPS Histological Evaluation

For histological assessment of matrix deposition after 5-weeks of culture, DAPS were fixed in 10% neutral buffered formalin, embedded in paraffin, and sectioned in the sagittal plane to 7 μm thickness. Sections were stained for sGAG deposition using Alcian blue and/or collagen deposition using picrosirius red.

xix. Statistics

RNA-seq statistical analysis was performed and described in their respective section (Enrichr, DESEQ2, α=0.05). Statistical analysis of qRT-PCR and biochemical data was performed using the RealStats plugin in Microsoft Excel using a one-way analysis of variance (ANOVA) with a Tukey's post-hoc analysis (α=0.05 for all tests).

### 2. Results

ZNF865 Upregulation Differentially Expresses Thousands of Genes

RNAseq was utilized to evaluate the global changes in gene expression due to upregulation of ZNF865 in three distinctly different cell types: HEK 293 cells, ASCs, and ACAN/Col2 CRISPRa upregulated ASCs. Upregulation of ZNF865 in HEK 293 cells, ASCs, and ACAN/Col2 ASCs significantly differentially expresses 2,699, 6,647, and 8,093 genes, respectively (FIG. 6A). In HEK 293 cells, 1512 number of genes were significantly upregulated and 1,187 were downregulated. ASCs had 3,384 upregulated genes and 3,263 downregulated genes. In ACAN/Col2 ASCs 4,041 genes were upregulated and 4,052 genes were downregulated.

Based off all the significantly differentially expressed genes gene ontologies (GO) were evaluated to examine the top 8 common molecular processes affected by these thousands of significantly differentially regulated genes (FIG. 6B). Utilizing the KEGG 2021 Human Pathway, across cell types the top molecular processes affected by ZNF865 upregulation are: cell cycle, DNA replication, cellular senescence, mismatch repair, autophagy, RNA transport, base excision repair, and protein processing in the ER. When evaluating just the significantly upregulated genes the top common molecular processes across all three cell types are the same as shown in FIG. 6. Previously, predictive binding analysis identified three molecular processes most likely affected by this regulation of this protein, which included cellular senescence, DNA replication, and cell cycle. In the both ASCs and ACAN/Col2 ASCs groups, a top process affected by ZNF865 is ribosomal biogenesis, which may indicate that the protein processing capacity of ZNF865-edited ASCs is increased.

i. ZNF865 Upregulation Stimulates Cell Cycle Progression

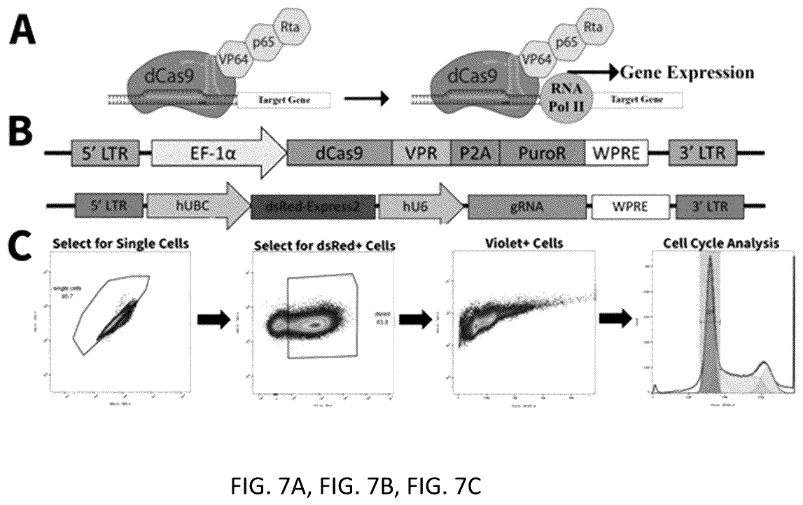

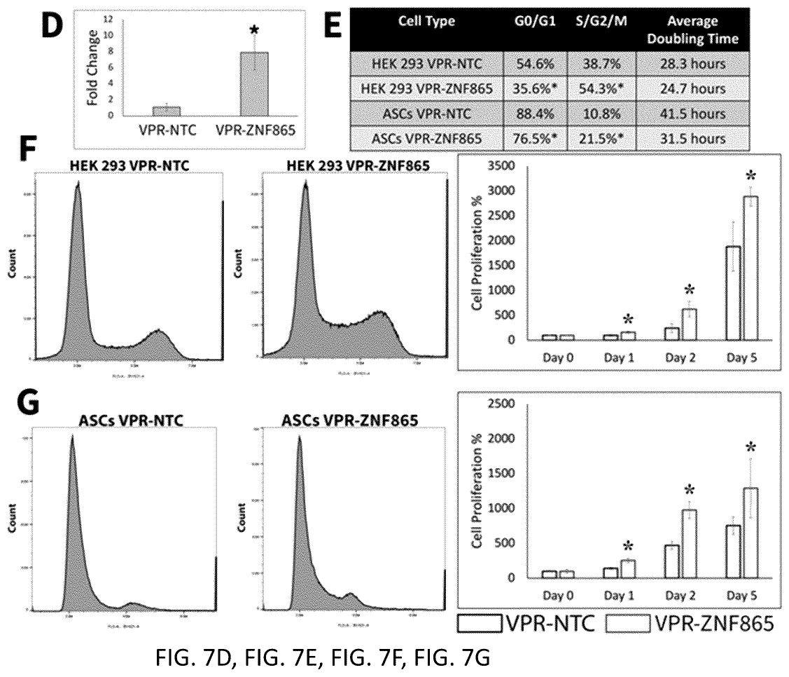

Initially changes in cell cycle due to changes in ZNF865 expression was monitored and investigated. Changes in cell cycle in HEK 293 cells and ASCs. Utilizing the CRISPRa system we can precisely target and upregulate ZNF865 (FIG. 7A, FIG. 7B) in HEK 293 and ASCs and analyze changes in cell cycle through flow cytometry (FIG. 7C) was analyzed.

CRISPRa has been shown to upregulate target gene expression precisely and robustly in mammalian cells [1,2]. qRT-PCR verified ZNF865 upregulation, with targeted upregulation of ZNF865 showing a 7.8-fold increase in ZNF865 expression compared to the -NTC baseline expression levels of ZNF865 (FIG. 7D).

Analysis of cell cycle shows differences in percentage of cells in the G0/G1 phase and the S/G2/M phase, with a significant shift of cells in the G0/G1 phase into the S/G2/M phase. HEK 293-NTC cells show 54.6% of cells in the G0/G1 phase and 38.7% in the S/G2/M phase and when ZNF865 is upregulated the percent of cells shifts to 35.6% in G0/G1 and 54.3% in the S/G2/M (FIG. 7E). ASCs show a similar shift, with the NTC cells showing 88.4% in the G0/G1 phase and 10.8% in the S/G2/M phase and those percentages shifting to 76.5% in G0/G1 and 21.5% in S/G2/M phase (FIG. 7E). In conjunction with the shift in cell cycle, an increased proliferation rates for HEK 293 cells and ASCs was observed. Both cell types show decreased doubling times decreasing from 28.3 hours in the NTC cells to 24.7 hours in ZNF865 upregulated HEK 293 cells and decreasing from 41.5 hours for the NTC and 31.5 hours in ZNF865-edited ASCs (FIG. 7E). Representative histograms displaying the shift in cell cycle and significant increases in proliferation rates for both HEK 293 (FIG. 7F) and ASCs (FIG. 7G). These results indicate that ZNF865 is a regulator of cell cycle progression and proliferation rates in both HEK 293 cells and ASCs.

ii. ZNF865 Regulates Cellular Senescence

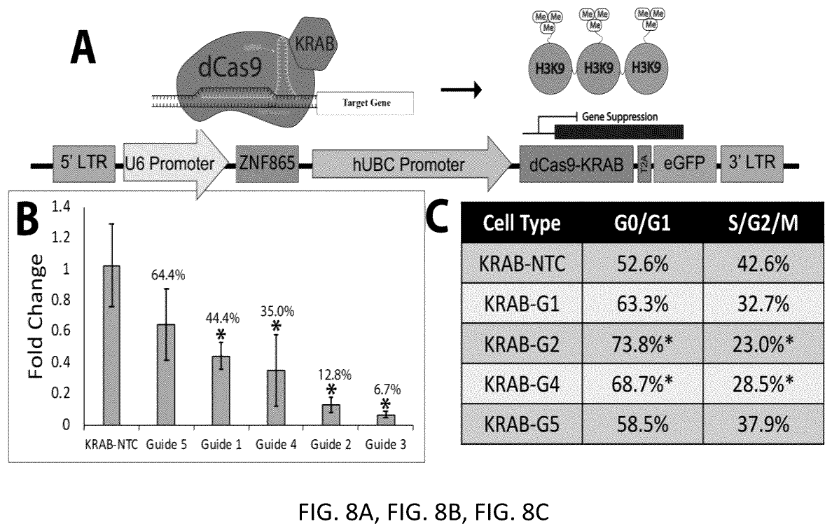

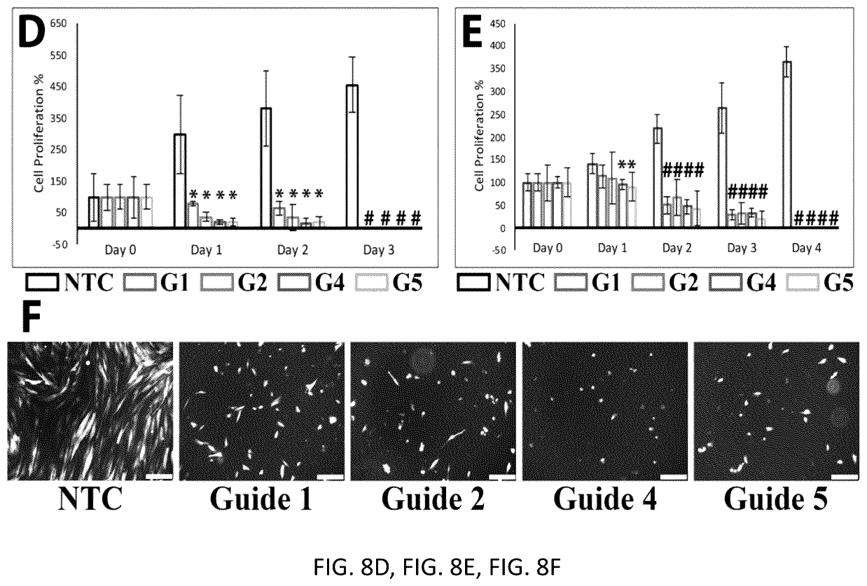

The CRISPRi system utilizes the KRAB effector molecule for targeted suppression target gene expression [3]. The KRAB molecule tri-methylates the histone, preventing transcription of the target gene, suppressing its expression (FIG. 8A) [3]. Utilizing this system and design principles ZNF865 expression to monitor cell proliferation rates and cell cycle was suppressed. qRT-PCR verified downregulation of ZNF865 expression, with 4 of the 5 guides displaying statistically significant decreases in expression and guides 2 and 3 showing near 100% suppression of ZNF865 expression (FIG. 8B). Overall, all five guides showed decreased expression of ZNF865 expression with Guide 5 dropping expression levels to 64.4%, Guide 1 to 44.4%, Guide 4 to 35.0%, Guide 2 to 12.8%, and Guide 3 to 6.7%. The data confirms the successful suppression of the target gene.

Cell cycle analysis of HEK 293 cells 96-hours after transduction shows a noticeable shift in the cell cycle. Compared to upregulation where we observed increased rates of cell cycle progression, when ZNF865 is downregulated, a build-up of cells within the G0/G1 phase compared to the NTC was observed. Guides 2 and 4 show a significant shift in the cell cycle with nearly 74% of the cells in Guide 2 being within the G0/G1 phase (FIG. 8C). This trend continues for the other guides, with a noticeable shift in cell cycle but not being significant. Furthermore, when cell proliferation rates after successful transduction with the CRISPRi system was analyzes cell death in HEK 293 cells (FIG. 8D) and ASCs (FIG. 8E) within 3 or 4 days after expression of the CRISPRi system was observed. Representative images display ASC morphology and overall cell numbers 96-hours post-transduction, with all guides showing varying degrees death (FIG. 8F). Following the successful knockdown and observed cell death in immortalized cells, the suppression of ZNF865 in primary cells using two guides, Guide 2 and Guide 3 was evaluated for a potential role in cellular senescence.

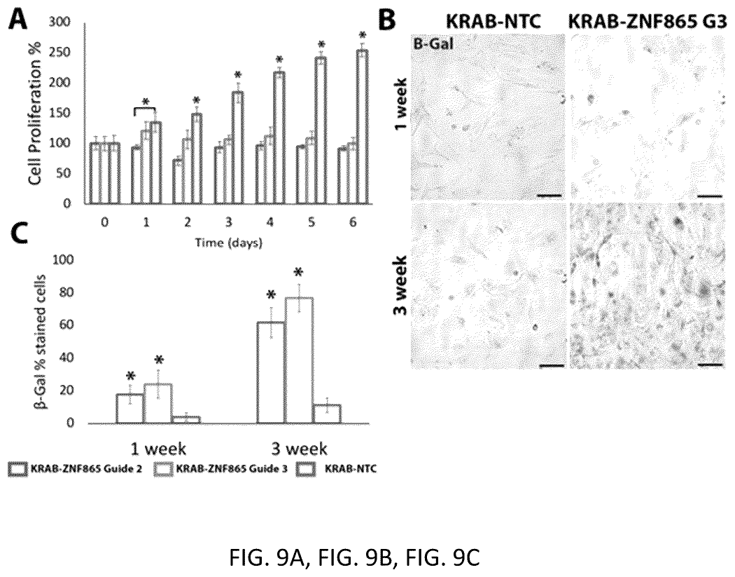

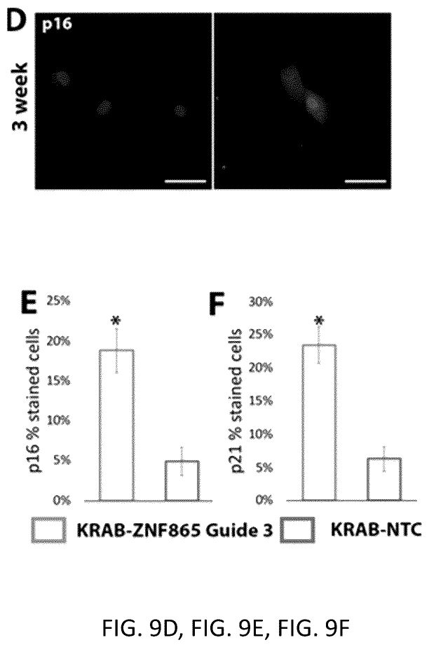

Healthy primary hNPCs were isolated from discarded surgical tissue and transduced with the ZNF865 downregulation expression cassette. Following successful transduction, hNPCs were monitored for cell proliferation over the course of 6-days and showed no proliferation over 6-days compared to the NTC hNPCs (FIG. 9A). After 1 and 3-weeks of culture, hNPCs were stained for key markers of cellular senescence, SA-β-galactosidase (β-gal) and p16 [14,15]. Quantified β-gal staining shows 17.7% and 24.0% positively stained cells after 1-week and 61.8% and 76.8% positively stained cells after 3-weeks of culture for Guide 2 and Guide 3, respectively (FIG. 9B). Representative images show the qualitative β-gal staining at 1 and 3-weeks of culture with noticeable increases in positive staining for the Guide 3 ZNF865-edited hNPCs (FIG. 9C). Furthermore, p16, a key regulator of cell cycle and a commonly stained marker indicating cellular senescence, was used to further confirm ZNF865's regulation of cellular senescence [14-16]. Representative images of p16 immunohistochemistry for Guide 3 shows increased staining at 1 and 3-weeks of culture compared to the NTC, providing additional validation for ZNF865 as a regulator of cellular senescence (FIG. 9D). Successful investigation of ZNF865 using the CRISPRi system allowed confirmation of ZNF865's role as a regulator of cellular senescence by monitoring cell proliferation rates and evaluating the presence of key cellular senescence markers in hNPCs when ZNF865 expression is suppressed.

iii. ZNF865 Upregulation Amplifies Cell Phenotype

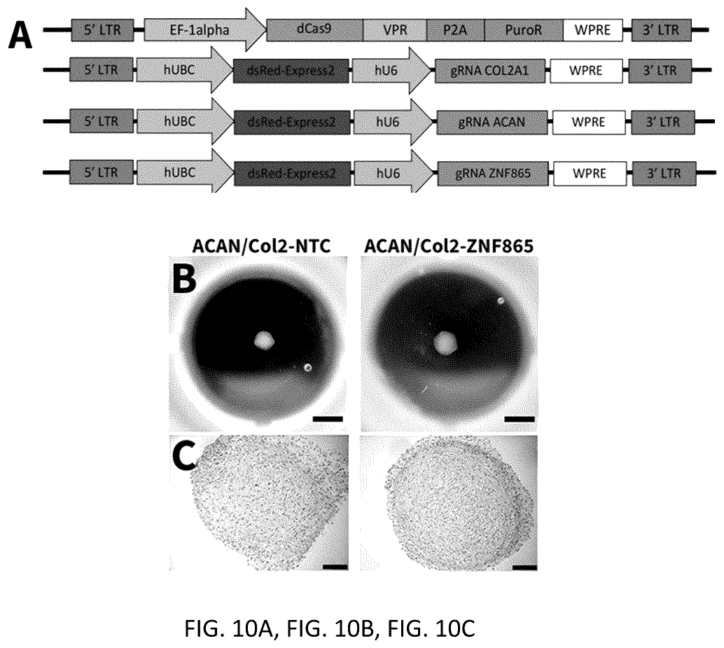

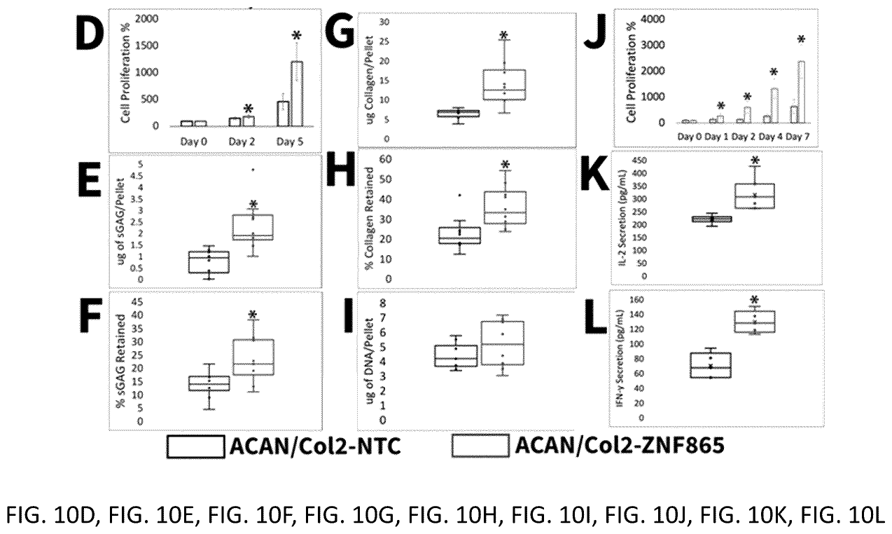

Multiplex CRISPRa upregulation showed the ability to drive a chondrogenic phenotype in ASCs, without the use of growth factors, by upregulating the genes aggrecan (ACAN) and Col2A1 (Col2) [2]. 3D pellet culture was used to evaluate protein processing and cartilaginous ECM deposition in vitro for ZNF865-edited ACAN/Col2 upregulated ASCs. ZNF865 was multiplex upregulated in conjunction with ACAN and Col2 using the dCas9-VPR CRISPRa system (FIG. 10A). After 21-days of pellet culture ZNF865-edited pellets qualitatively showed increases in overall tissue deposition (FIG. 10B) and sGAG deposition (FIG. 10C) compared to ACAN/Col2-NTC edited cells. In monolayer, ACAN/Col2-ZNF865 ASCs proliferate significantly faster compared to ACAN/Col2-NTC edited cells (FIG. 10D). Biochemical analysis shows ZNF865-edited cells produce significantly more g of sGAG/pellet with 2.28 μg/pellet compared to 0.97 μg/pellet (FIG. 10E) and retention of sGAGs within the pellet showing 22.6% in ZNF865-edited compared to 13.7% in NTC-edited (FIG. 10F). Collagen content shows significant increases in collagen per pellet with 13.84 μg/pellet compared to 6.41 μg/pellet (FIG. 10G), and retention of collagen compared to the NTC with 35.8% retained in ZNF865-edited and only 22.3% in NTC-edited (FIG. 10H). Additionally, there is no significant difference in jg of DNA/pellet with 5.16 μg/pellet in ZNF865-edited ASCs compared to 4.36 μg/pellet in NTC-edited ASCs, and there is twice as much sGAG and collagen produced in ZNF865-edited pellets compared to the NTC, indicating that ZNF865 dramatically increased the protein processing rates of individual cells in 3D culture (FIG. 10I). Furthermore, ZNF865-edited ASCs still exit the cell cycle and undergo chondrogenesis as expected in pellet culture. The results provide evidence the phenotype and increasing the rate of protein processing within the cell while not modifying the overall cell phenotype is amplified.

To examine ZNF865's affect in a nonadherent cell type, ZNF865 upregulation was performed in Jurkat cells. Similar trends in Jurkat cells were observed in the ACAN/Col2-edited ASCs, where ZNF865 upregulation significantly increased proliferation rates in Jurkat cells compared to the NTC (FIG. 10J). Significant increases in IL-2 secretion with averages of 314.7 pg/mL in ZNF865-edited cells compared to 219.7 pg/mL in NTC-edited cells (FIG. 10K) and significant increases in IFN-γ secretion, with averages of 129.2 pg/mL in ZNF865-edited cells compared to 69.9 pg/mL in NTC-edited cells (FIG. 10L) were observed. The results further confirm and validate the findings that upregulation of ZNF865 increasing the rates of protein processing within the cell and amplifies overall cell phenotype.

iv. Multiplex ACAN/Col2-ZNF865 Upregulation Amplifies Cartilage Deposition in Engineered Disc

Previously, DAPS have been shown to be a viable treatment option for treating disc degeneration. DAPS is a cell seeded total artificial intervertebral disc replacement with the potential to restore structure and function compared to current standard treatment techniques [17,18]. Currently, DAPS maturation occurs utilizing growth factors to produce appropriate matrix deposition [17-20], however the ACAN/Col2-ZNF865 ASCs produce comparable functional matrix without the use of growth factors.

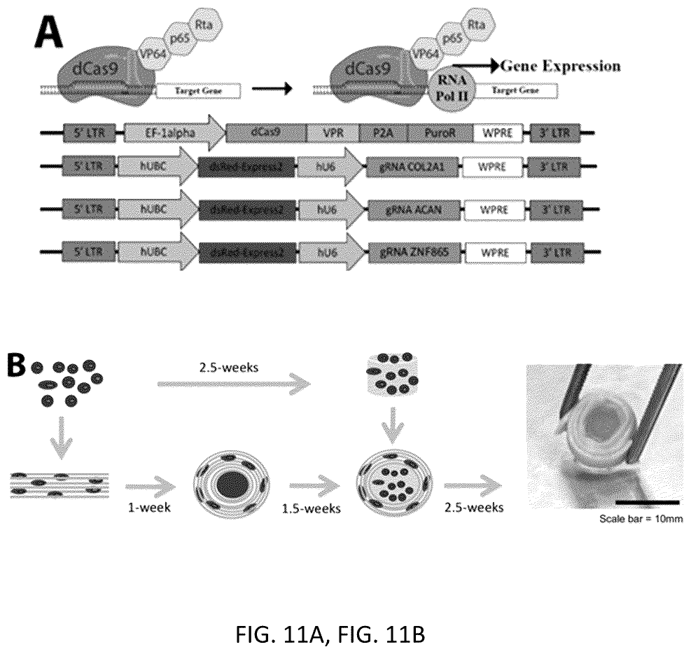

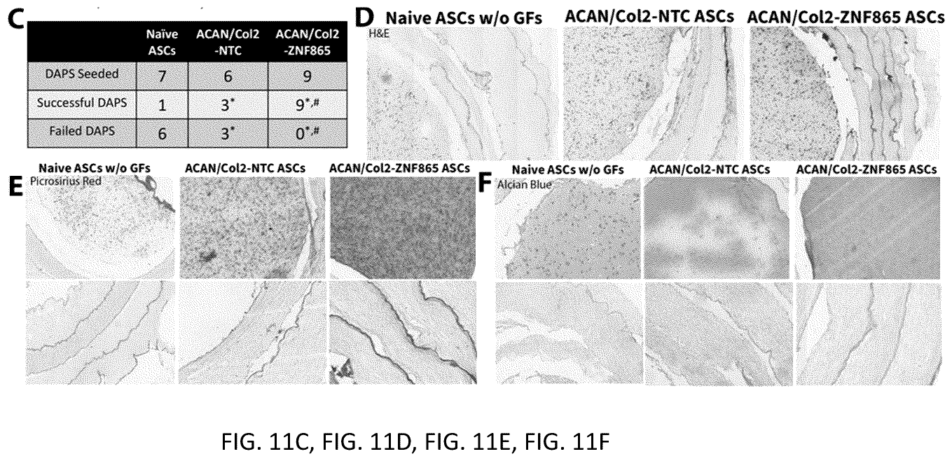

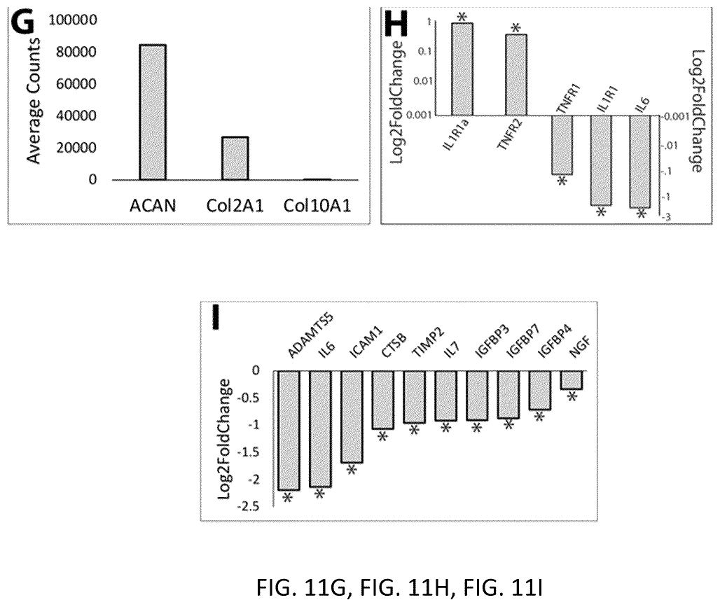

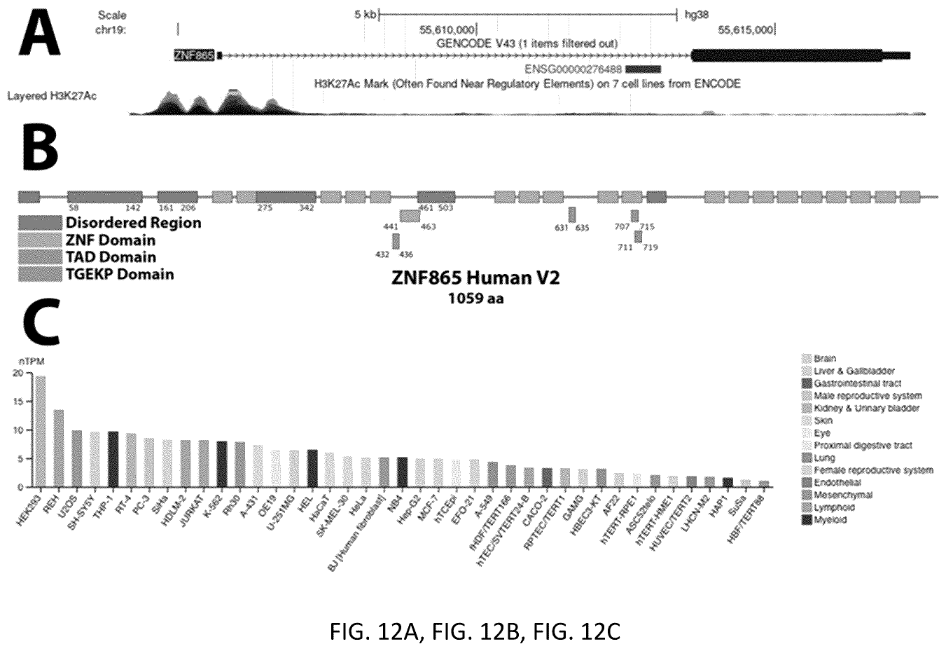

To evaluate ACAN/Col2-ZNF865 function in a tissue engineering application, DAPS were seeded with naïve ASCs, ACAN/Col2 ASCs, and ACAN/Col2-ZNF865 ASCs to investigate cartilage deposition. Naïve ASCs, multiplex CRISPRa ACAN/Col2, or multiplex ACAN/Col2-ZNF865 ASCs (FIG. 11A) were seeded onto AF and NP regions of DAPS and cultured for a total of 5-weeks (FIG. 11B). After 5-weeks of culture, the naïve ASCs were able to seed 7 total DAPS but only 1 successful DAPS was considered successful after 5-weeks of culture, ACAN/Col2 ASCs were able to seed 6 DAPS with 3 successful DAPS, however our ACAN/Col2-ZNF865 cells were able to seed 9 total DAPS and all 9 were deemed successful (FIG. 11C). The data confirms previous observations showing increased proliferation rates for ZNF865-edited DAPS compared to both control groups and the effectiveness of ZNF865-edited DAPS to produce ECM within DAPS. Alcian blue and picrosirius red combinatorial stain displays tissue deposition for all three cell types with increased staining in both the ACAN/Col2 ASCs and ACAN/Col2-ZNF865 ASCs compared to the naïve ASCs and dramatic increased staining for ZNF865-edited ASCs (FIG. 11D). Individual stains further confirm our results with picrosirius red staining showing increases in collagen deposition in ACAN/Col2 and ACAN/Col2-ZNF865 DAPS compared to the naïve control (FIG. 11E). Alcian blue staining shows the same trend, with more staining for ACAN/Col2 and dramatically darker staining for ACAN/Col2-ZNF865 DAPS compared to the naïve control (FIG. 11F). The results show the effectiveness of multiplex upregulation of CRISPRa to drive a chondrogenic phenotype without the use of growth factors.

1. A CRISPR-Cas system comprising one or more vectors comprising:
a) one or more nucleotide sequences encoding a CRISPR-Cas system guide RNA (gRNA), wherein the gRNA hybridizes with a target sequence of ZNF865 in a cell; and
b) a nucleotide sequence encoding a deactivated Cas protein fused to a transcriptional activator,
wherein components a) and b) are located on the same or different vectors of the same system, wherein the gRNA targets and hybridizes with the target sequence and directs the deactivated Cas protein fused to a transcriptional activator to the ZNF865.

2. The CRISPR-Cas system of claim 1, wherein the gRNA sequence is any of the gRNA sequences in Table 1.

3. The CRISPR-Cas system of claim 1, wherein
the target sequence is a nucleic acid sequence upstream of a transcriptional start site of ZNF865.

4. The CRISPR-Cas system of claim 1, wherein
the transcriptional activator is VPR, VP64, SAM, CNP, SPH, SunTag, or p300.

5. The CRISPR-Cas system of claim 1, wherein
a promoter is operably linked to the one or more nucleotide sequences encoding a CRISPR-Cas system gRNA.

6. The CRISPR-Cas system of claim 1, wherein
a regulatory element is operably linked to the nucleotide sequence encoding a deactivated Cas protein fused to a transcriptional activator.

7. The CRISPR-Cas system of claim 1, wherein
the cell is a eukaryotic cell.

8. The CRISPR-Cas system of claim 7, wherein the eukaryotic cell is a mammalian cell.

9. The CRISPR-Cas system of claim 8, wherein the mammalian cell is a human cell.

10. The CRISPR-Cas system of claim 9, wherein the human cell is a stem cell or T cell.

11. The CRISPR-Cas system of claim 1,
wherein the system is packaged into a single lentiviral, adenoviral or adeno-associated virus particle.

12. A method of increasing ZNF865 in a cell comprising contacting a cell with
a) a vector comprising a nucleic acid sequence encoding a ZNF865 protein;
b) a recombinant ZNF865 protein; or
c) one or more vectors comprising one or more nucleic acid sequences encoding a ZNF865-specific guide RNA (gRNA) and a dCas9 fused to a transcriptional activator, wherein the transcriptional activator upregulates ZNF865.

13. The method of claim 12, wherein contacting a cell comprises administering to
a subject comprising the cell
a) a vector comprising a nucleic acid sequence encoding a ZNF865 protein;
b) a recombinant ZNF865 protein; or
c) one or more vectors comprising one or more nucleic acid sequences encoding a ZNF865-specific guide RNA (gRNA) and a dCas9 fused to a transcriptional activator, wherein the transcriptional activator upregulates ZNF865.

14. The method of claim 12, wherein the
gRNA hybridizes with a target sequence of ZNF865 in the cell.

15. The method of claim 12, wherein the gRNA sequence is any of the gRNA sequences in Table 1.

16. The method of claim 12, wherein the target sequence is a nucleic acid sequence upstream of a transcriptional start site of ZNF865.

17. The method of claim 12, wherein the transcriptional activator is VPR, VP64, SAM, CNP, SPH, SunTag, or p300.

18.-24. (canceled)

25. A CRISPR-Cas system comprising one or more vectors comprising:
a) one or more nucleotide sequences encoding a CRISPR-Cas system guide RNA (gRNA), wherein the gRNA hybridizes with a target sequence of DNA locus in a cell; and
b) a nucleotide sequence encoding a deactivated Cas protein fused to ZNF865,
wherein components a) and b) are located on the same or different vectors of the same system, wherein the gRNA targets and hybridizes with the target sequence and directs the deactivated Cas protein fused to ZNF865 to the DNA locus.

26. The CRISPR-Cas system of claim 25, wherein the target sequence is a nucleic acid sequence upstream of a transcriptional start site of Aggrecan or Collagen II.

27. The CRISPR-Cas system of claim 25,
wherein the gRNA sequence is specific for ACAN or COL2.

28.-92. (canceled)
